(19) United States
(12) Patent Application Publication
(10) Pub. No.: US 2023/0357761 A1
(43) Pub. Date: Nov. 9, 2023

(54) ACTIVATORS OF TYPE III CAS PROTEINS

(71) Applicant: THE REGENTS OF THE UNIVERSITY OF CALIFORNIA, Oakland, CA (US)

(72) Inventors: Jennifer Doudna, Berkeley, CA (US); Patrick Hsu, Berkeley, CA (US); David Savage, Berkeley, CA (US); Tina Y. Liu, Berkeley, CA (US); Shrutee Jakhanwal, Berkeley, CA (US); Noam Prywes, Berkeley, CA (US); Gavin J. Knott, Berkeley, CA (US); Brittney Wai-Ling Thornton, Berkeley, CA (US); Dylan C.J. Smock, Berkeley, CA (US); Emeric J. Charles, Berkeley, CA (US); Shineui Kim, Berkeley, CA (US)

(21) Appl. No.: 18/245,183

(22) PCT Filed: Sep. 20, 2021
(86) PCT No.: PCT/US2021/051053

§ 371 Date: Mar. 14, 2023

Related U.S. Application Data
(60) Provisional application No. 63/080,253, filed on Sep. 18, 2020.

(51) Int. Cl. C12N 15/11; C12N 9/22; C12Q 1/6816; C12Q 1/34

(52) U.S. Cl. CPC C12N 15/11; C12N 9/22; C12Q 1/6816; C12Q 1/34; C12N 2310/3341; C12N 2310/18; G01N 2333/922

(57) ABSTRACT

Described herein are compositions and systems comprising activators of type III accessory nucleases and methods of using these compositions and systems.

### CROSS-REFERENCE TO RELATED APPLICATIONS

This application claims the benefit of U.S. Patent Application Ser. No. 63/080,253, filed on Sep. 18, 2020. The disclosure of the prior application is considered part of (and is incorporated by reference in) the disclosure of this application.

### TECHNICAL FIELD

The present invention concerns methods and compositions for activation of Type III Cas RNA-cleaving proteins (such as Csm6, Csx1) and other cyclic oligoadenylate (cA)-activated nucleases (such as Can1 or NucC), methods of using these activators in nucleic acid detection systems for rapid and sensitive detection of any target nucleic acid sequence.

### BACKGROUND

Clustered regularly interspaced short palindromic repeats (CRISPR) were discovered in the late 1980s. While the notion that these sequences are involved in bacterial defense systems was suggested over the subsequent decades, it was not until the mid to late 2000s that it became more widely accepted. During that time several papers elucidated the basics of this acquired immunity system: foreign DNA sequences (e.g., from plasmids and viruses) flanked by palindromic repeats are incorporated in into the host genome, and their RNA products direct Cas complexes to cut nucleic acids containing complementary sequences.

Simplified complexes of CRISPR-associated (Cas) proteins in combination with engineered guide RNAs were later shown to be able to locate and cleave specific DNA sequences. This led to an explosion of novel technologies, especially genome editing. Further research has shown that these proteins may be used to edit genomes in vivo. CRISPR systems are found in archaea and many bacteria. In addition to their more widely recognized ability to target DNA, some types of Cas proteins also have activity that targets RNA. For example, the Cas13 family of enzymes, such as Cas13a, Cas13b, Cas13c, and Cas13d, have two RNA endonuclease (RNase) domains.

The non-specific ribonuclease (RNase) or deoxyribonuclease (DNase) activity of some CRISPR-associated proteins may be dormant until activated by the binding of other factors to the protein or protein complex. As such, Cas13, Cas12 or Cas14 enzymes can be programmed with a guide RNA that recognizes a desired target sequence, wherein target recognition also activates a non-specific RNase or DNase activity. This can be used to release a detectable label, such as a quenched fluorescent reporter, leading to a detectable signal such as fluorescence. For example, robust RNA-stimulated cleavage of trans substrates shown by the Cas enzyme Cas13a (also known as C2c2) has been employed as a means of detecting specific RNAs within a pool of transcripts (see East-Seletsky et al. (2016) Nature 538(7624): 270-273; U.S. Pat. No. 10,337,051). Other examples include the SHERLOCK (Specific High-sensitivity Enzymatic Report UnLOCKing) system that uses Cas13 proteins for detection of RNA targets and the DETECTR system uses Cas12 proteins for DNA targets to cleave quenched reporter molecules only in the presence of a specified RNA or DNA target sequence. See, e.g., Li et al. (2019) Trends in Biotech. 37(7):730-743; Petri & Pattanayak (2018) The CRISPR Journal 1(3):209-211; Gootenberg et al. (2017) Science 356(6336):438-442; East-Seletsky et al. (2016) Nature 538(7624):270-273; Chen et al. (2018) Science 360(6387):436-439; U.S. Patent Publication Nos. 2018/0274017 and 2019/0241954 and U.S. Pat. Nos. 10,337,051 and 10,494,664.

Among the six defined types of CRISPR-Cas systems, Type III systems exhibit a dual DNA/RNA interference activity. See, e.g., Liu et al. (2018) Curr Issues Mol Biol 26:1-14. For instance, Csm6 is part of a family of single-stranded ribonucleic acid (ssRNA) endonucleases associated with Type III CRISPR-Cas systems. The RNA cleavage activity of Csm6 can be allosterically activated by binding of either cyclic oligoadenylates (cAₙ) or short linear oligoadenylates bearing a terminal 2′-3′ cyclic phosphate (Aₙ>P). Csm6 has been used in the SHERLOCK system to amplify the detection of viral RNAs. See, e.g., U.S. Patent Publication No. 2020/0254443. However, Csm6 activation by these oligoadenylates is time limited by self-inactivation mechanisms and these sequences do not produce optimal activation of Csm6 enzymes which recognize A₄, like TtCsm6. See, e.g., Garcia-Doval et al. (2020) Nature Commun 11:1596; Gootenberg et al. (2017) Science 356(6336):438-442.

Thus, there remains a need for compositions and methods that provide sustained activation of Type III Cas proteins such as Csm6, including using these activators in the context of nucleic acid detection assays.

### SUMMARY

Disclosed herein are compositions and methods for sustained activation of Type III Cas enzymes such as Csm6 or Csx1, to achieve sustained high-level activity (kinetics) of the enzyme by reducing or eliminating self-inactivation. Such activators that activate Csm6 or Csx1 may be referred to as Type III accessory nuclease activators where Type VI nuclease activators are those which activate an associated Cas protein such as Cas13 or Cas12. The compositions include oligoadenylates with one or more modified bases and/or caging structures. These modified RNA Type III accessory nuclease activators provide sustained activation of the enzyme and are useful in any Cas-based detection method.

In one aspect, described herein is a nucleic acid sequence (e.g., comprising RNA and/or DNA) that activates a Type III enzyme (e.g., Csm6, Csx1, etc.), which in turn cleaves a reporter. The Type III accessory nuclease activator sequences described herein activate a Type III Cas enzyme (e.g., Csm6) into a non-specific nuclease but are not degraded by the activated enzyme. In addition, these Type III accessory nuclease activator sequences may activate the Type III Cas enzyme to exhibit strong enzyme activity. In certain embodiments, the Type III accessory nuclease activator activates a Csm6 protein, for example T. thermophilus (TtCsm6) protein.

In certain embodiments, the Type III accessory nuclease activator sequence comprises a modified cyclic and/or linear oligoadenylate in which one or more nucleotides are modified (e.g., replaced with synthetic bases or derivatized with chemical moieties). In one embodiment, the Type III accessory nuclease activator sequence comprises a linear A4 or A6 oligoadenylate in which one or more nucleotides are modified (e.g., replaced with synthetic residues or derivatized with chemical moieties). The one or more nucleotides may be substituted with bases including modifications such as fluorine modified bases (e.g., 2′-fluorine (fA)), methylated bases (e.g., 2′ methylations (2′OMe)), and/or bases modified with deoxy (2′deoxy) molecules or other similar modifications. In some embodiments, the Type III accessory nuclease activator sequence comprises one modified adenylate, two modified adenylates, three modified adenylates or more. In some embodiments, the modified adenylate(s) may be in any location within the linear A4 or A6 oligoadenylates. In some embodiments, the Type III accessory nuclease activator sequence comprises a single type of modification (e.g., 2′-OMe, 2′-deoxy or 2′-fluoro) while in some embodiments, the Type III accessory nuclease activator sequences comprise two types of modifications or three types of modifications where the modifications can occur in any combination at any of the adenylate bases.

In certain embodiments, the Type III accessory nuclease activator comprises a sequence in which a single replacement is made at the 2′-hydroxyl of the ribose in the second A in the linear A4 or in the third A in the linear A6, optionally with a fluorine molecule such as A-fA-AA>P or AA-fA-AAA>P. In some embodiments, the Type III accessory nuclease activator comprises one or more molecules as shown in Table 3.

In certain embodiments, the Type III accessory nuclease activator sequence further comprises additional sequences, including for example, a sequence recognized by a different enzyme (e.g., a linear chain of 1-10 or more U residues recognized by Cas13), and/or a linear chain of one or more different residues (e.g., a linear chain of 1-10 or more Cs). In certain embodiments, the activator sequence comprises a caged polyC or poly U RNA reporter, for example wherein 1, 2, 3, 4, 5, 6, 7, 8, 9, 10 or more Cs or Us, optionally further comprising one or more detectable labels (e.g., fluorescent label such as a fluorescein) and/or quenchers.

In certain embodiments, the modified Type III accessory nuclease activator further comprises additional modifications, for example modifications to other nucleotides (e.g., C, A) such as 2′-deoxy modifications 3′ to the first U to restrict the cleavage of Cas13a to the precise site that is required to release the single 2′-fluoro modified An>P (e.g., A-fA-AAUCCCCCC . . . (SEQ ID NO:42)). These modifications may improve the sensitivity and/or specificity of the detection. Improvements in detection may arise from stronger activator affinity for the Type III accessory nuclease, a resistance to cleavage or other modification, modified substrate flexibility or through other interactions that are affected by the altered composition.

In another aspect, provided herein are nucleic acid detection systems comprising one or more activators as described herein. In certain embodiments, the nucleic acid detection system comprises a Cas-based nucleic acid detection system comprising: a Cas effector protein (e.g., Cas13) that binds to a target sequence in a sample (e.g., the Type VI nuclease activator); one or more Type III accessory nuclease activators as described herein and the protein(s) (e.g., Csm6) activated by these Type III accessory nuclease activators; and at least one reporter that produces a detectable signal upon cleavage by the activated Cas effector protein and/or activated Type III protein. The activated Cas effector protein and activated Type III protein cleave the same or different reporters. Optionally, one or more of the same or different activators are used in the systems described herein. In certain embodiments, the Cas13 protein is a Cas13a protein, optionally a LbuCas13 protein. Based on the cleavage preferences of the enzymes, different combinations of Cas proteins and cyclic oligoadenylate (cA)-activated nucleases could be activated by distinct sequences simultaneously, thus enabling multiplexing. In certain embodiments, the Cas protein is a Type VI Cas13 protein including, e.g., Cas13a, Cas13b, Cas13c, and/or Cas13d. In certain embodiments, the Cas protein is a Type V Cas12 or Cas14 protein, including e.g., Cas12a-Cas12e and/or Cas14a-d. In some embodiments, more than one type of Cas protein is used wherein for example, each Type V Cas protein is activated by a specific Type V nuclease activator and each Type VI Cas protein is activated by a specific Type VI nuclease activator. Any combination of Cas protein types may be used in multiplex to recognize different target nucleic acids. In some embodiments, more than one nuclease activator is used to activate a single type of Cas enzyme wherein different nuclease activators recognize different target nucleic acids.

Also described are methods of detecting a nucleic acid in a sample, the method comprising one or more of the nucleic acid detection systems, comprising the one or more activators as described herein. In certain embodiments, the methods further comprise quantifying the levels of the detectable label. The contacting step may be carried out any length of time, including seconds, minutes or hours or more (or any time therebetween), optionally seconds to 2 hours (or any time therebetween). In some embodiments, the methods described herein result in faster signal detection and/or greater specificity of signal detection. In some embodiments, the methods described herein result in signal detection at a lower activator concentration than previously achieved. In some embodiments, the methods described herein result in a longer period of signal detectability and/or a decrease in activator viability.

Also described are kits comprising one or more of the Type III accessory nuclease activators described herein, optionally further comprising one or more Type III nucleases, one or more non-Type III proteins (e.g., one or more Cas13, Cas12 and/or Cas14 proteins), one or more non-Type III protein activators, one or more additional reagents and/or instructions for using these components, for example in a nucleic acid detection system.

Accordingly, the methods and compositions of the invention comprise at least the following numbered embodiments.

### Embodiments

Accordingly, embodiments of the present subject matter described herein may be beneficial alone or in combinations, with one or more other aspects or embodiments. Without limiting the present description, certain non-limiting embodiments of the disclosure, numbered consecutively, are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below:
1. An accessory nuclease activator of a Type III Cas protein, wherein activation of the Type III Cas protein as a non-specific nuclease is sustained at high levels and is not self-limited.
2. The activator of 1, wherein the Type III Cas protein is Csm6 or Csx1, optionally a T. thermophilus (TtCsm6) protein.
3. The activator of any of the preceding, comprising one or more cyclic and/or linear oligoadenylates.
4. The activator of 3, wherein the one or more cyclic and/or linear olignodenylates comprise one or more modified bases and/or caging structures, optionally wherein the modification comprises substituting one or more bases with a non-naturally occurring base, such as a fluorinated base.
5. The activator of any of the preceding, wherein the activator comprises a linear A4 or A6 oligoadenylate.
6. The activator of 4 or 5, wherein the one or more modified bases comprise fluorinated, methylated and/or deoxy modified bases.
7. The activator of 5 or 6, wherein the substitution is at position 2 (the second A) of the A4 oligoadenylate or position 3 (the third A) of the A6 oligoadenylate, optionally with a fluorine molecule to form A-fA-AA>P or AA-fA-AAA>P.
8. The activator of any of the preceding, comprising a molecule as shown in Table 3.
9. The activator of any of the preceding, further comprising additional sequences.
10. The activator of any of the preceding comprising a sequence recognized by a different enzyme than the Type III Cas protein, optionally a Type VI Cas protein.
11. The activator of 10, wherein the sequence comprises a linear polyU chain of 1-10 U residues recognized by a Cas13 enzyme.
12. The activator of any of the preceding, further comprising a polyC sequence.
13. The activator of any of the preceding, further comprising one or more detectable labels, optionally a fluorescent label such as a fluorescein and/or one or more quenchers.
14. The activator of any of 9 to 13, wherein one or more of the polyU and/or or polyC sequences comprise one or more modified bases, optionally 2′-deoxy modifications 3′ to the first U.
15. A nucleic acid detection system comprising one or more activators of any of the preceding and the Type III Cas protein activated into a non-specific nuclease by the one or more Type III accessory nuclease activators, optionally further comprising one or more reporters that produces a detectable signal upon cleavage by the activated Type III Cas protein.
16. The nucleic acid detection system of 15, further comprising a Cas-based nucleic acid detection system comprising:
a Cas effector protein that is activated into a non-specific nuclease upon binding to a target sequence in a sample; and
and at least one reporter that produces a detectable signal upon cleavage by the activated Cas effector protein.
17. The nucleic acid detection system of 15 or 16, the Cas effector protein is Cas13 protein such as Cas13a protein, optionally a LbuCas13 protein.
18. The nucleic acid detection system of any of 15 to 17, wherein the activated Cas effector protein and activated Type III Cas protein cleave the same or different reporters.
19. A method of detecting one or more nucleic acid(s) in a sample, the method comprising:
contacting the sample with one or more of the nucleic acid detection systems according to any of 15 to 18, thereby detecting the nucleic acid in the sample, optionally, wherein the methods further comprise quantifying the levels of the detected label.
20. A kit comprising one or more activators and/or nucleic acid detection systems of any of the preceding.

These and other aspects will be readily apparent to the skilled artisan in light of disclosure as a whole.

### BRIEF DESCRIPTION OF THE DRAWINGS

FIG. 1 depicts an exemplary system using the methods and compositions described herein to detect the presence of an RNA molecule in a sample. In this schematic, a ribonucleoprotein acid (RNP) complex comprising a Cas13 protein and a guide RNA is used to recognize an RNA transcript (also referred to as the “primary activator” or “Type VI nuclease activator”). Recognition of the primary activator RNA transcript results in the activation of the Cas13 trans cleavage activity. Trans cleavage of the Type III accessory nuclease activator (SEQ ID NO:1) results in the release of a fragment (A₆>P) that is able to activate the Csm6 enzyme such that it may cleave an RNA reporter molecule. The RNA reporter comprises a fluorescent moiety that is quenched by a quenching moiety, but upon cleavage by the Csm6, the fluorescent molecule is released from quenching and is able to emit a fluorescent signal. This signal is then detected by any standard method in the art.

FIG. 2 shows a graph depicting the fluorescent signal that was generated over time using different secondary activator modifications. A Type III accessory nuclease activator comprising no base modifications (NCR142) was compared to Type III accessory nuclease activator molecules comprising 2′-OMe modifications (NCR372); 2′-deoxy modifications (NCR373) and 2′-fluoro modifications (NCR374). The results demonstrate that in this experiment, the 2′-fluoro and 2′-deoxy modified Type III accessory nuclease activator molecules resulted in the development of signal with slower kinetics than the unmodified Type III accessory nuclease activator but those signals were longer lived and reached higher levels than the unmodified activator, even though the unmodified Type III accessory nuclease activator was used at a concentration 4 times that of the modified activators.

FIGS. 3A through 3C are graphs depicting activity of the system using three types of modified Type III accessory nuclease activators and a range of concentrations, from 1 μM-4 μM FIG. 3A shows the activity observed using a 2′-fluoro modified Type III accessory nuclease activator fAfAfAA-U6 (SEQ ID NO:19). FIG. 3B shows the activity using a 2′-deoxy modified Type III accessory nuclease activator dAdAdAA-U6 (SEQ ID NO:13) and FIG. 3C shows the activity using a 2′-OMe modified Type III accessory nuclease activator mAmAmAA-U6 (SEQ ID NO:12).

FIGS. 4A through 411 are graphs depicting activity of the system using different 2-deoxy modified Type III accessory nuclease activators and a range of concentrations. Sequences used include AAAA-U6 (SEQ ID NO:1; FIG. 4A), dAdAdAA-U6 (SEQ ID NO:13; FIG. 4B), AdAAA-U6 (SEQ ID NO:14; FIG. 4C), dAAAA-U6 (SEQ ID NO:15; FIG. 4D), AAdAA-U6 (SEQ ID NO:16; FIG. 4E), AdAdAA-U6 (SEQ ID NO:17; FIG. 4F), and dAdAAA-U6 (SEQ ID NO:18; FIG. 4G). The modified Type III accessory nuclease activators have one to three 2′deoxy modifications and vary the location of the modification. FIG. 4A shows the activity using an unmodified Type III accessory nuclease activator at 0, 2 and 200 pM concentrations. FIG. 4B shows the activity where the modified Type III accessory nuclease activator comprises 2′deoxy modifications on the first three A nucleotides, while FIG. 4C shows the activity when the second A nucleotide comprises the modification. FIG. 4D shows the activity when the 2′deoxy modification is on the first A nucleotide while FIG. 4E shows the activity when the 2′deoxy modification is on the third A nucleotide. FIG. 4F shows the activity when the 2′deoxy modification is on the second and third As, while FIG. 4G shows the activity when the 2′deoxy modification is on the first and second As. FIG. 4H shows the activity in the absence of Type III accessory nuclease activator.

FIGS. 5A through 5C are graphs depicting the activity of the system using Type III accessory nuclease activators modified with 2′-fluoro nucleotides. FIG. 5A shows the activity of the unmodified Type III accessory nuclease activator (A4-U6; SEQ ID NO:1). FIG. 5B shows the activity using a modified Type III accessory nuclease activator comprising 2′-fluoro modifications on the first three A nucleotides (fAfAfAA-U6; SEQ ID NO:19). FIG. 5C shows the activity using a modified Type III accessory nuclease activator comprising a 2′-fluoro modification on the first A nucleotide (AfAAA-U6; SEQ ID NO:20).

FIGS. 6A through 6D are graphs depicting the activity of the system comparing the unmodified Type III accessory nuclease activator with three concentrations of the 2′-fluoro modified activator where the modification is located on the second A nucleotide. FIG. 6A shows the activity observed for the unmodified Type III accessory nuclease activator while FIG. 6B shows the activity using the modified activator at a concentration of 2 μM. FIG. 6C shows the activity using a concentration of 0.2 μM and FIG. 6D shows the activity using a concentration of 0.1 μM. Note the differences in the Y-axis scale for FIG. 6A as compared with FIGS. 6B, 6C and 6D.

FIGS. 7A through 7D are graphs of the data depicted in FIGS. 6A through 6D but showing only the first 60 minutes of each experiment. FIG. 7A shows the activity observed for the unmodified Type III accessory nuclease activator while FIG. 7B shows the activity using the modified activator at a concentration of 2 μM. FIG. 7C shows the activity using a concentration of 0.2 μM and FIG. 7D shows the activity using a concentration of 0.1 μM. Note the differences in the Y-axis scale for FIG. 7A as compared with FIGS. 7B, 7C and 7D.

FIGS. 8A through 8E depict the activity using a 2′-fluoro modified Type III accessory nuclease activator where cleavage of the Type III accessory nuclease activator by activated Cas13 trans cleavage activity is constrained to a specific location. FIG. 8A shows the two Type III accessory nuclease activators used where NCR142 (SEQ ID NO:1) is the unmodified Type III accessory nuclease activator and NCR690 (SEQ ID NO:2) is the modified Type III accessory nuclease activator. The cleavage site is indicated with the arrow. FIG. 8B is a graph showing the activity of the unmodified Type III accessory nuclease activator. FIG. 8C is a graph showing the activity of the system using 2 μM of the modified Type III accessory nuclease activator. FIG. 8D is a graph showing the activity using 0.2 μM of the modified Type III accessory nuclease activator and FIG. 8E is a graph showing the activity using 0.1 μM of the modified Type III accessory nuclease activator. Note the differences in the Y-axis scale for FIG. 8B as compared with FIGS. 8C, 8D and 8E.

FIGS. 9A through 9D are graphs of the data depicted in FIGS. 8B through 8E but showing only the first 60 minutes of each experiment. FIG. 9A is a graph showing the activity of the unmodified Type III accessory nuclease activator. FIG. 9B is a graph showing the activity of the system using 2 μM of the modified Type III accessory nuclease activator. FIG. 9C is a graph showing the activity using 0.2 μM of the modified Type III accessory nuclease activator and FIG. 9D is a graph showing the activity using 0.1 μM of the modified Type III accessory nuclease activator. Note the differences in the Y-axis scale for FIG. 9A as compared with FIGS. 9B, 9C and 9D.

FIGS. 10A and 10B are graphs depicting the activity of the Cas13 system alone (FIG. 10A) as compared to the Cas13 system combined with Csm6 activity (FIG. 10B). The data demonstrates a 100-fold increase in sensitivity when Csm6 activity is added to Cas13 detection alone.

FIGS. 11A and 11B are graphs depicting the activity of the Cas13+Csm6 system for a different Cas13 primary activator and with two multiplexed guides. The results shown in FIG. 11A (blown up in FIG. 11B) demonstrate detectable signal above controls at 2 fM of target.

### DETAILED DESCRIPTION

Type III CRISPR-Cas systems include several of families of proteins, such as Csm6 and Csx1, that are activated by cyclic oligoadenylates (cA(n)) or linear oligoadenylates with a 2′,3′-cyclic phosphate termini (A(n)>P). Cleavage of a nucleic acid sequence by an RNase to generate a linear oligoadenylate with exactly 4 or 6 A's and the 2′,3′-cyclic phosphate terminus (A4>P or A6>P) leads to activation of Csm6/Csx1 for cleavage of a fluorescent RNA reporter. The linear A4 or A6 can be incorporated into an RNA sequence (e.g., A4-U6 or A6-U5) such that activation of Csm6 only occurs upon removal of the U-containing sequence by Cas13, a programmable RNA-guided RNase that preferentially cleaves the phosphodiester bond that is 5′ to U's and generates products with 2′,3′-cyclic phosphates. Csm6 is normally inactivated through self-cleavage of its activator, leading to low sensitivity when coupled with a Cas13-based RNA detection system.

Current CRISPR-based nucleic acid detection methods involving Cas proteins in conjunction with Type III proteins (e.g., Csm6/Csx1) exploit non-specific cleavage of a reporter by activated Csm6 to amplify the signal obtained from Cas-based detection. In such methods, upon binding of the guide RNA of the Cas protein complex to the target nucleic acid (e.g., RNA), the Cas enzyme is activated. The detection assays are designed such that non-specific cleavage by the Cas enzyme (e.g., of a reporter) releases an oligoadenylate (cyclic or linear) that activates the Type III (e.g., Csm6) enzyme to non-specifically cleave the reporter, thereby amplifying the signal obtained in the detection assay. However, current methods can be limited by the fact that the Type III enzyme activity is time-constrained (by self-inactivating mechanisms).

Thus, described herein are molecules that activate Type III accessory proteins (e.g., Csm6) in a sustained manner and at high activity (at least retain kinetics). These molecules can generate an exponential signal upon detection of a target nucleic acid in a Cas-based detection system and provide efficient detection of the target sequence. The activators described herein can be used in any nucleic acid detection system to provide sensitive and rapid detection of any target DNA or RNA, including for detection of transcriptional states, cancers, or pathogens such as bacteria or viruses, including coronaviruses such as SARS-CoV-2 (associated with COVID-19 disease).

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art.

### Definitions

“Oligonucleotide,” “polynucleotide,” and “nucleic acid,” are used interchangeably herein. These terms may refer to a polymeric form of nucleic acids of any length, strandedness (double or single), and either ribonucleotides (RNA) or deoxyribonucleotides (DNA), and hybrid molecules (comprising DNA and RNA). The disclosed nucleic acids may also include naturally occurring and synthetic or non-natural nucleobases. Natural nucleobases include adenine (A), thymine (T), cytosine (C), guanine (G), and uracil (U). Synthetic or non-natural nucleobases can include bases modified with moieties such as fluorine groups, methyl groups and the like.

“Complementarity” refers to a first nucleic acid having a first sequence that allows it to “base pair,” “bind,” “anneal”, or “hybridize,” to a second nucleic acid. Binding may be affected by the amount of complementarity and certain external conditions such as ionic strength of the environment, temperature, etc. Base-pairing rules are well known in the art (A pairs with T in DNA, and with U in RNA; and G pairs with C). In some cases, RNA may include pairings where G may pair with U.

Complementarity does not, in all cases, indicate complete or 100% complementarity. For example, complementarity may be less than 100% and more than about 60%.

“Protein,” “peptide,” “polypeptide” are used interchangeably. The terms refer to a polymeric form of amino acids of any length, which may include natural and non-natural residues. The residues may also be modified prior to, or after incorporation into the polypeptide. In some embodiments, the polypeptides may be branched as well as linear.

“Programmed,” in reference to a Cas protein, refers to a Cas protein that includes a guide RNA that contains a sequence complementary to a target sequence. Typically, a programmed Cas protein includes an engineered guide RNA.

“Cas protein” is a CRISPR-associated protein. The presently disclosed Cas proteins possess a nuclease activity that may be activated upon binding of a target sequence to a guide RNA bound by the Cas protein. As disclosed in more detail below, the guide RNA may, with other sequences, comprise a crRNA, which may, in some embodiments, be processed from a pre-crRNA sequence. In an embodiment, the guide RNA sequence may include natural or synthetic nucleic acids, for example modified nucleic acids such as, without limitation, locked nucleic acids (LNA), 2′-o-methylated bases, or even ssDNA (single stranded DNA). Cas proteins may be from the Cas12 or Cas13 group, which may be derived from various sources known to those of skill in the art. Type III accessory nucleases include but are not limited to Csm6, Csx1, Can1, NucC and any other proteins that contain a cA-binding “sensor” domain and an effector nuclease domain as well as homologues, orthologues, and/or functional fragments of any Type III accessory nuclease (see Makarova et al (2014) Front Genet. 5:102).

The Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

“Coding sequences” are DNA sequences that encode polypeptide sequences or RNA sequences, for example guide RNAs. Coding sequences that encode polypeptides are first transcribed into RNA, which, in-turn, may encode the amino acid sequence of the polypeptide. Some RNA sequences, such as guide RNAs may not encode amino acid sequences.

“Native,” “naturally-occurring,” “unmodified” or “wild-type” describe, among other things, proteins, amino acids, cells, nucleobases, nucleic acids, polynucleotides, and organisms as found in nature. For example, a nucleic acid sequence that is identical to that found in nature, and that has not been modified by man is a native sequence.

By “hybridizable” or “complementary” or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (e.g., dsRNA duplex) of a guide RNA molecule; of a target nucleic acid (e.g., target DNA) base pairing with a guide RNA) is considered complementary to both a U and to C. For example, when a G/U base-pair can be made at a given nucleotide position of a protein-binding segment (e.g., dsRNA duplex) of a guide RNA molecule, the position is not considered to be noncomplementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al. (1990) J. Mol. Biol. 215, 403-410; Zhang and Madden (1997) Genome Res. 7:649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (1981) Adv. Appl. Math. 2:482-489.

“Binding” as used herein (e.g., with reference to an RNA-binding domain of a polypeptide, binding to a target nucleic acid, and the like) refers to a non-covalent interaction between macromolecules (e.g., between a protein and a nucleic acid; between a guide RNA and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

By “binding domain” is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., protein coding) and/or regulate translation of an encoded polypeptide.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various nucleic acids (e.g., vectors) of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e., an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Any given component, or combination of components can be unlabeled, or can be detectably labeled with a label moiety. In some cases, when two or more components are labeled, they can be labeled with label moieties that are distinguishable from one another.

“Label” or “labelling” refers to a component with a molecule that renders the component identifiable by one or more techniques. Non-limiting examples of labels include streptavidin and fluorescent molecules. The term “fluorescer” refers to a substance or a portion thereof which is capable of exhibiting fluorescence in the detectable range. The labels may be detected by a binding interaction with a label (e.g., biotin binding streptavidin) or through detection of a fluorescent signal using a fluorimeter. Other detectable labels include enzymatic labels such as luciferase, peroxidase or alkaline phosphatase. A “reporter gene” or “reporter sequence” refers to any sequence that produces a protein product that is easily measured, preferably although not necessarily in a routine assay. Suitable reporter genes include, but are not limited to, sequences encoding colored or fluorescent or luminescent proteins (e.g., green fluorescent protein, enhanced green fluorescent protein, red fluorescent protein). In some embodiments, enzymatic labels are inactivated by way of being split into two or more pieces that are linked by a nucleic acid linker that is targetable by CRISPR enzyme activity (e.g., trans cleavage following activation by the presence of an activator). Upon cleavage of the linker, the pieces of the enzymatic reporter would be able to assemble into an active enzyme that could act on a substrate to generate a detectable signal.

The term “sample” is used herein to mean any sample that includes RNA or DNA (e.g., in order to determine whether a target sequence is present among a population of polynucleotide sequences). The sample can be derived from any source, e.g., the sample can be a synthetic combination of purified RNAs/DNAs; the sample can be a cell lysate, an RNA/DNA-enriched cell lysate, or RNA/DNAs isolated and/or purified from a cell lysate. The sample may be an environmental sample, an agricultural sample or a food sample. The sample can be from a patient (e.g., for the purpose of diagnosis). The sample may be selected or derived from one or more of blood, sweat, plasma, serum, sputum, saliva, mucus, cells, excrement, urine, cerebrospinal fluid (CSF), breast milk, semen, vaginal fluid, tissue, etc. The sample can be from permeabilized cells. The sample can be from crosslinked cells. The sample can be in tissue sections. The sample can be from tissues prepared by crosslinking followed by delipidation and adjustment to make a uniform refractive index. Examples of tissue preparation by crosslinking followed by delipidation and adjustment to make a uniform refractive index have been described in, for example, Shah et al. (2016) Development 143:2862-2867 doi:10.1242/dev.138560.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to a “CRISPR/Cas effector protein” includes a plurality of CRISPR/Cas effector proteins (including the same or different Cas effector proteins) and reference to “the guide RNA” includes reference to one or more guide RNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. The compositions, methods, and systems for detecting the presence or absence of specific target nucleic acid sequence (e.g., RNA or DNA) in a sample allow for cost-effectively diagnosing a patient or sample having a viral, bacterial, parasitic, or fungal infection, or a condition, disease, or disorder by identification by the presence of one or more specific nucleic acid sequences. The compositions, methods and systems of the invention are also useful in genetic screening, cancer screening, mutational analysis, microRNA analysis, mRNA analysis, single nucleotide polymorphism analysis, etc.

### Cas Protein Activators

The Type III accessory Cas nucleases include but are not limited to Csm6, and Csx1, Can1, NucC and any other proteins or homologues, orthologues or functional fragments thereof that contain a CARF “sensor” domain and an effector nuclease domain (Makarova et al. (2014), ibid) activators disclosed herein include any molecule (e.g., RNA) which generates sustained activation (e.g., by limiting or preventing self-inactivating mechanisms such as degradation of the activator by the activated protein) of the Cas protein as a non-specific nuclease while maintaining the fast kinetics. The molecules (also referred to as “RNA activators” or “activation sequences” or “activators”) can be modified in any way to provide sustained, robust activation as compared to cyclic and/or linear oligoadenylates currently used. Once activated, the Cas protein cleaves RNA indiscriminately, similar to the collateral effect of Cas13 enzymes. Thus, in addition to detection effector modification of reporter constructs, the activated Type III enzyme can be used in conjunction with another CRISPR enzyme (e.g., Cas13, Cas12 or Cas14) for signal amplification. Thus, the activators can be used to increase sensitivity of the assay and decrease cost.

In any of the systems described herein, one or more of the components (e.g., activator, one or more guide molecules, amplifier sequences, and/or reporters) may be caged (e.g., by caging structures or molecules). In certain embodiments, the cage comprises or creates a molecule (e.g., oligonucleotide sequence) having a stem-loop structure. Oligonucleotide sequences included with the Type III accessory nuclease activator or Type VI nuclease activator may comprise DNA and/or RNA bases and, in addition, one or more of the DNA and/or RNA bases may be modified nucleotide bases, optionally comprising one or more locked nucleic acid (LNA) or moieties and/or 2′-OMe RNA. One or more caging structures may be used, for example wherein one or more of the amplifier sequences comprising caging structures on their 3′ and/or 5′ ends. One or more trans caging molecules may be also used in any of the nucleic acid systems described herein.

The Type III Cas protein activator sequences can comprise a cyclic or linear oligoadenylate that is modified at one or more residues. In certain embodiments, the activator sequences comprise modified linear A4 or A6 oligoadenylates in which one or more residues are replaced. Non-limiting examples of such replacements include 2′-fluorine (fA) and/or deoxy (see e.g., Kawasake et al. (1993) J Med Chem 36(7):831041) and the like.

In certain embodiments, a single replacement is made at the 2′-hydroxyl of the ribose in the second A in the linear A4 or in the third A in the linear A6. Such single 2′-fluoro modified poly A RNA oligonucleotides (A-fA-AA>P or AA-fA-AAA>P) provide surprising and unexpected benefits in terms activation of the enzyme (e.g., Csm6) in maintaining the fast kinetics of non-specific cleavage (to cleave the reporter and allow detection) while reducing or eliminating self-inhibition mechanisms such as degradation of the linear oligoadenylate by the activated enzyme.

Any of the RNA activators of Type III Cas proteins described herein (e.g., single 2′-fluoro-modified polyA activator) can further comprise any additional sequences, for example one or more caging structures, one or more sequences recognized by a different enzyme, such as a linear chain of U's, and is thus cleavable by Cas13 upon Cas13's activation by a complementary sequence of RNA. Such molecules can be used to generate sustained activation of Csm6 in the context of a Cas13 RNA detection system, thereby amplifying the signal obtained in the presence of the target of the detection system. Any number of U residues may be used, including but not limited to 1, 2, 3, 4, 5, 6, 7, 8, 9, 10, or more.

In still further embodiments, any of the activator sequences described herein (e.g., single 2′-fluoro-modified activator) is followed by a linear chain of C's (Cn). This substrate can be acted upon by a pre-activated Csm6 (e.g., by Cas13) to produce A-fA-AA>P or AA-fA-AAA>P, which initiates a sustained feed-forward loop and prevents self-degradation of the activator by Csm6. Restricting the cleavage site of this activator by addition of chemical modifications (such as 2′-deoxy) on positions other than the cleavage site leads to a precise cut by Csm6. The cleavage site that would result in liberation of the activator would be between the 3′-most A and the 5′-most C (e.g., A-fA-AA/dCdCdCdCdCdC (SEQ ID NO:43), where the slash represents the restricted site of cleavage and dC represents a 2′-deoxycytidine). Any number of C residues may be used, including but not limited to 1, 2, 3, 4, 5, 6, 7, 8, 9, 10, or more. In certain embodiments, the activator sequence comprises 5 C residues, optionally with 3′ and/or 5′ detectable labels and/or quenchers. In some embodiments, a C residue may be modified (e.g., using 5-methylcytosine; metdC). In certain embodiments, any of the modified activators described herein may further comprise additional modifications, for example modifications to other nucleotides of the sequence (e.g., C, A, U) such as 2′-deoxy modifications 3′ to the first U to restrict the cleavage of Cas13a to the precise site that is required to release the single 2′-fluoro modified An>P (e.g., A-fA-AAUCCCCCC . . . (SEQ ID NO:42)). This activator leads to increased sensitivity and kinetics in RNA detection when coupled with Cas13, for instance in a Cas13 nucleic acid detection system.

Also provided are nucleic acid detection systems comprising one or more Type III accessory nuclease protein activators as described herein. In certain embodiments, the nucleic acid detection system comprising the one or more activators is a Cas based nucleic acid detection system comprising a Cas effector protein (e.g., Cas13) that binds to a target sequence in a sample. Upon binding of the Cas effector protein to the target sequence, the protein is activated for non-specific cleavage of a reporter molecule to generate a detectable signal. Additionally, the one or more activators as described herein (and the cognate enzyme such as Csm6) present in the detection system amplify the signal from the same or different reporter when the Type III enzyme is activated.

One or more of the same or different Type III accessory nuclease activators can be used in the compositions and systems described herein. Thus, any of the activators described herein can be combined with one or more other activators (e.g., activators of non-Type III accessory nucleases such as activators of Cas12, Cas13 and/or Cas14 proteins) to generate even higher sensitivity and kinetics in RNA detection. Non-limiting examples of non-Type III nucleases are described in U.S Patent Publication Nos. 2019/0241954; 2020/0172886; 2019/0300908 and 2019/0300908; U.S. Pat. Nos. 10,544,428 and 10,337,051; and International Patent Publication Nos. WO 2020/181101; WO 2020/181102; WO 2020/041456; WO 2020/023529; WO 2019/104058 and WO 2019/089796. Cleavage of a fluorescent and colorimetric RNA reporter by the highly activated Type III accessory nuclease (e.g., Csm6) in either iteration generates a detectable signal. In addition, nucleotides with modified bases that are not recognized by the Type III accessory nuclease (e.g., Csm6) or non-Type III nucleases (e.g., Cas13) may also be used in the cleavable “tail” of the activators to avoid competition with the RNA reporter or other activators in the system (e.g., using 5′-methylcytosine base instead of cytosine base to avoid competition with a fluorescent reporter comprised of cytidines).

Thus, the activators described herein provide for elevated activation and kinetics of Type III Cas enzymes (e.g., Csm6 or Csx1) when coupled with a Cas13 RNA detection system, which allows for low-copy detection of any type of single-stranded RNA, including viral RNA genomes, viral RNA transcripts, and cellular RNA transcripts. In some embodiments, a signal in the system is generated in less than 1, 1, 2, 3, 4, 5, 6, 7, 8, 9, 10, 15, 20, 30, 40, 50, 60 minutes or any value therebetween. In some embodiments, the signal produced in the system is stable after 20, 30, 40, 50, 60, 70, 80, 90, 100, 110, 120 minutes or more or any value therebetween.

In addition, the activators described herein provide for increased sensitivity of detection of nucleic acids of interest as compared to systems lacking the novel activator molecules. In some embodiments, the system described is capable of detecting target nucleic acids at a concentration of 1 fM, 2 fM, 3 fM, 4 fM, 5 fM, 10 fM, 20 fM 200 fM, 2 pM or 20 pM. In some embodiments, the system has 10×, 100×, 1000× or more increased sensitivity as compared to systems lacking the modified activator molecules.

### Cas Proteins

The activators described herein can be used with any Type III Cas protein. The Cas proteins may be derived from any suitable source, including archaea and bacteria. In some embodiments, a native Cas protein may be derived from Paludibacter, Carnobacterium, Listeria, Herbinix, Rhodobacter, Leptotrichia, Lachnospiraceae, Eubacterium, or Clostridium. In some embodiments, the native Cas protein may be derived from Paludibacter propionicigenes, Carnobacterium gallinarum, Listeria seeligeri, Listeria newyorkensis, Herbinix hemicellulosilytica, Rhodobacter capsulatus, Leptotrichia wadei, Leptotrichia buccalis, Leptotrichia shahii, Lachnospiraceae bacterium NK4A179, Lachnospiraceae bacterium MA2020, Eubacterium rectale, Lachnospiraceae bacterium NK4A144, and Clostridium aminophilum.

In the Staphylococcus epidermis type III-A system, transcription across targets results in cleavage of the target DNA and its transcripts, mediated by independent active sites within the Cas10-Csm ribonucleoprotein effector protein complex (see, Samai et al. (2015) Cell 151:1164-1174). Type III-A CRISPR-Cas systems include Streptococcus thermophilus (GenBank KM222358), DGCC7710 (GenBank AWVZ01000003), LMD-9 (GenBank NC008532), Staphylococcus epidermidis RP62a (GenBank NC002976), Enterococcus italicus DSM15952 (GenBank AEPV01000074), Lactococcus lactis DGCC7167 (GenBank JX524189), Sulfolobus solfataricus P2 (GenBank AE006641), S. epidermidis RP62a (GenBank NC002976), Enterococcus italicus DSM15952 (GenBank AEPV01000074), Lactococcus lactis DGCC7167, T. thermophilus (TtCsm6, GI:55978335), S. epidermidis (SeCsm6, GI:488416649), S. mutans (SmCsm6, GI:24379650), S. thermophilus (StCsm6, GI:585230687), P. furiosus Csx1 (PfCsx1, GI:33359545) as well as the proteins disclosed in U.S. Publication No. 2020254443. In some embodiments, EiCsm6 (Enterococcus italicus; WP_007208953.1), LsCsm6 (Lactobacillus salivarius; WP_081509150.1) and/or TtCsm6 (Thermus thermophilus; WP_011229148.1) is(are) used.

In certain embodiments, the activator targets a Csm6 protein (including functional fragments, orthologues, homologues and the like of a Csm6 protein). Csm6 functions with the multiprotein Csm effector complex, but is not part of the complex. Csm6 proteins that may be activated using the compositions described herein may comprise at least one N-terminal CARF (CRISPR-associated Rossman fold) domain and/or at least one (e.g., 1 or 2) HEPN domain (higher eukaryotes and prokaryotes nucleotide-binding domain), for example at the C-terminal. In certain embodiments, Csm6 proteins form dimers. In certain embodiments, dimerization of the HEPN domains leads to the formation of a ribonuclease active site. In certain embodiments, the dimer interface of the CARF domains comprise an electropositive pocket. In certain embodiments, Csx1 can form higher-order oligomers, like tetramers and hexamers (see Molina et al. (2019) Nat Commun 10(1):4302).

In other embodiments, the activator sequence binds a Csx1 protein (including functional fragments, orthologues, homologues and the like of a Csx1 protein.

In other embodiments, the activator sequence may also bind and target a Can1 protein, for example functional fragments, orthologues, homologues, and the like of a Csx1 protein. See, e.g., McMahon, S. A., Zhu, W., Graham, S. et al. (2020) Nat Commun 11:500.doi.org/10.1038/s41467-019-14222-x.

The Cas protein(s) as described herein may be homologous to a native Cas protein. In some embodiments, the disclosed Cas protein is greater than 75%, 80%, 85%, 90%, 95%, 97%, 98%, or 99%, and less than about 100%, 99%, 98%, 97%, 95%, 90%, 85%, 80%, or 75% identical to a native Cas protein sequence.

The activator compositions, systems and methods can include one or more Cas13 proteins, for example a Cas13a with 4 currently characterized subtypes (Cas13a-d) that each exhibit significant sequence divergence apart from two consensus HEPN (Higher eukaryotes and prokaryotes nucleotide-binding domain) RNase motifs, R-X4-6-H. To defend against viral infection, Cas13 enzymes process precrRNA into mature crRNA guides in a HEPN-independent manner, followed by HEPN-dependent cleavage of a complementary “activator” target RNA in cis. Upon target-dependent activation, Cas13 is also able to cleave bystander RNAs in trans, reflecting a general RNase activity capable of both cis- and trans-cleavage. (See, e.g., U.S. Patent Publication No. 2020/0032324 and International Patent Publication No. WO 2017/218573, Konnermann et al. (2018) Cell April 19; 173(3):665-676; Zhang et al. (2018) Cell 175(1):212-223). The signature protein of Type VI-A CRISPR-Cas systems, Cas13a (formerly C2c2), is a dual nuclease responsible for both crRNA maturation and RNA-activated ssRNA cleavage (East-Seletsky et al. (2016) Nature 538(7624):270-273). Cas13a binds to precursor crRNA (pre-crRNA) transcripts and cleaves them within the repeat region to produce mature crRNAs. When the pre-crRNA is processed to the individual mature crRNAs, an 8-nucleotide piece of the repeat region that separates each of the spacer regions in a CRISPR array remains attached to the mature crRNA and is termed the “tag”. Binding to a ssRNA activator (target) sequence with complementarity to the crRNA activates Cas13a for trans-ssRNA cleavage, potentially triggering cell death or dormancy of the host organism. However, if the target or activator RNA comprises a sequence that is complementary to the tag sequence (known as the “anti-tag”) the complex is inhibited from being activated. This is thought to be a mechanism involved in preventing autoimmunity (Meeske & Marriffini (2018) Mol Cell 71:791). The Cas13a's trans-ssRNA activity can be exploited for use in releasing cage structures on RNAs; an activity that can be tuned by use of cage sequences that correspond to the preferences for the different Cas13a homologs.

In some embodiments, the Cas13 protein is a Cas13a polypeptide comprising an amino acid sequence having at least 75%, at least 80%, at least 85%, at least 90%, at least 95%, at least 98%, at least 99%, or 100%, amino acid sequence identity to any Cas13a amino acid sequence, for example a Cas13a sequence as shown in Table 1 and/or Example 2.

TABLE 1
Exemplary Cas13a proteins

| Cas13a | | Accession |
|---|---|---|
| abbreviation | Organism name | number |
| LshCas13a | Leptotrichia shahii | WP_018451595.1 |
| LwaCas13a | Leptotrichia wadei | WP_021746774.1 |
| LseCas13a | Listeria seeligeri | WP_012985477.1 |
| LbmCas13a | Lachnospiraceae bacterium MA2020 | WP_044921188.1 |
| LbnCas13a | Lachnospiraceae bacterium | WP_022785443.1 |
| | NK4A179 | |
| CamCas13a | [Clostridium] aminophilum DSM | WP_031473346.1 |
| | 10710 | |
| CgaCas13a | Carnobacterium gallinarum DSM | WP_034560163.1 |
| | 4847 | |
| Cga2Cas13a | Carnobacterium gallinarum DSM | WP_034563842.1 |
| | 4847 | |
| Pprcas13a | Paludibacter propionicigenes WB4 | WP_013443710.1 |
| LweCas13a | Listeria weihenstephanensis FSL | WP_036059185.1 |
| | R9-0317 | |
| LneCas13a | Listeriaceae bacterium FSL M6-0635 | WP_036091002.1 |
| | (Listeria newyorkensis) | |
| Lwa2cas13a | Leptotrichia wadei F0279 | WP_021746774.1 |
| RcsCas13a | Rhodobacter capsulatus SB 1003 | WP_013067728.1 |
| RcrCas13a | Rhodobacter capsulatus R121 | WP_023911507.1 |
| RcdCas13a | Rhodobacter capsulatus DE442 | WP_023911507.1 |
| LbuCas13a | Leptotrichia buccalis | WP_015770004.1 |
| LbaCas13a | Lachnospiraceae bacterium | WP_022785443.1 |
| | NK4A179 | |
| RcaCas13a | Rhodobacter capsulatus R121 | ETD76934.1 |
| EreCas13a | [Eubacterium] rectale | WP_055061018.1 |
| HheCas13a | Herbinix hemicellulosilytica | CRZ35554.1 |

Additional Cas13 proteins include BzoCas13b (Bergeyella zoohelcum; WP_002664492); PinCas13b (Prevotella intermedia; WP_036860899); PbuCas13b (Prevotella buccae; WP_004343973); AspCas13b (Alistipes sp. ZOR0009; WP_047447901); PsmCas13b (Prevotella sp. MA2016; WP_036929175); RanCas13b (Riemerella anatipestifer; WP_004919755); PauCas13b (Prevotella aurantiaca; WP_025000926); PsaCas13b (Prevotella saccharolytica, WP_051522484); Pin2Cas13b (Prevotella intermedia; WP_061868553); CcaCas13b (Capnocytophaga canimorsus; WP_013997271); PguCas13b (Porphyromonas gulae; WP_039434803); PspCas13b (Prevotella sp. P5-125, WP_0440652940); PgiCas13b (Porphyromonas gingivalis; WP_053444417); FbrCas13b (Flavobacterium branchiophilum; WP_014084666); and Pin3Cas13b (Prevotella intermedia; WP_050955369); FnsCas13c (Fusobacterium necrophorum subsp. funduhforme ATCC 51357contig00003; WP_005959231.1); FndCas13c (Fusobacterium necrophorum DJ-2 contig0065, whole genome shotgun sequence; WP_035906563.1); FnfCas13c (Fusobacterium necrophorum subsp. funduliforme 1_1_36S cont1.14; EHO19081.1); FpeCas13c (Fusobacterium perfoetens ATCC 29250 T364DRAFT_scaffold00009.9_C; WP_027128616.1); FulCas13c (Fusobacterium ulcerans ATCC 49185 cont2.38; WP_040490876.1); AspCas13c (Anaerosalibacter sp. ND1 genome assembly Anaerosalibacter massiliensis ND1; WP_042678931.1); Ruminococcus sp Cas13d, (GI: 1690532978); EsCas13d ([Eubacterium] siraeum DSM 15702; GI: 1486942132 or GI: 1486942131) and the Cas13d homologs disclosed in U.S. Patent Publication No. 2019/0062724. Exemplary Cas12 and/or Cas14 proteins that can be used in the compositions and methods described herein are described in U.S Patent Nos. 2019/0241954; 2020/0172886; 2019/0300908 and 2019/0300908 and U.S. Pat. Nos. 10,544,428 and 10,337,051; and International Patent Publication Nos. WO 2020/181101; WP 2020/181102; WO 2020/041456; WO 2020/023529; WO 2019/104058 and WO 2019/089796.

### Detection Moieties

The activators (and/or reporters for use in nucleic acid detection assays) can include one or more detection moieties, including but not limited to one or more detectable labels and/or one or more quenchers. These moieties may be linked to the activator and/or reporter at any position (e.g., 3′ and/or 5′ ends).

In some cases, the quencher moiety absorbs energy from the detectable label and then emits a signal (e.g., light at a different wavelength). Thus, in some cases, the quencher moiety is itself a signal moiety (e.g., a signal moiety can be 6-carboxyfluorescein (such as 6-FAM) while the quencher moiety can be 6-carboxy-tetramethylrhodamine), and in some such cases, the pair could also be a FRET pair. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. Patent Publication Nos. 2014/0378330, 2014/0349295 and 2014/0194611; and International Patent Publication Nos. WO 2001/42505 and WO 2001/86001, all if which are hereby incorporated by reference in their entirety.

Non-limiting examples of fluorescent labels include, but are not limited to: an Alexa Fluor™. dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein.

In some cases, a detectable label is a fluorescent label selected from: an Alexa Fluor™. dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, and Pacific Orange.

In some cases, a detectable label is a fluorescent label selected from: an Alexa Fluor™ dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC such as FAM), tetramethylrhodamine (TRITC), Texas Red, Oregon Green, Pacific Blue, Pacific Green, Pacific Orange, a quantum dot, and a tethered fluorescent protein.

Examples of ATTO dyes include, but are not limited to: ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740.

Examples of AlexaFluor dyes include, but are not limited to: Alexa Fluor™ 350, Alexa Fluor™ 405, Alexa Fluor™ 430, Alexa Fluor™ 488, Alexa Fluor™ 500, Alexa Fluor™ 514, Alexa Fluor™ 532, Alexa Fluor™ 546, Alexa Fluor™ 555, Alexa Fluor™ 568, Alexa Fluor™ 594, Alexa Fluor™ 610, Alexa Fluor™ 633, Alexa Fluor™ 635, Alexa Fluor™ 647, Alexa Fluor™ 660, Alexa Fluor™ 680, Alexa Fluor™ 700, Alexa Fluor™ 750, Alexa Fluor™ 790, and the like.

Examples of quencher moieties include, but are not limited to: a dark quencher, a Black Hole Quencher™ (BHQ™) (e.g., BHQ-0, BHQ-1, BHQ-2, BHQ-3), a Qx1 quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some cases, a quencher moiety is selected from: a dark quencher, a Black Hole Quencher™ (BHQ™) (e.g., BHQ-0, BHQ-1, BHQ-2, BHQ-3), a Qx1 quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

Examples of an ATTO quencher include, but are not limited to: ATTO 540Q, ATTO 580Q, and ATTO 612Q. Examples of a Black Hole Quencher™ (BHQ™) include, but are not limited to: BHQ-0 (493 nm), BHQ-1 (534 nm), BHQ-2 (579 nm) and BHQ-3 (672 nm).

For examples of some detectable labels (e.g., fluorescent dyes) and/or quencher moieties, see, e.g., Bao et al. (2009) Annu Rev Biomed Eng. 11:25-47; as well as U.S. Pat. Nos. 8,822,673 and 8,586,718; U.S. Patent Publication Nos. 2014/0378330; 2014/0349295; 2014/0194611; 2013/0323851; 2013/0224871; 2011/0223677; 2011/0190486; 2011/0172420; 2006/0179585 and 2003/0003486; and International Patent Publication No. WO 2001/42505 and WO 2001/86001, all of which are hereby incorporated by reference in their entirety.

In some cases, cleavage of a labeled detector can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector ssDNA can be detected by a color-shift. Such a shift can be expressed as a loss of an amount of signal of one color (wavelength), a gain in the amount of another color, a change in the ratio of one color to another, and the like.

In some cases, signal is detected using lateral flow chromatography. In a simple sandwich type of system, the sample is applied to a pad in the lateral flow device that acts as the first stage of the absorption process, and in some cases contains a filter, to ensure the accurate and controlled flow of the sample. The conjugate pad, which stores the conjugated labels and antibodies, will receive the sample. If the target is present, the immobilized conjugated antibodies and labels will bind to the target and continue to migrate along the test. As the sample moves along the device the binding reagents situated on the nitrocellulose membrane will bind to the target at the test line. A colored line will form and the density of the line will vary depending on the quantity of the target present. Some targets may require quantification to determine target concentration. This is where a rapid test can be combined with a reader to provide quantitative results.

### Methods

The compositions (activators) described herein find use in systems and methods of nucleic acid detection, including providing surprising and unexpected benefits in terms of signal detection in Cas-based detection assays.

Thus, methods of the invention include (a) providing a nucleic acid detection system (e.g., Cas13 system) comprising any of the activators as described herein and (b) measuring a detectable signal generated in the presence of the target sequence, thereby detecting the target sequence (RNA sample or reverse transcribed from DNA target sequence).

In some cases, the methods comprise contacting a target sensor comprising one or more Cas-effector enzymes programmed with one or more guide RNAs that recognize the desired target nucleic sequence(s) in the sample (e.g., viral DNA or RNA) such that the target sensor is activated into a non-specific nuclease (e.g., non-specific RNase when the target sensor comprises a Cas13 effector protein or non-specific DNase when the target sensor comprises a Cas12 effector protein). In certain cases, the target sensor comprises one Cas-effector protein and one guide RNA. The methods also comprise contacting the activated target sensor (non-specific nuclease) with a reporter molecule, which comprises a detectable label and one or more activator sequences as described herein, in which the detectable label is masked (quenched) and the activator sequence is caged (unavailable for hybridization) prior to cleavage by the non-specific nuclease. Upon cleavage, both the detectable label (e.g., fluorescent label) and the activator as described are released. Subsequently, the released activator activates the Type III Cas protein (e.g., Csm6) into an additional non-specific nuclease capable of cleaving the reporter molecule and releasing the detectable label. The methods also comprise measuring the detectable label and, optionally quantifying the levels. In some embodiments, signal in the system is generated in less than 1, 1, 2, 3, 4, 5, 6, 7, 8, 9, 10, 15, 20, 30, 40, 50, 60 minutes or any value therebetween. In some embodiments, the signal produced in the system is stable after 20, 30, 40, 50, 60, 70, 80, 90, 100, 110, 120 minutes or more or any value therebetween.

In addition, the activators described herein provide for increased sensitivity of detection of nucleic acids of interest as compared to systems lacking the novel activator molecules. In some embodiments, the system described is capable of detecting target nucleic acids at a concentration of 1 fM, 2 fM, 3 fM, 4 fM, 5 fM, 10 fM, 20 fM 200 fM, 2 pM or 20 pM. In some embodiments, the system has 10×, 100×, 1000× or more increased sensitivity as compared to systems lacking the modified activator molecules.

The contacting steps and measuring steps may be performed in the same or different containers and in liquid and/or solid supports. For example, the contacting may be performed in the same container and transferred for detection or, alternatively, the contacting and measuring steps may be performed in the same container. The contacting step of a subject methods can be carried out in a composition comprising divalent metal ions. The contacting step can be carried out in an acellular environment, e.g., outside of a cell. The contacting step can be carried out inside a cell. The contacting step can be carried out in a cell in vitro. The contacting step can be carried out in a cell ex vivo. The contacting step can be carried out in a cell in vivo.

The contacting step may be for any length of time, including but not limited to 2 hours or less (e.g., 1.5 hours or less, 1 hour or less, 40 minutes or less, 30 minutes or less, 20 minutes or less, 10 minutes or less, or 5 minutes or less, or 1 minute or less) prior to the measuring step. For example, in some cases the sample is contacted for 40 minutes or less prior to the measuring step. In some cases, the sample is contacted for 20 minutes or less prior to the measuring step. In some cases, the sample is contacted for 10 minutes or less prior to the measuring step. In some cases, the sample is contacted for 5 minutes or less prior to the measuring step. In some cases, the sample is contacted for 1 minute or less prior to the measuring step. In some cases, the sample is contacted for from 50 seconds to 60 seconds prior to the measuring step. In some cases, the sample is contacted for from 40 seconds to 50 seconds prior to the measuring step. In some cases, the sample is contacted for from 30 seconds to 40 seconds prior to the measuring step. In some cases, the sample is contacted for from 20 seconds to 30 seconds prior to the measuring step. In some cases, the sample is contacted for from 10 seconds to 20 seconds prior to the measuring step. In some embodiments, the sample is incubated with the Cas protein for less than about 2 hrs., 90 min., 60 min., 40 min., 30 min., 20 min., 10 min., 5 min., 4 min., 3 min., 2 min., 1 min., 55 sec., 50 sec., 40 sec., 30 sec., 20 sec., or 10 sec., and more than about 5 sec., 10 sec., 20 sec., 30 sec., 40 sec., 50 sec., 60 sec., 2 min., 3 min., 4 min., 5 min., 10 min., 20 min., 30 min., 40 min., 50 min., 60 min., or 90 min.

The method may be conducted at any temperature, including from about 20° C. (room temperature) to about 65° C. (or any temperature therebetween, depending on the thermostability of the particular enzyme orthologs used). In some embodiments, the assays (methods) are conducted at a physiological temperature, for example about 37° C. This allows the methods to be readily practiced in any location, including a doctor's office or home (for example by performing the assay using body temperature (e.g., holding the assay contained under the arm, against the skin, etc.). In some embodiments, the assays (methods) are conducted at 60-65° C.

The methods described herein can detect the target sequence (RNA or DNA) with a high degree of sensitivity. In some cases, a method of the present disclosure can be used to detect a target sequence present in a sample comprising a plurality of nucleotides (including the target sequence and a plurality of non-target sequences), where the target sequence is present at one or more copies per 10⁷ non-target sequences (e.g., one or more copies per 10⁶ non-target sequences, one or more copies per 10⁵ non-target sequences, one or more copies per 10⁴ non-target sequences, one or more copies per 10³ non-target sequences, one or more copies per 10² non-target sequences, one or more copies per 50 non-target sequences, one or more copies per 20 non-target sequences, one or more copies per 10 non-target sequences, or one or more copies per 5 non-target sequences). In some cases, a method of the present disclosure can be used to detect a target sequences present in a sample comprising a plurality of sequences (including the target sequences and a plurality of non-target sequences), where the target sequence is present at one or more copies per 10¹⁸ non-target sequences (e.g., one or more copies per 10¹⁵ non-target sequences, one or more copies per 10¹² non-target sequences, one or more copies per 10⁹ non-target sequences, one or more copies per 10⁶ non-target sequences, one or more copies per 10⁵ non-target sequences, one or more copies per 10⁴ non-target sequences, one or more copies per 10³ non-target sequences, one or more copies per 10² non-target sequences, one or more copies per 50 non-target sequences, one or more copies per 20 non-target sequences, one or more copies per 10 non-target sequences, or one or more copies per 5 non-target sequences).

In some cases, a method of the present disclosure can detect a target sequence (DNA or RNA) present in a sample, where the target sequence is present at from one copy per 10⁷ non-target sequences to one copy per 10 non-target sequences (e.g., from 1 copy per 10⁷ non-target sequences to 1 copy per 10² non-target sequences, from 1 copy per 10⁷ non-target sequences to 1 copy per 10³ non-target sequences, from 1 copy per 10⁷ non-target sequences to 1 copy per 10⁴ non-target sequences, from 1 copy per 10⁷ non-target sequences to 1 copy per 10⁵ non-target sequences, from 1 copy per 10⁷ non-target sequences to 1 copy per 10⁶ non-target sequences, from 1 copy per 10⁶ non-target sequences to 1 copy per 10 non-target sequences, from 1 copy per 10⁶ non-target sequences to 1 copy per 10² non-target sequences, from 1 copy per 10⁶ non-target sequences to 1 copy per 10³ non-target sequences, from 1 copy per 10⁶ non-target sequences to 1 copy per 10⁴ non-target sequences, from 1 copy per 10⁶ non-target sequences to 1 copy per 10⁵ non-target sequences, from 1 copy per 10⁵ non-target sequences to 1 copy per 10 non-target sequences, from 1 copy per 10⁵ non-target sequences to 1 copy per 10² non-target sequences, from 1 copy per 10⁵ non-target sequences to 1 copy per 10³ non-target sequences, or from 1 copy per 10⁵ non-target sequences to 1 copy per 10⁴ non-target sequences).

In some cases, a method of the present disclosure can detect a target sequence (RNA or DNA) present in a sample, where the target sequences is present at from one copy per 10¹⁸ non-target sequences to one copy per 10 non-target sequences (e.g., from 1 copy per 10¹⁸ non-target sequences to 1 copy per 10² non-target sequences, from 1 copy per 10¹⁵ non-target sequences to 1 copy per 10² non-target sequences, from 1 copy per 10¹² non-target sequences to 1 copy per 10² non-target sequences, from 1 copy per 10⁹ non-target sequences to 1 copy per 10² non-target sequences, from 1 copy per 10⁷ non-target sequences to 1 copy per 10² non-target sequences, from 1 copy per 10⁷ non-target sequences to 1 copy per 10³ non-target sequences, from 1 copy per 10⁷ non-target sequences to 1 copy per 10⁴ non-target sequences, from 1 copy per 10⁷ non-target sequences to 1 copy per 10⁵ non-target sequences, from 1 copy per 10⁷ non-target sequences to 1 copy per 10⁶ non-target sequences, from 1 copy per 10⁶ non-target sequences to 1 copy per 10 non-target sequences, from 1 copy per 10⁶ non-target sequences to 1 copy per 10² non-target sequences, from 1 copy per 10⁶ non-target sequences to 1 copy per 10³ non-target sequences, from 1 copy per 10⁶ non-target sequences to 1 copy per 10⁴ non-target sequences, from 1 copy per 10⁶ non-target sequences to 1 copy per 10⁵ non-target sequences, from 1 copy per 10⁵ non-target sequences to 1 copy per 10 non-target sequences, from 1 copy per 10⁵ non-target sequences to 1 copy per 10² non-target sequences, from 1 copy per 10⁵ non-target sequences to 1 copy per 10³ non-target sequences, or from 1 copy per 10⁵ non-target sequences to 1 copy per 10⁴ non-target sequences).

In some cases, a method of the present disclosure can detect a target sequence (RNA or DNA) present in a sample, where the target sequence is present at from one copy per 10⁷ non-target sequences to one copy per 100 non-target sequences (e.g., from 1 copy per 10⁷ non-target sequences to 1 copy per 10² non-target sequences, from 1 copy per 10⁷ non-target sequences to 1 copy per 10³ non-target sequences, from 1 copy per 10⁷ non-target sequences to 1 copy per 10⁴ non-target sequences, from 1 copy per 10⁷ non-target sequences to 1 copy per 10⁵ non-target sequences, from 1 copy per 10⁷ non-target sequences to 1 copy per 10⁶ non-target sequences, from 1 copy per 10⁶ non-target sequences to 1 copy per 100 non-target sequences, from 1 copy per 10⁶ non-target sequences to 1 copy per 10² non-target sequences, from 1 copy per 10⁶ non-target sequences to 1 copy per 10³ non-target sequences, from 1 copy per 10⁶ non-target sequences to 1 copy per 10⁴ non-target sequences, from 1 copy per 10⁶ non-target sequences to 1 copy per 10⁵ non-target sequences, from 1 copy per 10⁵ non-target sequences to 1 copy per 100 non-target sequences, from 1 copy per 10⁵ non-target sequences to 1 copy per 10² non-target sequences, from 1 copy per 10⁵ non-target sequences to 1 copy per 10³ non-target sequences, or from 1 copy per 10⁵ non-target sequences to 1 copy per 10⁴ non-target sequences).

In some cases, the threshold of detection, for a subject method of detecting a target sequence (RNA or DNA) in a sample, is 10 nM or less. The term “threshold of detection” is used herein to describe the minimal amount of target sequence that must be present in a sample in order for detection to occur. Thus, as an illustrative example, when a threshold of detection is 10 nM, then a signal can be detected when a target sequence is present in the sample at a concentration of 10 nM or more. In some cases, a method of the present disclosure has a threshold of detection of 5 nM or less. In some cases, a method of the present disclosure has a threshold of detection of 1 nM or less. In some cases, a method of the present disclosure has a threshold of detection of 0.5 nM or less. In some cases, a method of the present disclosure has a threshold of detection of 0.1 nM or less. In some cases, a method of the present disclosure has a threshold of detection of 0.05 nM or less. In some cases, a method of the present disclosure has a threshold of detection of 0.01 nM or less. In some cases, a method of the present disclosure has a threshold of detection of 0.005 nM or less. In some cases, a method of the present disclosure has a threshold of detection of 0.001 nM or less. In some cases, a method of the present disclosure has a threshold of detection of 0.0005 nM or less. In some cases, a method of the present disclosure has a threshold of detection of 0.0001 nM or less. In some cases, a method of the present disclosure has a threshold of detection of 0.00005 nM or less. In some cases, a method of the present disclosure has a threshold of detection of 0.00001 nM or less. In some cases, a method of the present disclosure has a threshold of detection of 10 pM or less. In some cases, a method of the present disclosure has a threshold of detection of 1 pM or less. In some cases, a method of the present disclosure has a threshold of detection of 500 fM or less. In some cases, a method of the present disclosure has a threshold of detection of 250 fM or less. In some cases, a method of the present disclosure has a threshold of detection of 100 fM or less. In some cases, a method of the present disclosure has a threshold of detection of 50 fM or less. In some cases, a method of the present disclosure has a threshold of detection of 500 aM (attomolar) or less. In some cases, a method of the present disclosure has a threshold of detection of 250 aM or less. In some cases, a method of the present disclosure has a threshold of detection of 100 aM or less. In some cases, a method of the present disclosure has a threshold of detection of 50 aM or less. In some cases, a method of the present disclosure has a threshold of detection of 10 aM or less. In some cases, a method of the present disclosure has a threshold of detection of 1 aM or less.

In some cases, the threshold of detection (for detecting the target sequence in a subject method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target sequence at which the target sequence can be detected). In some cases, a method of the present disclosure has a threshold of detection in a range of from 800 fM to 100 pM. In some cases, a method of the present disclosure has a threshold of detection in a range of from 1 pM to 10 pM. In some cases, a method of the present disclosure has a threshold of detection in a range of from 10 fM to 500 fM, e.g., from 10 fM to 50 fM, from 50 fM to 100 fM, from 100 fM to 250 fM, or from 250 fM to 500 fM.

In some cases, the minimum concentration at which a target sequence (DNA or RNA) can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target sequence can be detected in a sample is in a range of from 800 fM to 100 pM. In some cases, the minimum concentration at which a target sequence can be detected in a sample is in a range of from 1 pM to 10 pM.

In some cases, the threshold of detection (for detecting the target sequences), is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM) (where the concentration refers to the threshold concentration of target sequence at which the target sequence can be detected). In some cases, a method of the present disclosure has a threshold of detection in a range of from 1 aM to 800 aM. In some cases, a method of the present disclosure has a threshold of detection in a range of from 50 aM to 1 pM. In some cases, a method of the present disclosure has a threshold of detection in a range of from 50 aM to 500 fM.

In some cases, the minimum concentration at which a target sequence can be detected in a sample is in a range of from 1 aM to 1 nM (e.g., from 1 aM to 500 pM, from 1 aM to 200 pM, from 1 aM to 100 pM, from 1 aM to 10 pM, from 1 aM to 1 pM, from 100 aM to 1 nM, from 100 aM to 500 pM, from 100 aM to 200 pM, from 100 aM to 100 pM, from 100 aM to 10 pM, from 100 aM to 1 pM, from 250 aM to 1 nM, from 250 aM to 500 pM, from 250 aM to 200 pM, from 250 aM to 100 pM, from 250 aM to 10 pM, from 250 aM to 1 pM, from 500 aM to 1 nM, from 500 aM to 500 pM, from 500 aM to 200 pM, from 500 aM to 100 pM, from 500 aM to 10 pM, from 500 aM to 1 pM, from 750 aM to 1 nM, from 750 aM to 500 pM, from 750 aM to 200 pM, from 750 aM to 100 pM, from 750 aM to 10 pM, from 750 aM to 1 pM, from 1 fM to 1 nM, from 1 fM to 500 pM, from 1 fM to 200 pM, from 1 fM to 100 pM, from 1 fM to 10 pM, from 1 fM to 1 pM, from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a target sequence can be detected in a sample is in a range of from 1 aM to 500 pM. In some cases, the minimum concentration at which a target sequence can be detected in a sample is in a range of from 100 aM to 500 pM.

In some cases, a subject composition or method exhibits an attomolar (aM) sensitivity of detection. In some cases, a subject composition or method exhibits a femtomolar (fM) sensitivity of detection. In some cases, a subject composition or method exhibits a picomolar (pM) sensitivity of detection. In some cases, a subject composition or method exhibits a nanomolar (nM) sensitivity of detection.

The measuring can in some cases be quantitative, e.g., in the sense that the amount of signal detected can be used to determine the amount of target sequence present in the sample. The measuring can in some cases be qualitative, e.g., in the sense that the presence or absence of detectable signal can indicate the presence or absence of targeted sequence (e.g., virus, SNP, etc.). In some cases, a detectable signal will not be present (e.g., above a given threshold level) unless the targeted sequence(s) (e.g., virus, SNP, etc.) is present above a particular threshold concentration. In some cases, the threshold of detection can be titrated by modifying the amount of Cas effector protein of the system (e.g., sensor or amplifier), guide RNA, sample volume, and/or detector (if one is used). As such, for example, as would be understood by one of ordinary skill in the art, a number of controls can be used if desired in order to set up one or more reactions, each set up to detect a different threshold level of target sequence, and thus such a series of reactions could be used to determine the amount of target sequence present in a sample (e.g., one could use such a series of reactions to determine that a target sequence is present in the sample ‘at a concentration of at least X’).

In some cases, a method of the present disclosure can be used to determine the amount of a target sequence (RNA or DNA) in a sample (e.g., a sample comprising the target sequence and a plurality of non-target sequences). Determining the amount of a target sequence in a sample can comprise comparing the amount of detectable signal generated from a test sample to the amount of detectable signal generated from a reference sample. Determining the amount of a target sequence in a sample can comprise: measuring the detectable signal to generate a test measurement; measuring a detectable signal produced by a reference sample to generate a reference measurement; and comparing the test measurement to the reference measurement to determine an amount of target sequence present in the sample.

RNase inhibitors may be used in the methods as described herein. In some embodiments, the assay mixture may include one or more molecules that inhibit non-Cas13a-dependent RNase activity, but do not affect RNase activity by activated Cas13a proteins. For example, the inhibitor may inhibit mammalian, bacterial, or viral RNases, such as, without limitation, RNase A and RNase H. In some embodiments, the RNase Inhibitor may be added to the sample to help preserve a target nucleic acid sequence. In these embodiments, the method may include a step of adding one or more RNA preserving compounds to the sample, for example one or more RNase inhibitors.

Detecting the label may be achieved in various ways known in the art. For example, detection of colorimetric, fluorescent, or luminescent labels may be accomplished by measurement of absorbance or emission of light at a particular wavelength. In some embodiments the signal may be detected by visual inspection, microscope, or light detector.

### Target Sequences

The source of the target sequence in detection assays using one or more of the activators described herein can be any source, including mammals, viruses, bacteria, and fungi. In some embodiments, the target sequence is a microbial or viral sequence, for example a coronavirus sequence such as SARS-CoV2. In still other embodiments the target sequence is a mammalian genomic or transcribed sequence. In some embodiments, the source may be a human, non-human, or animal. In some embodiments, an animal source may be a domesticated or non-domestic animal, for example wild game. In some embodiments, the domesticated animal is a service or companion animal (e.g., a dog, cat, bird, fish, or reptile), or a domesticated farm animal.

For target sequences from pathogenic sources, the pathogen may have significant public health relevance, such as bacteria, fungus, or protozoan, and the target sequence may be found, without limitation, in one or more of coronavirus (e.g., severe acute respiratory syndrome-related coronavirus (SARS), Middle East respiratory syndrome-related coronavirus (MERS), COVID-19, etc.), Hepatitis C virus, Japanese Encephalitis, Dengue fever, or Zika virus. Any pathogen (e.g., virus, bacteria, etc.) can be detected.

A target sequence can be single stranded (ss) or double stranded (ds) DNA or RNA (e.g., viral RNA, mRNA, tRNA, rRNA, iRNA, miRNA, etc.). When the target sequence is single stranded, there is no preference or requirement for a PAM sequence in the target. However, when the target DNA is dsDNA, a PAM is usually present adjacent to the target sequence of the target DNA (e.g., see discussion of the PAM elsewhere herein). The source of the target DNA can be the same as the source of the sample, e.g., as described below. DNA can be reverse transcribed into RNA for detection by RNA detection (e.g., Cas13-based systems).

In some cases, the target sequence is a viral sequence (e.g., a genomic RNA of an RNA virus or DNA of a DNA virus). As such, the subject method can be used for detecting the presence of a viral sequence amongst a population of nucleic acids (e.g., in a sample).

Non-limiting examples of possible primary RNA targets include viral RNAs such as coronavirus (SARS, MERS, SARS-CoV-2), Orthomyxoviruses, Hepatitis C Virus (HCV), Ebola disease, influenza, polio measles and retrovirus including adult Human T-cell lymphotropic virus type 1 (HTLV-1) and human immunodeficiency virus (HIV).

Non-limiting examples of possible target DNAs include, but are not limited to, viral DNAs such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. In some cases, the target DNA is parasite DNA. In some cases, the target DNA is bacterial DNA, e.g., DNA of a pathogenic bacterium.

In some embodiments, the target nucleic acid is a DNA or RNA sequence associated with cancer. These can include genes that play a role in DNA methylation, histone modification, message splicing, and microRNA expression. Along with well known examples such as the so-called Philadelphia chromosome associated with chronic myeloid leukemia, in some embodiments, the target is a DNA associated with a translocation such as t(8;14)(q24;q32), t(2;8)(p12;q24), t(8;22)(q24;q11), t(8;14)(q24;q11), and t(8;12)(q24;q22), each associated with an alteration of C-Myc and associated with acute lymphocytic leukemia. Other examples include t(10;14)(q24;q32) which effects the LYT10 gene and is associated with B cell lymphoma (see Nambiar (2008) Biochim Biophys Acta 1786:139-152). Other targets include mutant genes associated with cancers such as BRCA2 (ovarian cancer), BMP2, 3, 4, 7 (endometrial cancer), CAGE (cervical cancer), HOXAM (ovarian cancer) and more (see Jeong et al. (2014) Front Oncol 4(12)).

In some cases, the methods and compositions of the invention are used to examine other disorders that display an altered transcriptional state. Examples include diabetes, metabolic syndrome (Hawkins et al. (2018) Peer J 6:e5062), Huntington syndrome and other neurological diseases (Xiang et al. (2018) Front Mol Neurosci 11:153) and cancer. In some cases, the methods and compositions are used to monitor response to a therapy administered for the treatment of a disorder characterized by an altered transcriptional state. In some cases, the methods and compositions are used to monitor altered transcriptional activity in a non-disease condition such as the onset of puberty, pregnancy or menopause.

### Samples

Any sample that includes nucleic acid (e.g., a plurality of nucleic acids) can be used in the compositions, systems and methods described herein. The term “plurality” is used herein to mean two or more. Thus, in some cases a sample includes two or more (e.g., 3 or more, 5 or more, 10 or more, 20 or more, 50 or more, 100 or more, 500 or more, 1,000 or more, or 5,000 or more) nucleic acids (e.g., RNAs or DNAs). A subject method can be used as a very sensitive way to detect a target sequence present in a sample (e.g., in a complex mixture of nucleic acids such as RNAs or DNAs). In some cases, the sample includes 5 or more RNAs or DNAs (e.g., 10 or more, 20 or more, 50 or more, 100 or more, 500 or more, 1,000 or more, or 5,000 or more RNAs or DNAs) that differ from one another in sequence. In some cases, the sample includes 10 or more, 20 or more, 50 or more, 100 or more, 500 or more, 10³ or more, 5×10³ or more, 10⁴ or more, 5×10⁴ or more, 10⁵ or more, 5×10⁵ or more, 10⁶ or more 5×10⁶ or more, or 10⁷ or more, RNAs or DNAs. In some cases, the sample comprises from 10 to 20, from 20 to 50, from 50 to 100, from 100 to 500, from 500 to 10³, from 10³ to 5×10³, from 5×10³ to 10⁴, from 10⁴ to 5×10⁴, from 5×10⁴ to 10⁵, from 10⁵ to 5×10⁵, from 5×10⁵ to 10⁶, from 10⁶ to 5×10⁶, or from 5×10⁶ to 10⁷, or more than 10⁷, RNAs or DNAs. In some cases, the sample comprises from 5 to 10⁷ RNAs or DNAs (e.g., that differ from one another in sequence) (e.g., from 5 to 10⁶, from 5 to 10⁵, from 5 to 50,000, from 5 to 30,000, from 10 to 10⁶, from 10 to 10⁵, from 10 to 50,000, from 10 to 30,000, from 20 to 10⁶, from 20 to 10⁵, from 20 to 50,000, or from 20 to 30,000 RNAs or DNAs). In some cases, the sample includes 20 or more RNAs or DNAs that differ from one another in sequence. In some cases, the sample includes RNAs or DNAs from a cell lysate (e.g., a eukaryotic cell lysate, a mammalian cell lysate, a human cell lysate, a prokaryotic cell lysate, a plant cell lysate, and the like). For example, in some cases the sample includes RNA or DNA from a cell such as a eukaryotic cell, e.g., a mammalian cell such as a human cell.

Suitable samples include but are not limited to saliva, blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., DNAs. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising DNAs that is obtained from such cells (e.g., a cell lysate or other cell extract comprising DNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell, tissue, or organ; a cell, tissue, or organ from an invertebrate animal (e.g., fruit fly, cnidarian, echinoderm, nematode, an insect, an arachnid, etc.); a cell, tissue, fluid, or organ from a vertebrate animal (e.g., fish, amphibian, reptile, bird, mammal); a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms, e.g., Bacteria (e.g., Eubacteria); Archaebacteria; Protista; Fungi; Plantae; and Animalia. Suitable sample sources include plant-like members of the kingdom Protista, including, but not limited to, algae (e.g., green algae, red algae, glaucophytes, cyanobacteria); fungus-like members of Protista, e.g., slime molds, water molds, etc; animal-like members of Protista, e.g., flagellates (e.g., Euglena), amoeboids (e.g., amoeba), sporozoans (e.g., Apicomplexa, Myxozoa, Microsporidia), and ciliates (e.g., Paramecium). Suitable sample sources include members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable sample sources include members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable sample sources include members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms)p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g., starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Ayes (birds); and Mammalian (mammals) Suitable plants include any monocotyledon and any dicotyledon.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some cases, the source of the sample is a (or is suspected of being a diseased cell, fluid, tissue, or organ, for example of a human subject. In some cases, the source of the sample is a normal (non-diseased) cell, fluid, tissue, or organ. In some cases, the source of the sample is a (or is suspected of being a pathogen-infected cell, tissue, or organ. For example, the source of a sample can be an individual who may or may not be infected—and the sample could be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. In some cases, the sample is a cell-free liquid sample. In some cases, the sample is a liquid sample that can comprise cells.

Pathogens to be detected in samples include viruses, bacteria, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., coronaviruses (e.g., COVID-19, MERS, SARS, etc.); immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogenic viruses can include DNA viruses such as: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Pathogens can include, e.g., DNAviruses [e.g.: a papovavirus (e.g., human papillomavirus (HPV), polyomavirus); a hepadnavirus (e.g., Hepatitis B Virus (HBV)); a herpesvirus (e.g., herpes simplex virus (HSV), varicella zoster virus (VZV), epstein-barr virus (EBV), cytomegalovirus (CMV), herpes lymphotropic virus, Pityriasis rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like], Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

### Kits

The present disclosure provides a kit for detecting a target nucleotide sequences, e.g., in a sample comprising a plurality of sequences. In some cases, the kit comprises one or more activators, compositions or systems as described herein. Positive and/or negative controls may also be included and/or instructions for use may also be included.

### EXAMPLES

### Example 1: Modifications of Type III Accessory Nuclease Activators

The data presented here are also shown in Liu et al. (Nature Chemical Biology, 17:982-988 (2021)), the contents of which are incorporated by reference herein in their entirety.

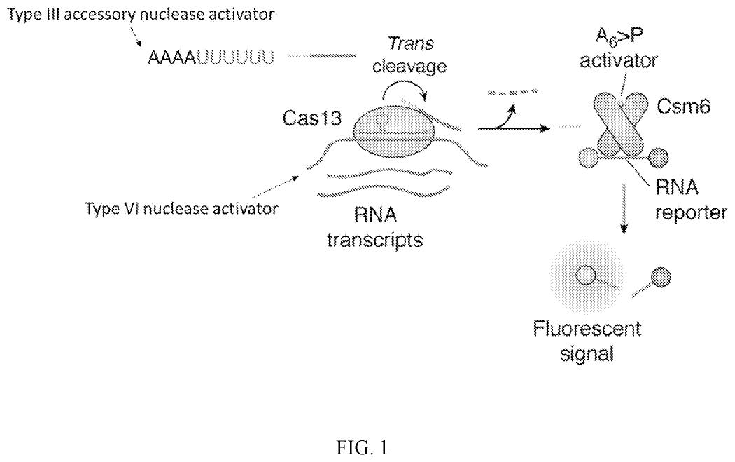

To test the impact of modifications of the Type III accessory nuclease activators, the following system was used (see FIG. 1). Cas13 ribonucleoprotein complexes (RNP) were assembled using LbuCas13 protein and guides at room temperature at a concentration of 0.5 μM Cas13 in 1× reaction buffer. The guides used in the experiments described below are shown in Table 2.

TABLE 2
Guides and Type VI nuclease Activators

| | | Guide or | | SEQ |
|---|---|---|---|---|
| Name | Gene Target | Activator? | Sequence | ID NO |
| R010 | Synthetic | Cas13 | AUUUAGACCACCCCAAAAAUGAAGGGGACUAAAACACAGAUAUAGCCU | 3 |
| | | Guide | GGUGGUUCAGGC | |
| R004 | Short RNA target for | Cas13 | AAAAGCCUGAACCACCAGGCUAUAUCUG | 4 |
| | R100 | activator | | |
| GJK073 | First spacer from the | Cas13 | UAGACCAGCCCAAAAAUGAAGGGCACUAAAACGCAGCGCCUCUUGCAA | 5 |
| | CRISPR array of | guide | CGAUUAAAUACU | |
| | Lachnospriaceae | | | |
| | bacterium genome. | | | |
| GJK075 | Short RNA target for | Cas13 | AGUAUUUAAUCGUUGCAAGAGGCGCUGCUC | 6 |
| | GJK073 | activator | | |
| NCR316 | Orf1a of the SARS-CoV-2 | Cas13 | UAGACCACCCCAAAAAUGAAGGGGACUAAAACGUUCGGACAAAGUGCA | 7 |
| | genome. | guide | UGAA | |
| aNCR316 | Short RNA target for | Cas13 | UAAAGCUUCAUGCACUUUGUCCGAACAACUGG | 8 |
| | NCR316 corresponding to | activator | | |
| | Orf1a of the SARS-CoV-2 | | | |
| | genome. | | | |
| NCR604 | N gene of the SARS-CoV-2 | Guide | uagaccaccccaaaaaugaaggggacuaaaacGGUCCACCAAACGUAA | 9 |
| | genome | | UGCG | |
| NCR612 | N gene of the SARS-CoV-2 | Guide | uagaccaccccaaaaaugaaggggacuaaaacUUUGCGGCCAAUGUUU | 10 |
| | genome | | GUAA | |
| Gblock | Fragment of the | | CAGGUUACUAUAGCAGAGAUAUUACUAAUUAUUAUGAGGACUUUUAAA | 11 |
| 7 | SARS-CoV2-genome | | GUUUCCAUUUGGAAUCUUGAUUACAUCAUAAACCUCAUAAUUAAAAAU | |
| | (includes part of the | | UUAUCUAAGUCACUAACUGAGAAUAAAUAUUCUCAAUUAGAUGAAGAG | |
| | ORF6 gene, and the | | CAACCAAUGGAGAUUGAUUAAACGAACAUGAAAAUUAUUCUUUUCUUG | |
| | complete ORF7a, ORF7b, | | GCACUGAUAACACUCGCUACUUGUGAGCUUUAUCACUACCAAGAGUGU | |
| | ORF8, N, and ORF10 | | GUUAGAGGUACAACAGUACUUUUAAAAGAACCUUGCUCUUCUGGAACA | |
| | genes). | | UACGAGGGCAAUUCACCAUUUCAUCCUCUAGCUGAUAACAAAUUUGCA | |
| | | | CUGACUUGCUUUAGCACUCAAUUUGCUUUUGCUUGUCCUGACGGCGUA | |
| | | | AAACACGUCUAUCAGUUACGUGCCAGAUCAGUUUCACCUAAACUGUUC | |
| | | | AUCAGACAAGAGGAAGUUCAAGAACUUUACUCUCCAAUUUUUCUUAUU | |
| | | | GUUGCGGCAAUAGUGUUUAUAACACUUUGCUUCACACUCAAAAGAAAG | |
| | | | ACAGAAUGAUUGAACUUUCAUUAAUUGACUUCUAUUUGUGCUUUUUAG | |
| | | | CCUUUCUGCUAUUCCUUGUUUUAAUUAUGCUUAUUAUCUUUUGGUUCU | |
| | | | CACUUGAACUGCAAGAUCAUAAUGAAACUUGUCACGCCUAAACGAACA | |
| | | | UGAAAUUUCUUGUUUUCUUAGGAAUCAUCACAACUGUAGCUGCAUUUC | |
| | | | ACCAAGAAUGUAGUUUACAGUCAUGUACUCAACAUCAACCAUAUGUAG | |
| | | | UUGAUGACCCGUGUCCUAUUCACUUCUAUUCUAAAUGGUAUAUUAGAG | |
| | | | UAGGAGCUAGAAAAUCAGCACCUUUAAUUGAAUUGUGCGUGGAUGAGG | |
| | | | CUGGUUCUAAAUCACCCAUUCAGUACAUCGAUAUCGGUAAUUAUACAG | |
| | | | UUUCCUGUUUACCUUUUACAAUUAAUUGCCAGGAACCUAAAUUGGGUA | |
| | | | GUCUUGUAGUGCGUUGUUCGUUCUAUGAAGACUUUUUAGAGUAUCAUG | |
| | | | ACGUUCGUGUUGUUUUAGAUUUCAUCUAAACGAACAAACUAAAAUGUC | |
| | | | UGAUAAUGGACCCCAAAAUCAGCGAAAUGCACCCCGCAUUACGUUUGG | |
| | | | UGGACCCUCAGAUUCAACUGGCAGUAACCAGAAUGGAGAACGCAGUGG | |
| | | | GGCGCGAUCAAAACAACGUCGGCCCCAAGGUUUACCCAAUAAUACUGC | |
| | | | GUCUUGGUUCACCGCUCUCACUCAACAUGGCAAGGAAGACCUUAAAUU | |
| | | | CCCUCGAGGACAAGGCGUUCCAAUUAACACCAAUAGCAGUCCAGAUGA | |
| | | | CCAAAUUGGCUACUACCGAAGAGCUACCAGACGAAUUCGUGGUGGUGA | |
| | | | CGGUAAAAUGAAAGAUCUCAGUCCAAGAUGGUAUUUCUACUACCUAGG | |
| | | | AACUGGGCCAGAAGCUGGACUUCCCUAUGGUGCUAACAAAGACGGCAU | |
| | | | CAUAUGGGUUGCAACUGAGGGAGCCUUGAAUACACCAAAAGAUCACAU | |
| | | | UGGCACCCGCAAUCCUGCUAACAAUGCUGCAAUCGUGCUACAACUUCC | |
| | | | UCAAGGAACAACAUUGCCAAAAGGCUUCUACGCAGAAGGGAGCAGAGG | |
| | | | CGGCAGUCAAGCCUCUUCUCGUUCCUCAUCACGUAGUCGCAACAGUUC | |
| | | | AAGAAAUUCAACUCCAGGCAGCAGUAGGGGAACUUCUCCUGCUAGAAU | |
| | | | GGCUGGCAAUGGCGGUGAUGCUGCUCUUGCUUUGCUGCUGCUUGACAG | |
| | | | AUUGAACCAGCUUGAGAGCAAAAUGUCUGGUAAAGGCCAACAACAACA | |
| | | | AGGCCAAACUGUCACUAAGAAAUCUGCUGCUGAGGCUUCUAAGAAGCC | |
| | | | UCGGCAAAAACGUACUGCCACUAAAGCAUACAAUGUAACACAAGCUUU | |
| | | | CGGCAGACGUGGUCCAGAACAAACCCAAGGAAAUUUUGGGGACCAGGA | |
| | | | ACUAAUCAGACAAGGAACUGAUUACAAACAUUGGCCGCAAAUUGCACA | |
| | | | AUUUGCCCCCAGCGCUUCAGCGUUCUUCGGAAUGUCGCGCAUUGGCAU | |
| | | | GGAAGUCACACCUUCGGGAACGUGGUUGACCUACACAGGUGCCAUCAA | |
| | | | AUUGGAUGACAAAGAUCCAAAUUUCAAAGAUCAAGUCAUUUUGCUGAA | |
| | | | UAAGCAUAUUGACGCAUACAAAACAUUCCCACCAACAGAGCCUAAAAA | |
| | | | GGACAAAAAGAAGAAGGCUGAUGAAACUCAAGCCUUACCGCAGAGACA | |
| | | | GAAGAAACAGCAAACUGUGACUCUUCUUCCUGCUGCAGAUUUGGAUGA | |
| | | | UUUCUCCAAACAAUUGCAACAAUCCAUGAGCAGUGCUGACUCAACUCA | |
| | | | GGCCUAAACUCAUGCAGACCACACAAGGCAGAUGGGCUAUAUAAACGU | |
| | | | UUUCGCUUUUCCGUUUACGAUAUAUAGUCUACUCUUGUGCAGAAUGAA | |
| | | | UUCUCGUAACUACAUAGCACAAGUAGAUGUAGUUAACUUUAAUCUCAC | |
| | | | AUAGCAAUCUUUAAUCAGUGUGUAACAUUAGGGAGGACUUGAAAGAGC | |
| | | | CACCACAUUUUCACCGAGGCCACGCGGAGUACGAUCGAGUGUACAGUG | |
| | | | AACAAUGCUAGGGAGAGCUGCCUAUAUGGAAGAGCCCUAAUGUGUAAA | |
| | | | AUUAAUUUUAGUAGUGCUAUCCCCAUGUGAUUUUAAUAGCUUCUUAGG | |
| | | | AGAAUGACAAAAAAAAAAAAAAAAAAAA | |

In some experiments, the 1× reaction buffer comprised 20 mM HEPES, pH 6.8, 50 mM KCl, 5 mM MgCl₂, 100 μg/mL BSA, 0.01% Igepal CA, 2% glycerol (“IGI buffer”) while in other experiments, the 1× reaction buffer comprised 20 mM HEPES, pH 6.8, 50 mM KCl, 5 mM MgCl₂, 5% glycerol (“Ott buffer”). Once assembled, the RNPs were diluted with 1× reaction buffer to 10× the final [RNP] in the reaction (200 nM for 20 nM final concentration). Concentrated T. thermophilus Csm6 (TtCsm6) protein was also diluted to 10× the concentration (1 μM for 100 nM final concentration) in 1× reaction buffer. The activator mix was assembled on ice, containing 20× concentration of the Cas13 activator, 20× concentration of the secondary Csm6 activator, and 10× of a polyC RNA reporter (6-FAM-CCCCC-Iowa Black®, Integrated DNA Technologies) in 1× reaction buffer. The proteins were then mixed together to make an RNP/protein master mix in 1× reaction buffer. Volumes to use were determined such that 15 μL of this master mix was added to 5 μL of the activator mix for a final volume of 20 μL for the reaction. Reactions were performed in triplicate. The RNP master mix was equilibrated at room temperature for 10-15 minutes. 15 μL of the RNP/protein master mix was then added to 5 μL of the activator master mix (pre-loaded into 384-well low-volume flat-bottom black assay plate, Corning) to start the reaction. Fluorescence of the unquenched 6-FAM group (excitation 485 nm, emission 535 or 528 nm) was monitored over 1-2 hours (one reading every 1.5 or 2 min) in a Tecan or Biotek Cytation 5 plate reader at 37° C. An increase in fluorescence correlates with dequenching of the FAM due to RNA cleavage by TtCsm6.

Three types of modified Type III accessory nuclease activators were tested: activators comprising 2′OMe modification, activators comprising 2′deoxy modification and activators comprising 2′-fluoro modification. Exemplary activators used in these experiments are shown below in Table 3.

TABLE 3
Exemplary Type III Accessory Nuclease Activators

| | | | SEQ |
|---|---|---|---|
| Name | Modification Type | Sequence | ID NO |
| NCR142 | unmodified | AAAAUUUUUU | 1 |
| NCR372 | 2′-OMe | mAmAmAAUUUUUU | 12 |
| NCR373 | 2′-deoxy | dAdAdAAUUUUUU | 13 |
| NCR470 | 2′-deoxy | AdAAAUUUUUU | 14 |
| NCR655 | 2′-deoxy | dAAAAUUUUUU | 15 |
| NCR654 | 2′-deoxy | AAdAAUUUUUU | 16 |
| NCR657 | 2′-deoxy | AdAdAAUUUUUU | 17 |
| NCR656 | 2′-deoxy | dAdAAAUUUUUU | 18 |
| NCR374 | 2′-fluoro | fAfAfAAUUUUUU | 19 |
| NCR497 | 2′-fluoro | AfAAAUUUUUU | 20 |
| NCR690 | 2′-fluoro | AfAAAUCC (metdC) | 2 |
| | | (metdc) C | |

The Type III accessory nuclease activators were tested as follows.

NCR142, NCR372, NCR373 and NCR374 were compared in the system described above. Specifically, 20 nM Cas13 RNP (assembled at a ratio of 2:1 guide to protein), 100 nM Csm6, 100 pM Cas13 activator [R010]], 200 nM C5 RNA reporter (i.e., 5′-6-FAM-CCCCC-Iowa Black-3′, Integrated DNA Technologies), 2 μM Type III accessory nuclease activator, 1×IGI buffer were used, with the experiment monitored on a Tecan Spark.

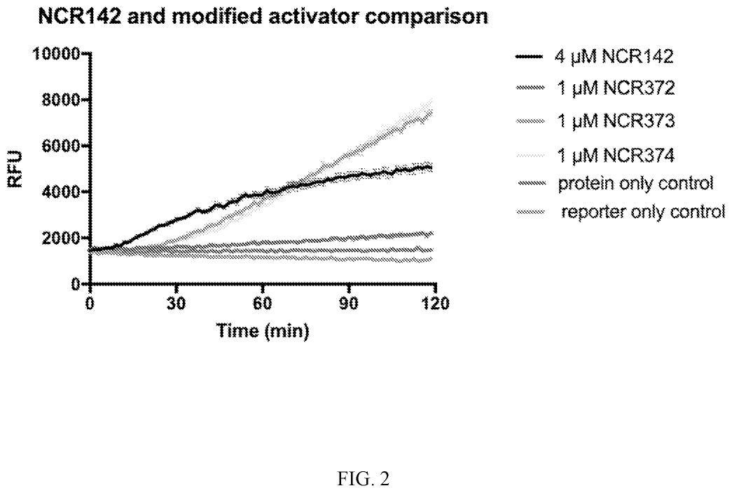

The results (as shown in FIG. 2) demonstrated that all Type III accessory nuclease activators had activity although the modified activators were more active than the unmodified activator.

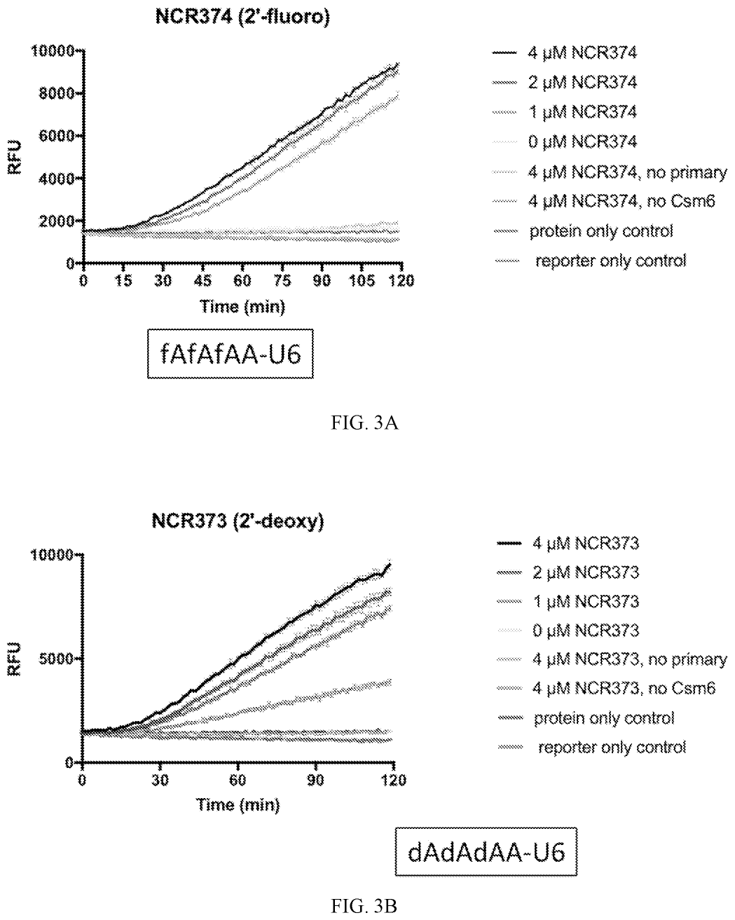

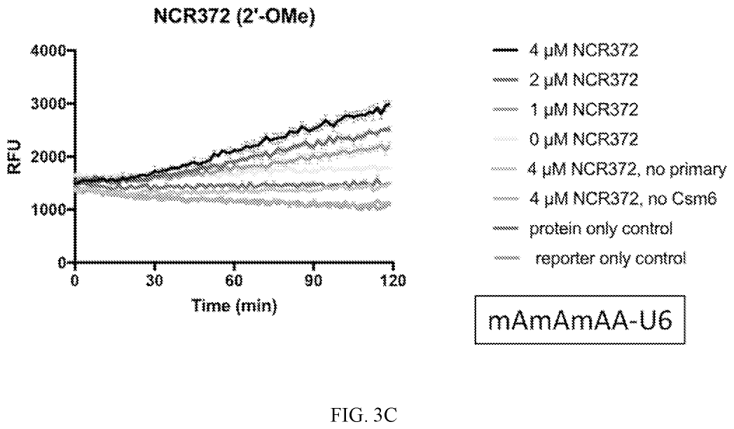

The Type III accessory nuclease activators were also tested over a range of concentrations. As shown in FIG. 3), the 2′-fluoro and 2′-deoxy modified activators were more active than the 2′OMe modified activator.

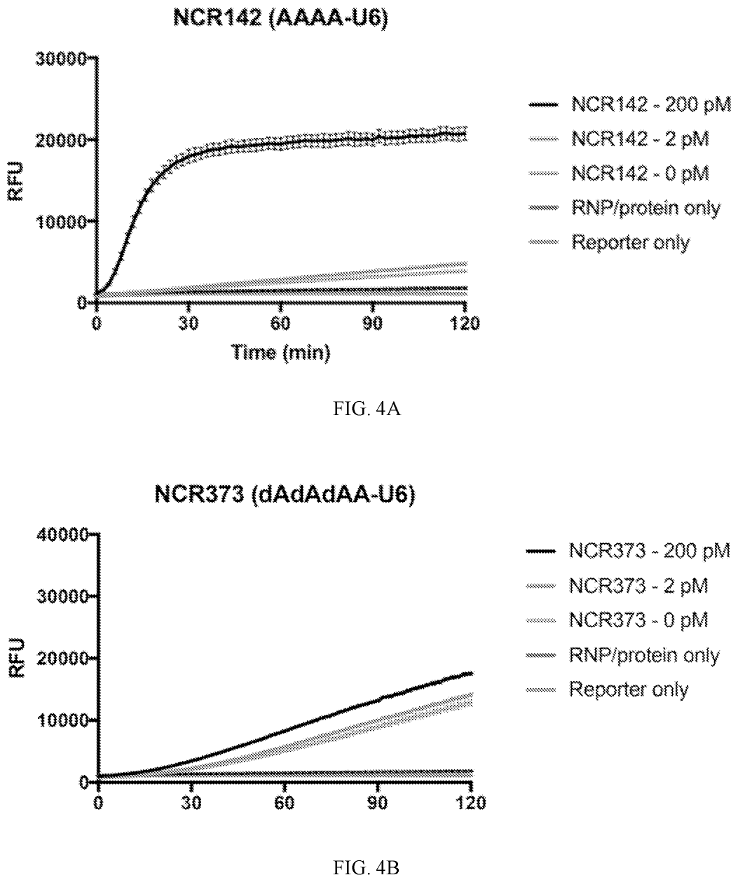

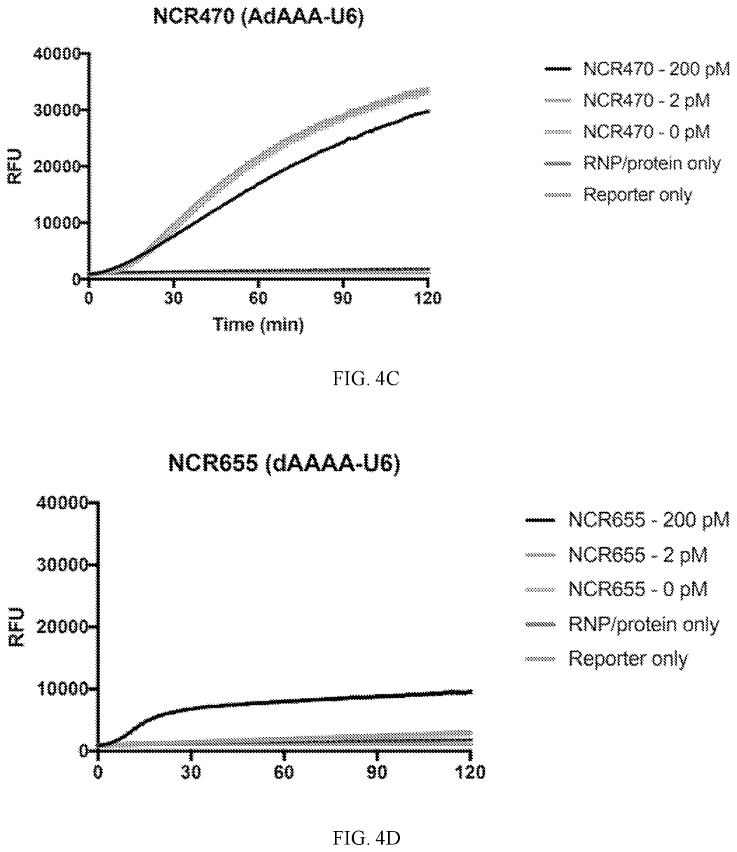

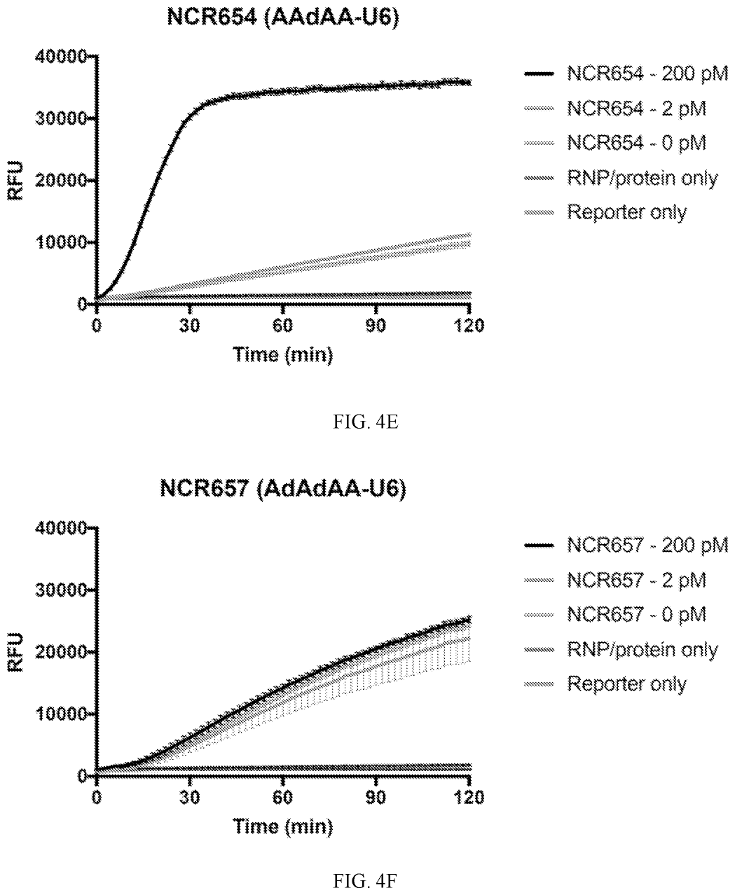

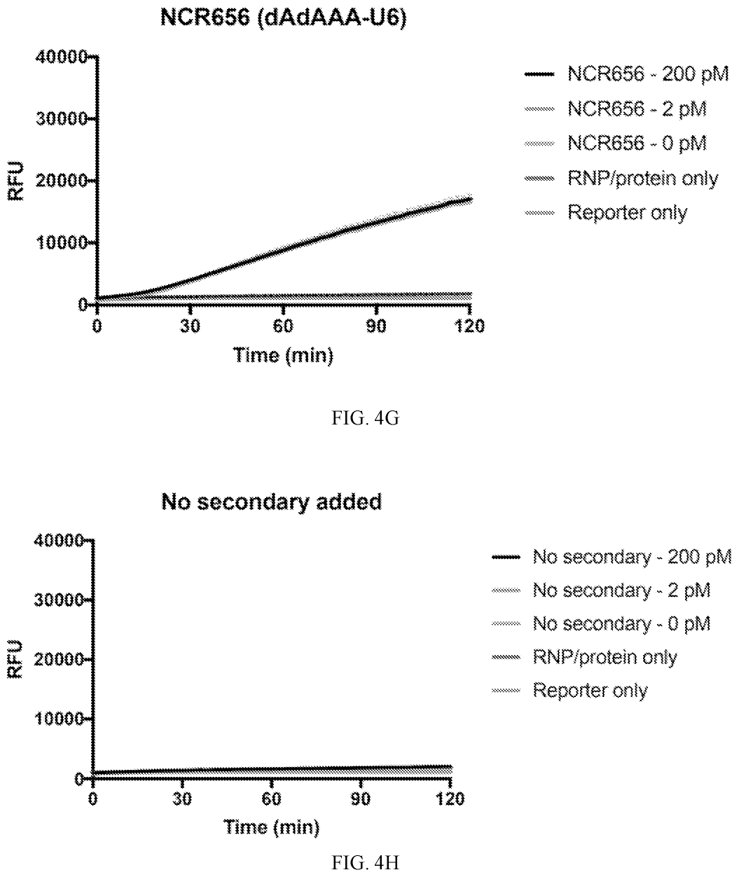

Next, several different 2′-deoxy modified Type III accessory nuclease activators were compared where the activators had 1 to 3 modifications and the modifications were in different locations (see Table 3 and FIG. 4). In these experiments, 20 nM Cas13 RNPs using a guide, GJK073, targeting a short synthetic RNA target, GJK075, was used (see Table 2). In addition, 100 nM Csm6 was used, plus 0 to 200 pM Cas13 activator (GJK075), 2 μM Csm6 activator, 200 nM C5 reporter, and Cas13 1× buffer (IGI). The experiments were carried out at 37° C. in low volume flat-bottom black plates and fluorescence was measured on a Biotek Cytation 5.

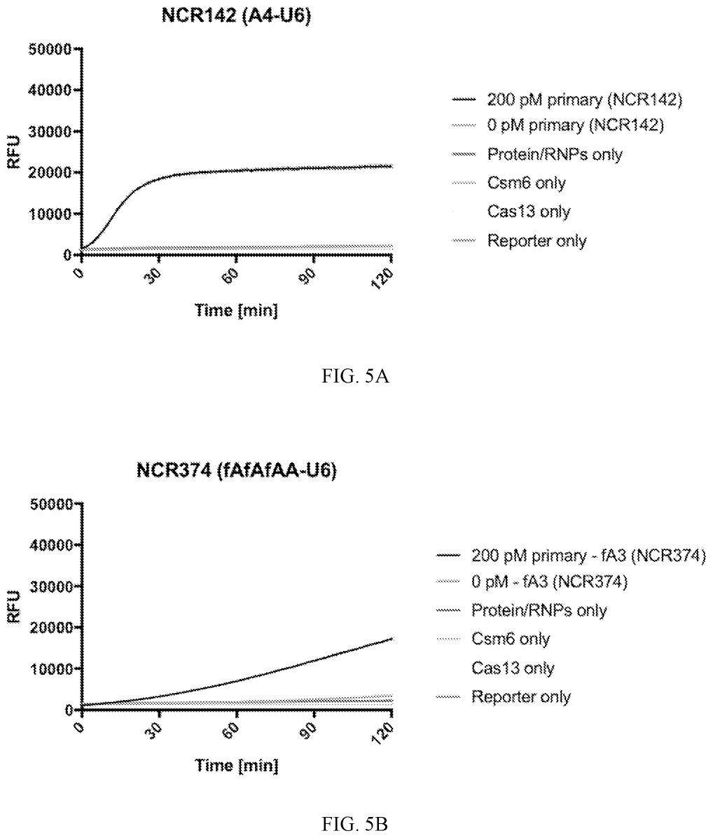

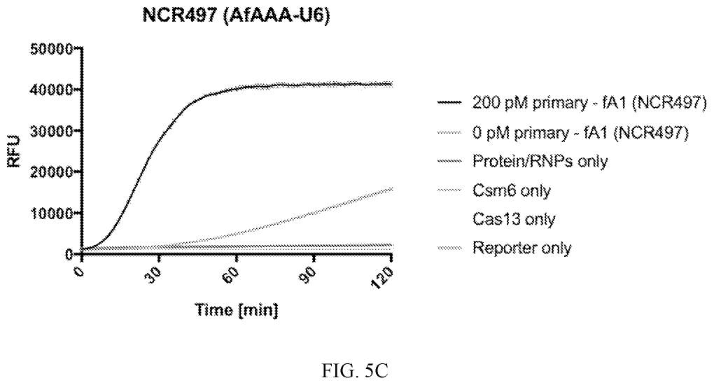

As shown in FIG. 4, the results demonstrated that some of the modified Type III accessory nuclease activators had rapid kinetics and were able to achieve a high and stable signal (for example NCR654) as compared with unmodified activators. Two modified Type III accessory nuclease activators comprising 2′-fluoro modifications were also tested. One activator comprised three 2′-fluoro modified nucleotides (NCR374) while the second had a single 2′-fluoro modified nucleotide (NCR497). The results (FIG. 5) demonstrate rapid and stable kinetics using the NCR497 activator with decreased background signal (0 pM Cas13 activator curves) as compared to the 2′-deoxy modified Type III accessory nuclease activators.

The NCR497 activator was also tested over a range of Type III accessory nuclease activator concentrations and Type VI nuclease activator concentrations. In these experiments, 20 nM Cas13 RNPs comprising the NCR316 guide at a ratio of 2:1 protein to guide were used. In addition, 100 nM TtCsm6 and 200 nM C5 reporter was used where the reaction was carried out in low-volume, flat-bottom plates at 37° C. analyzed on a Biotek citation 5. Type VI nuclease activator (aNCR316) was used at 2 pM or 200 pM concentrations.

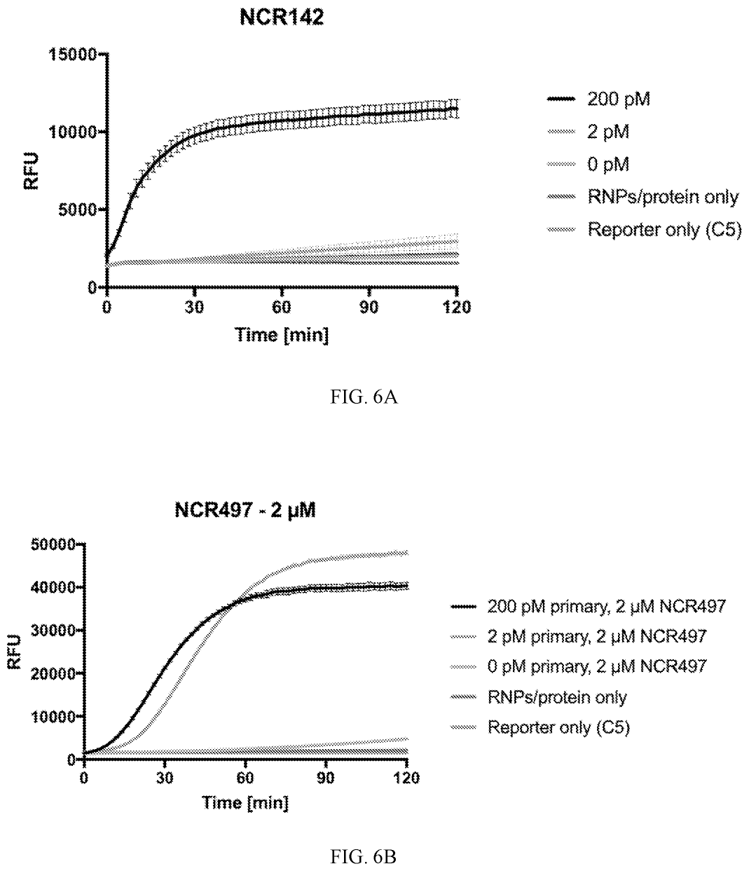

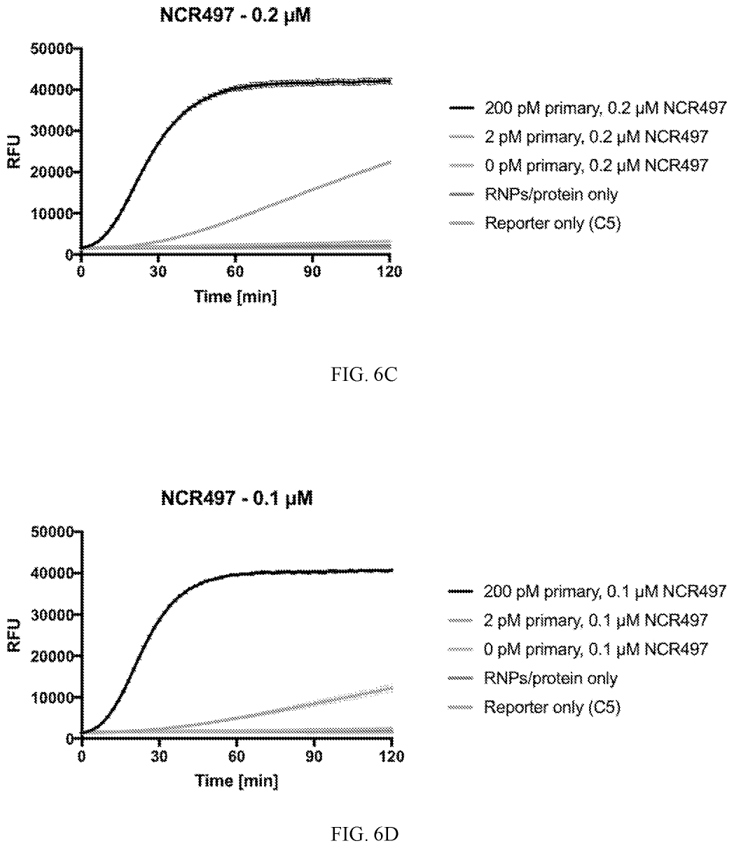

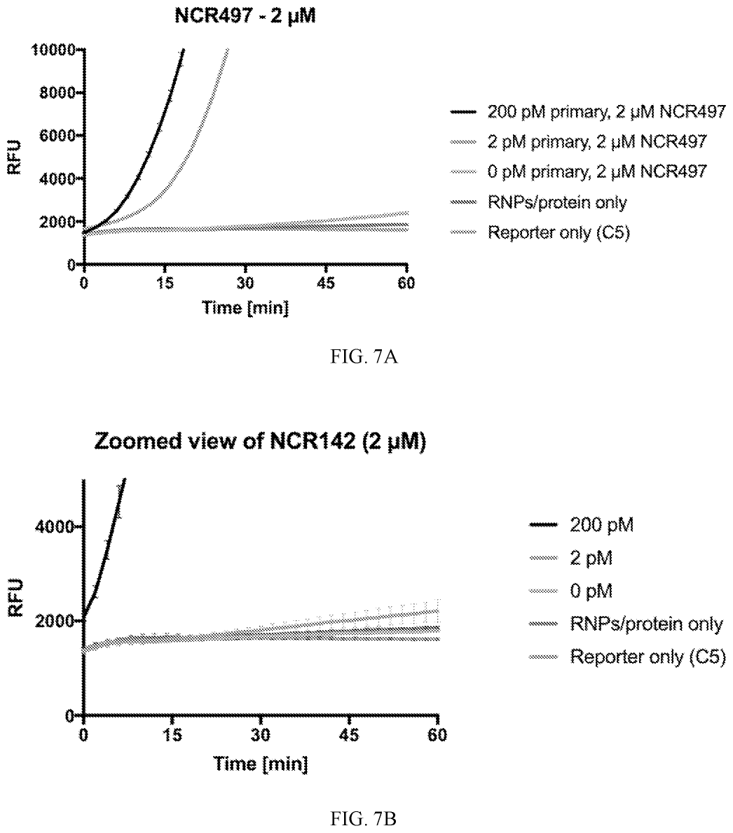

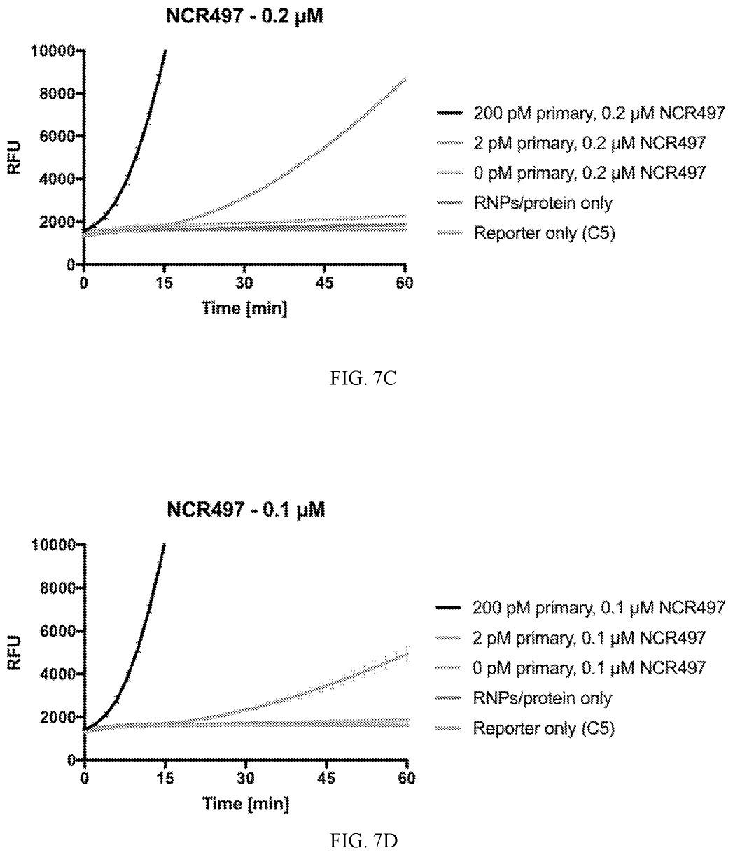

As shown in FIGS. 6 and 7, the results demonstrated that NCR497 gave better signal in the reaction at 2 pM of Type VI nuclease activator than the unmodified NCR142 Type III accessory nuclease activator using either 2 pM or 200 pM of Type VI nuclease activator.

A Type III accessory nuclease activator comprising a single 2′-fluoro modified nucleotide was also modified to create an activator with a single cleavage location for the Cas13 trans nuclease activity. In this Type III accessory nuclease activator, the poly U stretch was replaced with a single U nucleotide followed by C nucleotides. Two of the C nucleotides had modifications to their bases, i.e., 5-methylcytosine, so as to avoid competition of the cleaved tail with cytidines in the fluorescence reporter for Csm6 recognition and cleavage. This activator (NCR690) was tested in the same manner as the NCR497 activator.

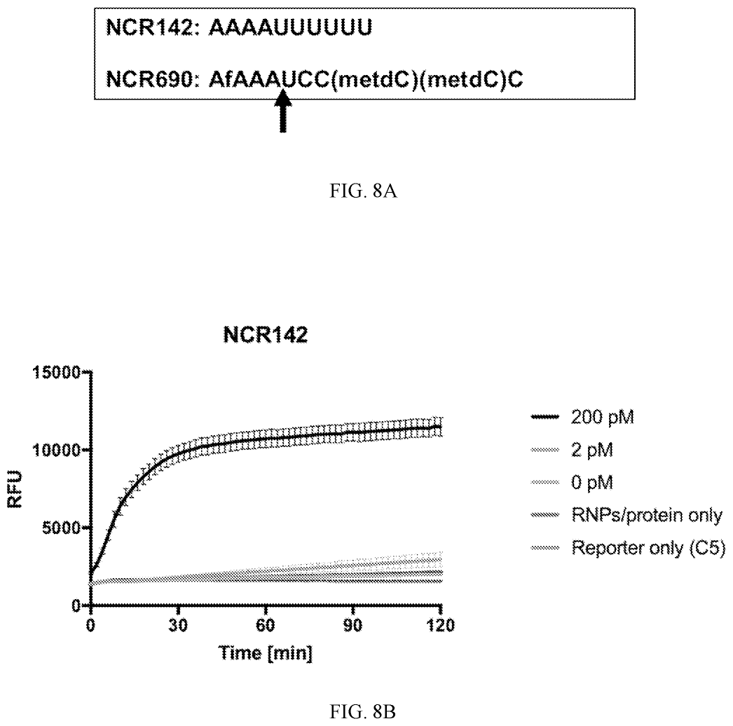

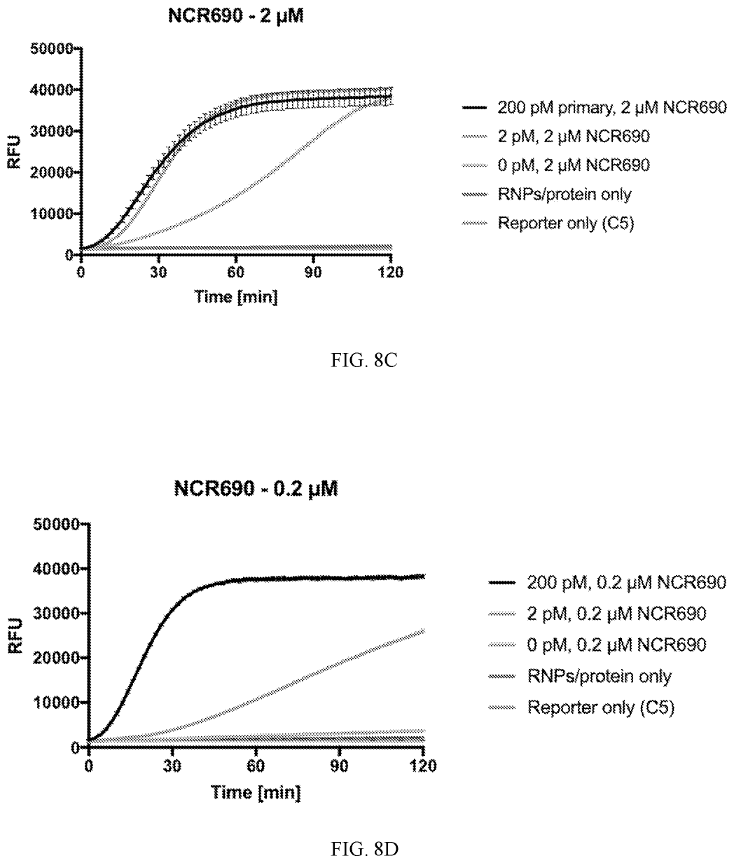

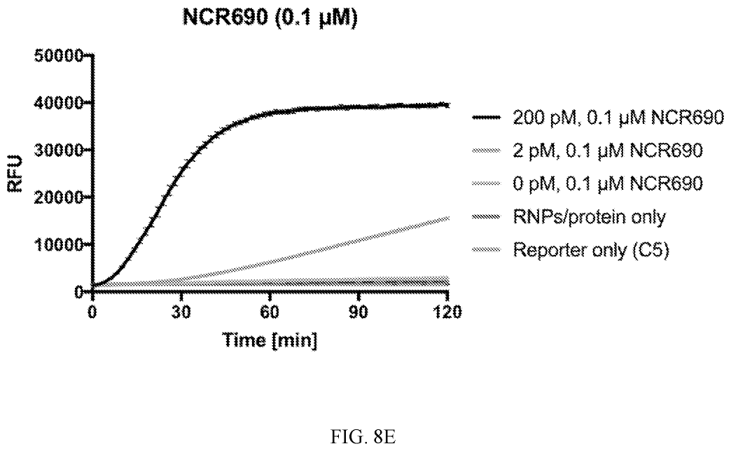

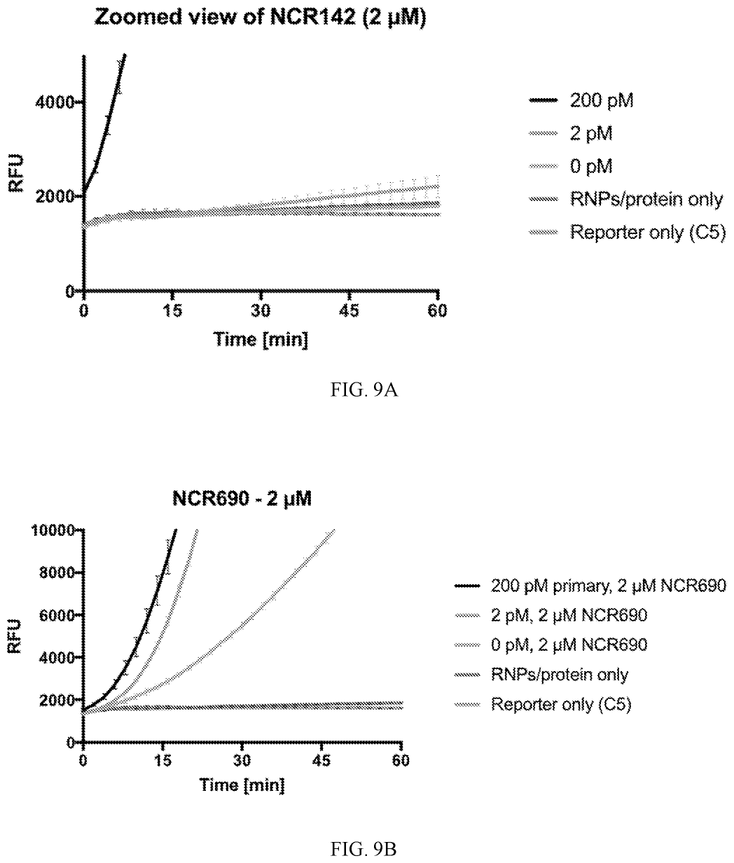

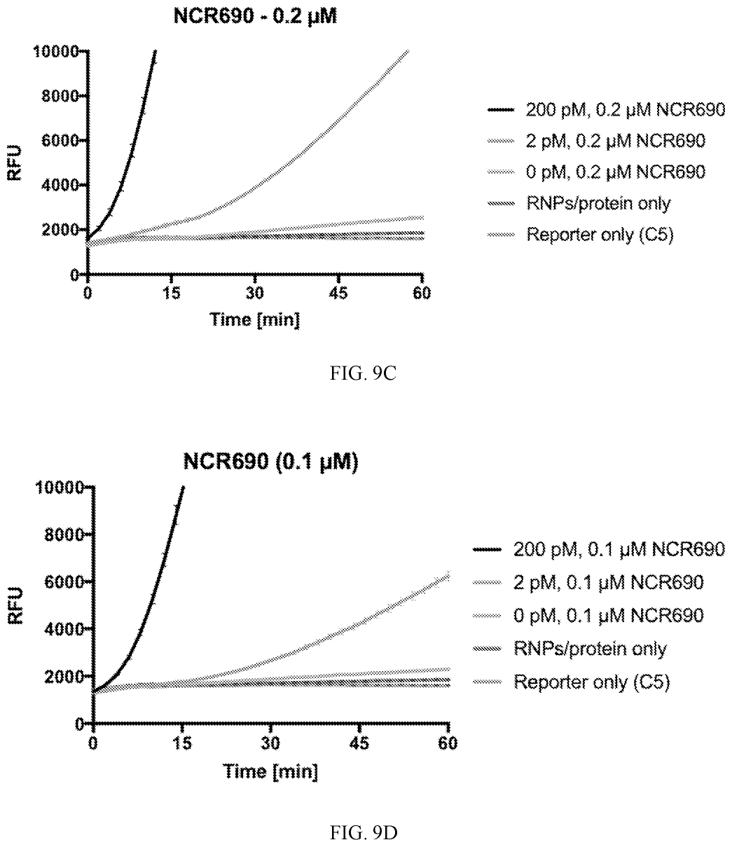

As shown in FIGS. 8 and 9, the results demonstrated that the system using the NCR690 activator also had robust activity when it was used at 2, 0.2 and 0.1 μM concentration and the Cas13 activator was used at either 200 or 2 pM concentration. NCR690 appeared to have higher background signal compared to the NCR497 when used at the same concentrations.

The system was then assayed for activity in the presence or absence of Csm6 and its Type III accessory nuclease activator. In these experiments, 200 nM Cas13 RNP comprising the NCR316 guide was compared with the same RNPs where the reaction further comprised 100 nM Csm6 and 2 μM NCR497 activator. Both reactions also contained 200 nM C5 reporter where the Type VI nuclease activator aNCR316 was used in concentrations ranging from 200 aM to 200 pM. The reactions were carried out at 37° C. in 1×IGI buffer.

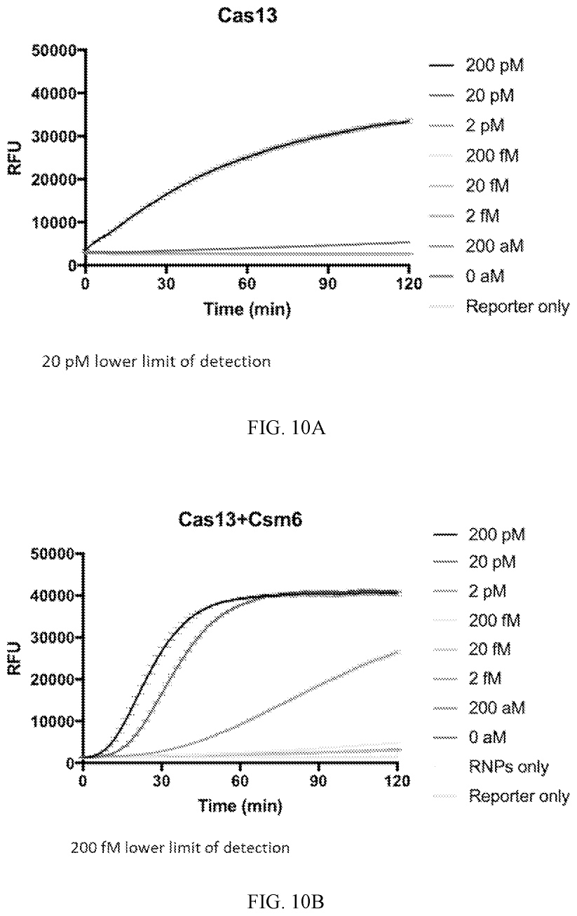

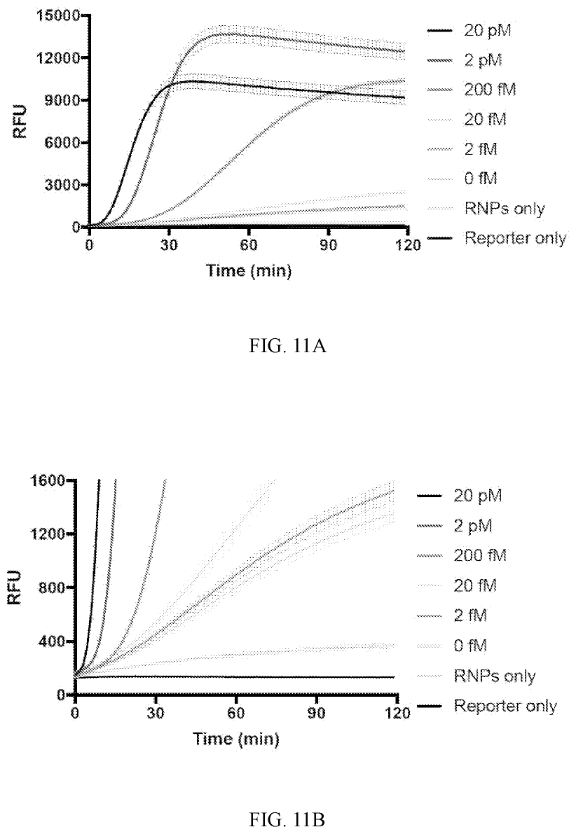

As shown in FIG. 10, a 100-fold increase in sensitivity was achieved when the Csm6 reagents were added to the Cas13 detection system. These experiments were repeated using another set of guide RNAs NCR604 and NCR612 in a different buffer system. The Type VI nuclease activator in this experiment was the gblock7 transcript (Table 2). 50 nM Cas13 RNP comprising equal proportions of guides NCR604 and NCR612 were used with 100 nM Csm6, 2 μM NCR497, 200 nM C5 reporter, where the concentration of Type VI nuclease activator was varied from 0-20 pM. The reaction was carried out in Ott buffer at 37° C. A short room temperature pre-equilibration step (˜15 min) of the components was also included before the assay was started and demonstrated that in this experimental system, the limit of detection of the Cas13 activator was approximately 2 fM. See, FIG. 10.

Thus, the Type III accessory nucleases activators described herein unexpectedly increase the sensitivity and/or efficiency of detection of nucleic acids in a sample, including when used in conjunction with Type VI Cas protein detection systems.

### Example 2: Sequences of Proteins that May be Used with the Methods and Compositions of the Invention

Cas13a sequences LshCas13a, WP_018451595.1 (Abudayyeh, O. O., (2016)
Science 353 (6299), aaf5573)
(SEQ ID NO: 21)

| | |
|---|---|
| 1 | MGNLFGHKRW YEVRDKKDFK IKRKVKVKRN YDGNKYILNI NENNNKEKID NNKFIRKYIN |
| 61 | YKKNDNILKE FTRKFHAGNI LFKLKGKEGI IRIENNDDFL ETEEVVLYIE AYGKSEKLKA |
| 121 | LGITKKKIID EAIRQGITKD DKKIEIKRQE NEEEIEIDIR DEYTNKTLND CSIILRIIEN |
| 181 | DELETKKSIY EIFKNINMSL YKIIEKIIEN ETEKVFENRY YEEHLREKLL KDDKIDVILT |
| 241 | NFMEIREKIK SNLEILGFVK FYLNVGGDKK KSKNKKMLVE KILNINVDLT VEDIADEVIK |
| 301 | ELEFWNITKR IEKVKKVNNE FLEKRRNRTY IKSYVLLDKH EKFKIERENK KDKIVKFFVE |
| 361 | NIKNNSIKEK IEKILAEFKI DELIKKLEKE LKKGNCDTEI FGIFKKHYKV NFDSKKFSKK |
| 421 | SDEEKELYKI IYRYLKGRIE KILVNEQKVR LKKMEKIEIE KILNESILSE KILKRVKQYT |
| 481 | LEHIMYLGKL RHNDIDMTTV NTDDFSRLHA KEELDLELIT FFASTNMELN KIFSRENINN |
| 541 | DENIDFFGGD REKNYVLDKK ILNSKIKIIR DLDFIDNKNN ITNNFIRKFT KIGTNERNRI |
| 601 | LHAISKERDL QGTQDDYNKV INIIQNLKIS DEEVSKALNL DVVFKDKKNI ITKINDIKIS |
| 661 | EENNNDIKYL PSFSKVLPEI LNLYRNNPKN EPFDTIETEK IVLNALIYVN KELYKKLILE |
| 721 | DDLEENESKN IFLQELKKTL GNIDEIDENI IENYYKNAQI SASKGNNKAI KKYQKKVIEC |
| 781 | YIGYLRKNYE ELFDFSDFKM NIQEIKKQIK DINDNKTYER ITVKTSDKTI VINDDFEYII |
| 841 | SIFALLNSNA VINKIRNRFF ATSVWLNTSE YQNIIDILDE IMQLNTLRNE CITENWNLNL |
| 901 | EEFIQKMKEI EKDFDDFKIQ TKKEIFNNYY EDIKNNILTE FKDDINGCDV LEKKLEKIVI |
| 961 | FDDETKFEID KKSNILQDEQ RKLSNINKKD LKKKVDQYIK DKDQEIKSKI LCRIIFNSDF |
| 1021 | LKKYKKEIDN LIEDMESENE NKFQEIYYPK ERKNELYIYK KNLFLNIGNP NFDKIYGLIS |
| 1081 | NDIKMADAKF LFNIDGKNIR KNKISEIDAI LKNLNDKLNG YSKEYKEKYI KKLKENDDFF |
| 1141 | AKNIQNKNYK SFEKDYNRVS EYKKIRDLVE FNYLNKIESY LIDINWKLAI QMARFERDMH |
| 1201 | YIVNGLRELG IIKLSGYNTG ISRAYPKRNG SDGFYTTTAY YKFFDEESYK KFEKICYGFG |
| 1261 | IDLSENSEIN KPENESIRNY ISHFYIVRNP FADYSIAEQI DRVSNLLSYS TRYNNSTYAS |
| 1321 | VFEVFKKDVN LDYDELKKKF KLIGNNDILE RLMKPKKVSV LELESYNSDY IKNLIIELLT |
| 1381 | KIENTNDTL |

LwaCas13a, WP_021746774.1, hypothetical protein
(SEQ ID NO: 22)

| | |
|---|---|
| 1 | MKVTKVDGIS HKKYIEEGKL VKSTSEENRT SERLSELLSI RLDIYIKNPD NASEEENRIR |
| 61 | RENLKKFFSN KVLHLKDSVL YLKNRKEKNA VQDKNYSEED ISEYDLKNKN SFSVLKKILL |
| 121 | NEDVNSEELE IFRKDVEAKL NKINSLKYSF EENKANYQKI NENNVEKVGG KSKRNIIYDY |
| 181 | YRESAKRNDY INNVQEAFDK LYKKEDIEKL FFLIENSKKH EKYKIREYYH KIIGRKNDKE |
| 241 | NFAKIIYEEI QNVNNIKELI EKIPDMSELK KSQVFYKYYL DKEELNDKNI KYAFCHFVEI |
| 301 | EMSQLLKNYV YKRLSNISND KIKRIFEYON LKKLIENKLL NKLDTYVRNC GKYNYYLQVG |
| 361 | EIATSDFIAR NRONEAFLRN IIGVSSVAYF SLRNILETEN ENDITGRMRG KTVKNNKGEE |
| 421 | KYVSGEVDKI YNENKONEVK ENLKMFYSYD FNMDNKNEIE DFFANIDEAI SSIRHGIVHF |
| 481 | NLELEGKDIF AFKNIAPSEI SKKMFQNEIN EKKLKLKIFK QLNSANVFNY YEKDVIIKYL |
| 541 | KNTKFNFVNK NIPFVPSFTK LYNKIEDLRN TLKFFWSVPK DKEEKDAQIY LLKNIYYGEF |
| 601 | LNKFVKNSKV FFKITNEVIK INKORNOKTG HYKYQKFENI EKTVPVEYLA IIQSREMINN |
| 661 | QDKEEKNTYI DFIQQIFLKG FIDYLNKNNL KYIESNNNND NNDIFSKIKI KKDNKEKYDK |
| 721 | ILKNYEKHNR NKEIPHEINE FVREIKLGKI LKYTENLNMF YLILKLLNHK ELTNLKGSLE |
| 781 | KYQSANKEET FSDELELINL LNLDNNRVTE DFELEANEIG KFLDFNENKI KDRKELKKFD |
| 841 | TNKIYFDGEN IIKHRAFYNI KKYGMLNLLE KIADKAKYKI SLKELKEYSN KKNEIEKNYT |
| 901 | MQQNLHRKYA RPKKDEKFND EDYKEYEKAI GNIQKYTHLK NKVEFNELNL LOGLLLKILH |
| 961 | RLVGYTSIWE RDLRFRLKGE FPENHYIEEI FNFDNSKNVK YKSGQIVEKY INFYKELYKD |
| 1021 | NVEKRSIYSD KKVKKLKQEK KDLYIRNYIA HFNYIPHAEI SLLEVLENLR KLLSYDRKLK |
| 1081 | NAIMKSIVDI LEKYGFVATF KIGADKKIEI QTLESEKIVH LKNLKKKKLM TDRNSEELCE |
| 1141 | LVKVMFEYKA LE |

LseCas13a, WP_012985477.1. (Liu, L. et al (2017) Cell 170 (4), 714-726)
(SEQ ID NO: 23)

| | |
|---|---|
| 1 | MWISIKTLIH HLGVLFFCDY MYNRREKKII EVKTMRITKV EVDRKKVLIS RDKNGGKLVY |
| 61 | ENEMQDNTEQ IMHHKKSSFY KSVVNKTICR PEQKQMKKLV HGLLQENSQE KIKVSDVTKL |
| 121 | NISNFLNHRF KKSLYYFPEN SPDKSEEYRI EINLSQLLED SLKKQQGTFI CWESFSKDME |
| 181 | LYINWAENYI SSKTKLIKKS IRNNRIQSTE SRSGQLMDRY MKDILNKNKP FDIQSVSEKY |
| 241 | QLEKLTSALK ATFKEAKKND KEINYKLKST LONHERQIIE ELKENSELNQ FNIEIRKHLE |
| 301 | TYFPIKKTNR KVGDIRNLEI GEIQKIVNHR LKNKIVQRIL QEGKLASYEI ESTVNSNSLQ |
| 361 | KIKIEEAFAL KFINACLFAS NNLRNMVYPV CKKDILMIGE FKNSFKEIKH KKFIRQWSQF |
| 421 | FSQEITVDDI ELASWGLRGA IAPIRNEIIH LKKHSWKKFF NNPTFKVKKS KIINGKTKDV |
| 481 | TSEFLYKETL FKDYFYSELD SVPELIINKM ESSKILDYYS SDOLNQVFTI PNFELSLLTS |
| 541 | AVPFAPSFKR VYLKGFDYQN QDEAQPDYNL KLNIYNEKAF NSEAFQAQYS LFKMVYYQVF |
| 601 | LPQFTTNNDL FKSSVDFILT LNKERKGYAK AFQDIRKMNK DEKPSEYMSY IQSQLMLYQK |
| 661 | KQEEKEKINH FEKFINQVFI KGFNSFIEKN RLTYICHPTK NTVPENDNIE IPFHTDMDDS |
| 721 | NIAFWLMCKL LDAKQLSELR NEMIKFSCSL QSTEEISTFT KAREVIGLAL LNGEKGCNDW |
| 781 | KELFDDKEAW KKNMSLYVSE ELLQSLPYTQ EDGQTPVINR SIDLVKKYGT ETILEKLFSS |
| 841 | SDDYKVSAKD IAKLHEYDVT EKIAQQESLH KQWIEKPGLA RDSAWTKKYQ NVINDISNYQ |
| 901 | WAKTKVELTO VRHLHQLTID LLSRLAGYMS IADRDFQFSS NYILERENSE YRVTSWILLS |
| 961 | ENKNKNKYND YELYNLKNAS IKVSSKNDPQ LKVDLKOLRL TLEYLELFDN RLKEKRNNIS |
| 1021 | HFNYLNGQLG NSILELFDDA RDVLSYDRKL KNAVSKSLKE ILSSHGMEVT FKPLYQTNHH |
| 1081 | LKIDKLQPKK IHHLGEKSTV SSNQVSNEYC QLVRTLLTMK |

LbmCas13a, WP_044921188.1 (hypothetical protein)
(SEQ ID NO: 24)

| | |
|---|---|
| 1 | MQISKVNHKH VAVGQKDRER ITGFIYNDPV GDEKSLEDVV AKRANDTKVL FNVFNTKDLY |
| 61 | DSQESDKSEK DKEIISKGAK FVAKSENSAI TILKKQNKIY STLTSQQVIK ELKDKFGGAR |
| 121 | IYDDDIEEAL TETLKKSFRK ENVRNSIKVL IENAAGIRSS LSKDEEELIQ EYFVKOLVEE |
| 181 | YTKTKLQKNV VKSIKNQNMV IQPDSDSQVL SLSESRREKQ SSAVSSDTLV NCKEKDVLKA |
| 241 | FLTDYAVLDE DERNSLLWKL RNLVNLYFYG SESIRDYSYT KEKSVWKEHD EQKANKTLFI |
| 301 | DEICHITKIG KNGKEQKVLD YEENRSRCRK QNINYYRSAL NYAKNNTSGI FENEDSNHFW |
| 361 | IHLIENEVER LYNGIENGEE FKFETGYISE KVWKAVINHL SIKYIALGKA VYNYAMKELS |
| 421 | SPGDIEPGKI DDSYINGITS FDYEIIKAEE SLORDISMNV VFATNYLACA TVDTDKDFLL |
| 481 | FSKEDIRSCT KKDGNLCKNI MQFWGGYSTW KNFCEEYLKD DKDALELLYS LKSMLYSMRN |
| 541 | SSFHFSTENV DNGSWDTELI GKLFEEDCNR AARIEKEKFY NNNLHMFYSS SLLEKVLERL |
| 601 | YSSHHERASQ VPSFNRVFVR KNFPSSLSEQ RITPKFTDSK DEQIWQSAVY YLCKEIYYND |
| 661 | FLQSKEAYKL FREGVKNLDK NDINNOKAAD SFKQAVVYYG KAIGNATLSQ VCQAIMTEYN |
| 721 | RONNDGLKKK SAYAEKQNSN KYKHYPLFLK QVLQSAFWEY LDENKEIYGF ISAQIHKSNV |
| 781 | EIKAEDFIAN YSSQQYKKLV DKVKKTPELQ KWYTLGRLIN PRQANQFLGS IRNYVQFVKD |
| 841 | IQRRAKENGN PIRNYYEVLE SDSIIKILEM CTKLNGTTSN DIHDYFRDED EYAEYISQFV |
| 901 | NFGDVHSGAA LNAFCNSESE GKKNGIYYDG INPIVNRNWV LCKLYGSPDL ISKIISRVNE |
| 961 | NMIHDFHKQE DLIREYQIKG ICSNKKEQQD LRTFQVLKNR VELRDIVEYS EIINELYGQL |
| 1021 | IKWCYLRERD LMYFQLGFHY LCLNNASSKE ADYIKINVDD RNISGAILYQ IAAMYINGLP |
| 1081 | VYYKKDDMYV ALKSGKKASD ELNSNEQTSK KINYFLKYGN NILGDKKDQL YLAGLELFEN |
| 1141 | VAEHENIIIF RNEIDHFHYF YDRDRSMLDL YSEVFDRFFT YDMKLRKNVV NMLYNILLDH |
| 1201 | NIVSSFVFET GEKKVGRGDS EVIKPSAKIR LRANNGVSSD VFTYKVGSKD ELKIATLPAK |
| 1261 | NEEFLLNVAR LIYYPDMEAV SEMNVREGVV KVEKSNDKKG KISRGSNTRS SNQSKYNNKS |
| 1321 | KNRMNYSMGS IFEKMDLKFD |

LbnCas13a, WP_022785443.1 (Liu, L. et al, Cell 170 (4), 714-726)
(SEQ ID NO: 25)

| | |
|---|---|
| 1 | MKISKVREEN RGAKLTVNAK TAVVSENRSQ EGILYNDPSR YGKSRKNDED RDRYIESRLK |
| 61 | SSGKLYRIFN EDKNKRETDE LOWFLSEIVK KINRRNGLVL SDMLSVDDRA FEKAFEKYAE |
| 121 | LSYTNRRNKV SGSPAFETCG VDAATAERLK GIISETNFIN RIKNNIDNKV SEDIIDRIIA |
| 181 | KYLKKSLCRE RVKRGLKKLL MNAFDLPYSD PDIDVQRDFI DYVLEDFYHV RAKSQVSRSI |
| 241 | KNMNMPVQPE GDGKFAITVS KGGTESGNKR SAEKEAFKKF LSDYASLDER VRDDMLRRMR |
| 301 | RLVVLYFYGS DDSKLSDVNE KFDVWEDHAA RRVDNREFIK LPLENKLANG KTDKDAERIR |
| 361 | KNTVKELYRN QNIGCYRQAV KAVEEDNNGR YFDDKMLNMF FIHRIEYGVE KIYANLKQVT |
| 421 | EFKARTGYLS EKIWKDLINY ISIKYIAMGK AVYNYAMDEL NASDKKEIEL GKISEEYLSG |
| 481 | ISSFDYELIK AEEMLORETA VYVAFAARHL SSQTVELDSE NSDFLLLKPK GTMDKNDKNK |
| 541 | LASNNILNFL KDKETLRDTI LQYFGGHSLW TDFPFDKYLA GGKDDVDFLT DLKDVIYSMR |
| 601 | NDSFHYATEN HNNGKWNKEL ISAMFEHETE RMTVVMKDKF YSNNLPMFYK NDDLKKLLID |
| 661 | LYKDNVERAS QVPSFNKVFV RKNFPALVRD KDNLGIELDL KADADKGENE LKFYNALYYM |
| 721 | FKEIYYNAFL NDKNVRERFI TKATKVADNY DRNKERNLKD RIKSAGSDEK KKLREQLQNY |
| 781 | IAENDFGQRI KNIVQVNPDY TLAQICQLIM TEYNQQNNGC MQKKSAARKD INKDSYQHYK |
| 841 | MLLLVNLRKA FLEFIKENYA FVLKPYKHDL CDKADFVPDF AKYVKPYAGL ISRVAGSSEL |
| 901 | QKWYIVSRFL SPAQANHMLG FLHSYKQYVW DIYRRASETG TEINHSIAED KIAGVDITDV |
| 961 | DAVIDLSVKL CGTISSEISD YFKDDEVYAE YISSYLDFEY DGGNYKDSLN RFCNSDAVND |
| 1021 | QKVALYYDGE HPKLNRNIIL SKLYGERRFL EKITDRVSRS DIVEYYKLKK ETSQYQTKGI |
| 1081 | FDSEDEQKNI KKFQEMKNIV EFRDLMDYSE IADELQGQLI NWIYLRERDL MNFQLGYHYA |
| 1141 | CLNNDSNKQA TYVTLDYQGK KNRKINGAIL YQICAMYING LPLYYVDKDS SEWTVSDGKE |
| 1201 | STGAKIGEFY RYAKSFENTS DCYASGLEIF ENISEHDNIT ELRNYIEHFR YYSSFDRSFL |
| 1261 | GIYSEVFDRF FTYDLKYRKN VPTILYNILL QHFVNVRFEF VSGKKMIGID KKDRKIAKEK |
| 1321 | ECARITIREK NGVYSEQFTY KLKNGTVYVD ARDKRYLQSI IRLLFYPEKV NMDEMIEVKE |
| 1381 | KKKPSDNNTG KGYSKRDRQQ DRKEYDKYKE KKKKEGNFLS GMGGNINWDE INAQLKN |

CamCas13a, WP_031473346.1 (hypothetical protein)
(SEQ ID NO: 26)

| | |
|---|---|
| 1 | MKFSKVDHTR SAVGIQKATD SVHGMLYTDP KKQEVNDLDK RFDQLNVKAK RLYNVFNQSK |
| 61 | AEEDDDEKRF GKVVKKLNRE LKDLLFHREV SRYNSIGNAK YNYYGIKSNP EEIVSNLGMV |
| 121 | ESLKGERDPQ KVISKLLLYY LRKGLKPGTD GLRMILEASC GLRKLSGDEK ELKVFLQTLD |
| 181 | EDFEKKTFKK NLIRSIENQN MAVQPSNEGD PIIGITQGRF NSQKNEEKSA IERMMSMYAD |
| 241 | LNEDHREDVL RKLRRLNVLY FNVDTEKTEE PTLPGEVDTN PVFEVWHDHE KGKENDRQFA |
| 301 | TFAKILTEDR ETRKKEKLAV KEALNDLKSA IRDHNIMAYR CSIKVTEQDK DGLFFEDQRI |
| 361 | NRFWIHHIES AVERILASIN PEKLYKLRIG YLGEKVWKDL LNYLSIKYIA VGKAVFHFAM |
| 421 | EDLGKTGQDI ELGKLSNSVS GGLTSFDYEQ IRADETLORQ LSVEVAFAAN NLFRAVVGQT |
| 481 | GKKIEQSKSE ENEEDFLLWK AEKIAESIKK EGEGNTLKSI LQFFGGASSW DLNHFCAAYG |
| 541 | NESSALGYET KFADDLRKAI YSLRNETFHF TTLNKGSFDW NAKLIGDMFS HEAATGIAVE |
| 601 | RTRFYSNNLP MFYRESDLKR IMDHLYNTYH PRASQVPSFN SVFVRKNFRL FLSNTLNTNT |
| 661 | SFDTEVYQKW ESGVYYLFKE IYYNSFLPSG DAHHLFFEGL RRIRKEADNL PIVGKEAKKR |
| 721 | NAVQDFGRRC DELKNLSLSA ICQMIMTEYN EQNNGNRKVK STREDKRKPD IFQHYKMLLL |
| 781 | RTLQEAFAIY IRREEFKFIF DLPKTLYVMK PVEEFLPNWK SGMFDSLVER VKQSPDLQRW |
| 841 | YVLCKFLNGR LLNOLSGVIR SYIQFAGDIQ RRAKANHNRL YMDNTQRVEY YSNVLEVVDF |
| 901 | CIKGTSRFSN VFSDYFRDED AYADYLDNYL QFKDEKIAEV SSFAALKTFC NEEEVKAGIY |
| 961 | MDGENPVMQR NIVMAKLFGP DEVLKNVVPK VTREEIEEYY QLEKQIAPYR QNGYCKSEED |
| 1021 | QKKLLRFQRI KNRVEFQTIT EFSEIINELL GOLISWSFLR ERDLLYFQLG FHYLCLHNDT |
| 1081 | EKPAEYKEIS REDGTVIRNA ILHQVAAMYV GGLPVYTLAD KKLAAFEKGE ADCKLSISKD |
| 1141 | TAGAGKKIKD FFRYSKYVLI KDRMLTDQNQ KYTIYLAGLE LFENTDEHDN ITDVRKYVDH |
| 1201 | FKYYATSDEN AMSILDLYSE IHDRFFTYDM KYQKNVANML ENILLRHFVL IRPEFFTGSK |
| 1261 | KVGEGKKITC KARAQIEIAE NGMRSEDFTY KLSDGKKNIS TCMIAARDQK YLNTVARLLY |
| 1321 | YPHEAKKSIV DTREKKNNKK TNRGDGTFNK QKGTARKEKD NGPREFNDTG FSNTPFAGFD |
| 1381 | PFRNS |

CgaCas13a, WP_034560163.1 (hypothetical protein)
(SEQ ID NO: 27)

| | |
|---|---|
| 1 | MRITKVKIKL DNKLYQVTMQ KEEKYGTLKL NEESRKSTAE ILRLKKASFN KSFHSKTINS |
| 61 | QKENKNATIK KNGDYISQIF EKLVGVDTNK NIRKPKMSLT DLKDLPKKDL ALFIKRKFKN |
| 121 | DDIVEIKNLD LISLFYNALQ KVPGEHFTDE SWADFCQEMM PYREYKNKFI ERKIILLANS |
| 181 | IEQNKGFSIN PETFSKRKRV LHQWAIEVQE RGDFSILDEK LSKLAEIYNF KKMCKRVQDE |
| 241 | LNDLEKSMKK GKNPEKEKEA YKKQKNFKIK TIWKDYPYKT HIGLIEKIKE NEELNQFNIE |
| 301 | IGKYFEHYFP IKKERCTEDE PYYLNSETIA TTVNYQLKNA LISYLMQIGK YKQFGLENQV |
| 361 | LDSKKLQEIG IYEGFQTKFM DACVFATSSL KNIIEPMRSG DILGKREFKE AIATSSFVNY |
| 421 | HHFFPYFPFE LKGMKDRESE LIPFGEQTEA KOMQNIWALR GSVQQIRNEI FHSFDKNQKF |
| 481 | NLPQLDKSNF EFDASENSTG KSQSYIETDY KFLFEAEKNQ LEQFFIERIK SSGALEYYPL |
| 541 | KSLEKLFAKK EMKFSLGSQV VAFAPSYKKL VKKGHSYQTA TEGTANYLGL SYYNRYELKE |
| 601 | ESFQAQYYLL KLIYQYVFLP NFSQGNSPAF RETVKAILRI NKDEARKKMK KNKKFLRKYA |
| 661 | FEQVREMEFK ETPDQYMSYL QSEMREEKVR KAEKNDKGFR KNITMNFEKL LMQIFVKGFD |
| 721 | VFLTTFAGKE LLLSSEEKVI KETEISLSKK INEREKTLKA SIQVEHQLVA TNSAISYWLF |
| 781 | CKLLDSRHLN ELRNEMIKFK QSRIKFNHTQ HAELIQNLLP IVELTILSND YDEKNDSQNV |
| 841 | DVSAYFEDKS LYETAPYVQT DDRTRVSFRP ILKLEKYHTK SLIEALLKDN PQFRVAATDI |
| 901 | QEWMKHREEI GELVEKRKNL HTEWAEGQQT LGAEKREEYR DYCKKIDRFN WKANKVTLTY |
| 961 | LSQLHYLITD LLGRMVGFSA LFERDLVYFS RSFSELGGET YHISDYKNLS GVLRLNAEVK |
| 1021 | PIKIKNIKVI DNEEDPYKGN EPEVKPFLDR LHAYLENVIG IKAVHGKIRN QTAHLSVLQL |
| 1081 | ELSMIESMNN LRDLMAYDRK LKNAKVTSMI KILDKHGMIL KLKIDENHKN FEIESLIPKE |
| 1141 | IIHLKDKAIK TNQVSEEYCQ LVLALLTTNP GNQLN |

Cga2Cas13a, WP_034563842.1 (hypothetical protein)
(SEQ ID NO: 28)

| | |
|---|---|
| 1 | MRMTKVKING SPVSMNRSKL NGHLVWNGTT NTVNILTKKE QSFAASFLNK TLVKADQVKG |
| 61 | YKVLAENIFI IFEQLEKSNS EKPSVYLNNI RRLKEAGLKR FFKSKYHEEI KYTSEKNQSV |
| 121 | PTKLNLIPLF FNAVDRIQED KFDEKNWSYF CKEMSPYLDY KKSYLNRKKE ILANSIQQNR |
| 181 | GFSMPTAEEP NLLSKRKQLF QQWAMKFQES PLIQQNNFAV EQFNKEFANK INELAAVYNV |
| 241 | DELCTAITEK LMNFDKDKSN KTRNFEIKKL WKQHPHNKDK ALIKLFNQEG NEALNQFNIE |
| 301 | LGKYFEHYFP KTGKKESAES YYLNPQTIIK TVGYQLRNAF VQYLLQVGKL KQYNKGVLDS |
| 361 | QTLQEIGMYE GFQTKFMDAC VFASSSLRNI IQATTNEDIL TREKFKKELE KNVELKHDLF |
| 421 | FKTEIVEERD ENPAKKIAMT PNELDLWAIR GAVQRVRNQI FHQQINKRHE PNQLKVGSFE |
| 481 | NGDLGNVSYQ KTIYQKLFDA EIKDIEIYFA EKIKSSGALE QYSMKDLEKL FSNKELTLSL |
| 541 | GGQVVAFAPS YKKLYKQGYF YQNEKTIELE QFTDYDFSND VFKANYYLIK LIYHYVFLPQ |
| 601 | FSQANNKLFK DTVHYVIQQN KELNTTEKDK KNNKKIRKYA FEQVKLMKNE SPEKYMQYLQ |
| 661 | REMQEERTIK EAKKTNEEKP NYNFEKLLIQ IFIKGFDTFL RNFDLNLNPA EELVGTVKEK |
| 721 | AEGLRKRKER IAKILNVDEQ IKTGDEEIAF WIFAKLLDAR HLSELRNEMI KFKQSSVKKG |
| 781 | LIKNGDLIEQ MQPILELCIL SNDSESMEKE SFDKIEVFLE KVELAKNEPY MQEDKLTPVK |
| 841 | FRFMKQLEKY QTRNFIENLV IENPEFKVSE KIVLNWHEEK EKIADLVDKR TKLHEEWASK |
| 901 | AREIEEYNEK IKKNKSKKLD KPAEFAKFAE YKIICEAIEN FNRLDHKVRL TYLKNLHYLM |
| 961 | IDLMGRMVGF SVLFERDFVY MGRSYSALKK QSIYLNDYDT FANIRDWEVN ENKHLFGTSS |
| 1021 | SDLTFQETAE FKNLKKPMEN QLKALLGVTN HSFEIRNNIA HLHVLRNDGK GEGVSLLSCM |
| 1081 | NDLRKLMSYD RKLKNAVTKA IIKILDKHGM ILKLTNNDHT KPFEIESLKP KKIIHLEKSN |
| 1141 | HSFPMDQVSQ EYCDLVKKML VFTN |

Pprcas13a, WP_013443710.1 (Liu, L. et al, Cell 170 (4), 714-726)
(SEQ ID NO: 29)

| | |
|---|---|
| 1 | MRVSKVKVKD GGKDKMVLVH RKTTGAQLVY SGQPVSNETS NILPEKKRQS FDLSTLNKTI |
| 61 | IKFDTAKKQK LNVDQYKIVE KIFKYPKQEL PKQIKAEEIL PFLNHKFQEP VKYWKNGKEE |
| 121 | SFNLTLLIVE AVQAQDKRKL QPYYDWKTWY IQTKSDLLKK SIENNRIDLT ENLSKRKKAL |
| 181 | LAWETEFTAS GSIDLTHYHK VYMTDVLCKM LQDVKPLTDD KGKINTNAYH RGLKKALQNH |
| 241 | QPAIFGTREV RNEANRADNQ LSIYHLEVVK YLEHYFPIKT SKRRNTADDI AHYLKAQTLK |
| 301 | TTIEKQLVNA IRANIIQQGK TNHHELKADT TSNDLIRIKT NEAFVLNLTG TCAFAANNIR |
| 361 | NMVDNEQTND ILGKGDFIKS LLKDNTNSQL YSFFFGEGLS TNKAEKETQL WGIRGAVQQI |
| 421 | NMVDNEQTND ILGKGDFIKS LLKDNTNSQL YSFFFGEGLS TNKAEKETQL WGIRGAVQQI |
| 481 | RNNVNHYKKD ALKTVFNISN FENPTITDPK QQTNYADTIY KARFINELEK IPEAFAQQLK |
| 541 | TGGAVSYYTI ENLKSLLTTF QFSLCRSTIP FAPGFKKVEN GGINYQNAKQ DESFYELMLE |
| 601 | QYLRKENFAE ESYNARYFML KLIYNNLFLP GFTTDRKAFA DSVGFVQMQN KKQAEKVNPR |
| 661 | KKEAYAFEAV RPMTAADSIA DYMAYVQSEL MQEQNKKEEK VAEETRINFE KFVLQVFIKG |
| 721 | FDSFLRAKEF DFVOMPQPQL TATASNQQKA DKLNQLEASI TADCKLTPQY AKADDATHIA |
| 781 | FYVFCKLLDA AHLSNLRNEL IKFRESVNEF KFHHLLEIIE ICLLSADVVP TDYRDLYSSE |
| 841 | ADCLARLRPF IEQGADITNW SDLFVQSDKH SPVIHANIEL SVKYGTTKLL EQIINKDTQF |
| 901 | KTTEANFTAW NTAQKSIEQL IKQREDHHEQ WVKAKNADDK EKQERKREKS NFAQKFIEKH |
| 961 | GDDYLDICDY INTYNWLDNK MHFVHLNRLH GLTIELLGRM AGFVALFDRD FQFFDEQQIA |
| 1021 | DEFKLHGFVN LHSIDKKLNE VPTKKIKEIY DIRNKIIQIN GNKINESVRA NLIQFISSKR |
| | NYYNNAFLHV SNDEIKEKOM YDIRNHIAHF NYLTKDAADF SLIDLINELR ELLHYDRKLK |

LweCas13a, WP_036059185.1 (hypothetical protein)
(SEQ ID NO: 30)

| | |
|---|---|
| 1 | MLALLHQEVP SQKLHNLKSL NTESLTKLFK PKFQNMISYP PSKGAEHVQF CLTDIAVPAI |
| 61 | RDLDEIKPDW GIFFEKLKPY TDWAESYIHY KOTTIQKSIE QNKIQSPDSP RKLVLQKYVT |
| 121 | AFLNGEPLGL DLVAKKYKLA DLAESFKVVD LNEDKSANYK IKACLQQHQR NILDELKEDP |
| 181 | ELNQYGIEVK KYIQRYFPIK RAPNRSKHAR ADFLKKELIE STVEQQFKNA VYHYVLEQGK |
| 241 | MEAYELTDPK TKDLQDIRSG EAFSFKFINA CAFASNNLKM ILNPECEKDI LGKGDFKKNL |
| 301 | PNSTTQSDVV KKMIPFFSDE IQNVNFDEAI WAIRGSIQQI RNEVYHCKKH SWKSILKIKG |
| 361 | FEFEPNNMKY TDSDMQKLMD KDIAKIPDFI EEKLKSSGII RFYSHDKLQS IWEMKQGFSL |
| 421 | LTTNAPFVPS FKRVYAKGHD YQTSKNRYYD LGLTTFDILE YGEEDFRARY FLTKLVYYQQ |
| 481 | FMPWFTADNN AFRDAANFVL RLNKNRQQDA KAFINIREVE EGEMPRDYMG YVQGQIAIHE |
| 541 | DSTEDTPNHF EKFISQVFIK GFDSHMRSAD LKFIKNPRNQ GLEQSEIEEM SFDIKVEPSF |
| 601 | LKNKDDYIAF WTFCKMLDAR HLSELRNEMI KYDGHLTGEQ EIIGLALLGV DSRENDWKQF |
| 661 | FSSEREYEKI MKGYVGEELY QREPYRQSDG KTPILFRGVE QARKYGTETV IQRLFDASPE |
| 721 | FKVSKCNITE WERQKETIEE TIERRKELHN EWEKNPKKPQ NNAFFKEYKE CCDAIDAYNW |
| 781 | HKNKTTLVYV NELHHLLIEI LGRYVGYVAI ADRDFQCMAN QYFKHSGITE RVEYWGDNRL |
| 841 | KSIKKLDTFL KKEGLFVSEK NARNHIAHLN YLSLKSECTL LYLSERLREI FKYDRKLKNA |
| 901 | VSKSLIDILD RHGMSVVFAN LKENKHRLVI KSLEPKKLRH LGEKKIDNGY IETNQVSEEY |
| 961 | CGIVKRLLEI |

LneCas13a, WP_036091002.1 (hypothetical protein)
(SEQ ID NO: 31)

| | |
|---|---|
| 1 | MKITKMRVDG RTIVMERTSK EGQLGYEGID GNKTTEIIFD KKKESFYKSI KNKTVRKPDE |
| 61 | KEKNRRKQAI NKAINKEITE LMLAVLHQEV PSQKLHNLKS LNTESLTKLF KPKFQNMISY |
| 121 | PPSKGAEHVQ FCLTDIAVPA IRDLDEIKPD WGIFFEKLKP YTDWAESYIH YKQTTIQKSI |
| 181 | EQNKIQSPDS PRLLVLQKYV TAFLNGEPLG LDLVAKKYKL ADLAESFKLV DLNEDKSANY |
| 241 | KIKACLQQHQ RNILDELKED PELNQYGIEV KKYIQRYFPI KRAPNRSKHA RADFLKKELI |
| 301 | ESTVEQQFKN AVYHYVLEQG KMEAYELTDP KTKDLQDIRS GEAFSFKFIN ACAFASNNLK |
| 361 | MILNPECEKD ILGKGNFKKN LPNSTTRSDV VKKMIPFFSD ELQNVNFDEA IWAIRGSIQQ |
| 421 | IRNEVYHCKK HSWKSILKIK GFEFEPNNMK YADSDMQKLM DKDIAKIPEF IEEKLKSSGV |
| 481 | VRFYRHDELQ SIWEMKQGFS LLTTNAPFVP SFKRVYAKGH DYQTSKNRYY NLDLTTFDIL |
| 541 | YEGEEDFRAT YFLTKLVYYQ QFMPWFTADN NAFRDAANFV LRLNKNRQQD AKAFINIREV |
| 601 | EEGEMPRDYM GYVQGQIAIH EDSIEDTPNH FEKFISQVFI KGFDRHMRSA NLKFTINPRN |
| 661 | QGLEQSEIEE MSFDIKVEPS FLKNKDDYIA FWIFCKMLDA RHLSELRNEM IKYDGHLTGE |
| 721 | QEIIGLALLG VDSRENDWKQ FFSSEREYEK IMKGYVVEEL YQREPYRQSD GKTPILFRGV |
| 781 | EQARKYGTET VIQRLFDANP EFKVSKCNLA EWERQKETIE ETIKRRKELH NEWAKNPKKP |
| 841 | QNNAFFKEYK ECCDAIDAYN WHKNKTTLAY VNELHHLLIE ILGRYVGYVA IADRDFQCMA |
| 901 | NQYFKHSGIT ERVEYWGDNR LKSIKKLDTF LKKEGLFVSE KNARNHIAHL NYLSLKSECT |
| 961 | LLYLSERLRE IFKYDRKLKN AVSKSLIDIL DRHGMSVVFA NLKENKHRLV IKSLEPLLKR |
| 1021 | HLGGKKIDGG YIETNQVSEE YCGIVKRLLE M |

Lwa2cas13a, WP_021746774.1 (hypothetical protein)
(SEQ ID NO: 32)

| | |
|---|---|
| 1 | MKVTKVDGIS HKKYIEEGKL VKSTSEENRT SERLSELLSI RLDIYIKNPD NASEEENRIR |
| 61 | RENLKKFFSN KVLHLKDSVL YLKNRKEKNA VQDKNYSEED ISEYDLKNKN SFSVLKKILL |
| 121 | NEDVNSEELE IFRKDVEAKL NKINSLKYSF EENKANYQKI NENNVEKVGG KSKRNIIYDY |
| 181 | YRESAKRNDY INNVQEAFDK LYKKEDIEKL FFLIENSKKH EKYKIREYYH KIIGRKNDKE |
| 241 | NFAKIIYEEI QNVNNIKELI EKIPDMSELK KSQVFYKYYL DKEELNDKNI KYAFCHFVEI |
| 301 | EMSQLLKNYV YKRLSNISND KIKRIFEYQN LKKLIENKLL NKLDTYVRNC GKYNYYLQVG |
| 361 | EIATSDFIAR NRQFEAFLRN IIGVSSVAYF SLRNILETEN ENDITGRMRG KTVKNNKGEE |
| 421 | KYVSGEVDKI YNENKQNEVK ENLKMFYSYD FNMDNKNEIE DFFANIDEAI SSIRHGIVHF |
| 481 | NLELEGKDIF AFKNIAPSEI SKKMFQNEIN EKKLKLKIFK QLNSANVFNY YEKDVIIKYL |
| 541 | KNTKFNFVNK NIPFVPSFTK LYNKIEDLRN TLKFFWSVPK DKEEKQAQIY LLKNIYYGEF |
| 601 | LNKFVKNSKV FFKITNEVIK INKQRNQKTG HYKYQKFENI EKTVPVEYLA IIQSREMINN |
| 661 | QDKEEKNTYI DFIQQIFLKG FIDYLNKNNL KYIESNNNND NNDIFSKIKI KKDNKEKYDK |
| 721 | ILKNYEKHNR NKEIPHEINE FVREIKLGKI LKYTENLNMF YLILKLLNHK ELTNLKGSLE |
| 781 | KYQSANKEET FSDELELINL LNLDNNRVTE DFELEANEIG KFLDFNENKI KDRKELKKFD |
| 841 | TNKIYFDGEN IIKHRAFYNI KKYGMLNLLE KIADKAKYKI SLKELKEYSN KKNEIEKNYT |
| 901 | MQQNLHRKYA RPKKDEKFND EDYKEYEKAI GNIQKYTHLK NKVEFNELNL LQGLLLKILH |
| 961 | RLVGYTSIWE RDLRFRLKGE FPENHYIEEI FNFDNSKNVK YKSGQIVEKY INFYKELYKD |
| 1021 | NVEKRSIYSD KKVKKLKQEK KDLYIRNYIA HFNYIPHAEI SLLEVLENLR KLLSYDRKLK |
| 1081 | NAIMKSIVDI LKEYGFVATF KIGADKKIEI QTLELEKIVH LKNLKKKKLM TDRNSEELCE |
| 1141 | LVKVMFEYKA LE |

RcsCas13a, WP_013067728.1 (hypothetical protein)
(SEQ ID NO: 33)

| | |
|---|---|
| 1 | MQIGKVQGRT ISEFGDPAGG LKRKISTDGK NRKELPAHLS SDPKALIGQW ISGIDKIYRK |
| 61 | PDSRKSDGKA IHSPTPSKMQ FDARDDLGEA NWKLVSEAGL AQDSDYDQFK RRLHPYGDKF |
| 121 | QPADSGAKLK FEADPPEPQA FHGRWYGAMS KRGNDAKELA AALYEHLHVD EKRIDGQPKR |
| 181 | NPKTDKFAPG LVVARALGIE SSVLPRGMAR LARNWGEEEI QTYFVVDVAA SVKEVAKAAV |
| 241 | SAAQAFDPPR QVSGRSLSPK VGFALAEHLE RVTGSKRCSF DPAAGPSVLA LHDEVKKTYK |
| 301 | RLCARGKNAA RAFPADKTEL LALMRHTHEN RVRNQMVRMG RVSEYRGQQA GDLAQSHYWT |
| 361 | SAGQTEIKES EIFVRLWVGA FALAGRSMKA WIDPMGKIVN TEKNDRDLTA AVNIRQVISN |
| 421 | KEMVAEAMAR RGIYFGETPE LDRLGAEGNE GFVFALLRYL RGCRNQTFHL GARAGFLKEI |
| 481 | RKELEKTRWG KAKEAEHVVL TDKTVAAIRA IIDNDAKALG ARLLADLSGA FVAHYASKEH |
| 541 | FSTLYSEIVK AVKDAPEVSS GLPRLKLLLK RADGVRGYVH GLRDTRKHAF ATKLPPPPAP |
| 601 | RELDDPATKA RYIALLYLYD GPFRAYASGI TGTALAGPAA RAKEAATALA QSVNVTKAYS |
| 661 | DVMEGRTSRL RPPNDGETLR EYLSALTGET ATEFRVQIGY ESDSENARKQ AEFIENYRRD |
| 721 | MLAFMFEDYI RAKGFDWILK IEPGATAMTR APVLPEPIDT RGQYEHWQAA LYLVMHFVPA |
| 781 | SDVSNLLHQL RKWEALQGKY ELVQDGDATD QADARREALD LVKRFRDVLV LFLKTGEARF |
| 841 | EGRAAPFDLK PFRALFANPA TFDRLFMATP TTARPAEDDP EGDGASEPEL RVARTLRGLR |
| 901 | QIARYNHMAV LSDLFAKHKV RDEEVARLAE IEDETQEKSQ IVAAQELRTD LHDKVMKCHP |
| 961 | KTISPEERQS YAAAIKTIEE HRFLVGRVYL GDHLRLHRLM MDVIGRLIDY AGAYERDTGT |
| 1021 | FLINASKQLG AGADWAVTIA GAANTDARTQ TRKDLAHFNV LDRADGTPDL TALVNRAREM |
| 1081 | MAYDRKRKNA VPRSILDMLA RLGLTLKWQM KDHLLQDATI TQAAIKHLDK VRLTVGGPAA |
| 1141 | VTEARDSQDY LQMVAAVFNG SVQNPKPRRR DDGDAWHKPP KPATAQSQPD QKPPNKAPSA |
| 1201 | GSRLPPPQVG EVYEGVVVKV IDTGSLGFLA VEGVAGNIGL HISRLRRIRE DAIIVGRRYR |
| 1261 | FRVEIYVPPK SNTSKLNAAD LVRID |

RcrCas13a, WP_023911507.1 (hypothetical protein)
(SEQ ID NO: 34)

| | |
|---|---|
| 1 | MQIGKVQGRT ISEFGDPAGG LKRKISTDGK NRKELPAHLS SDPKALIGQW ISGIDKIYRK |
| 61 | PDSRKSDGKA IHSPTPSKMQ FDARDDLGEA FWKLVSEAGL AQSDSYDQFK RRLHPYGDKF |
| 121 | QPADSGAKLK FEADPPEPQA FHGRWYGAMS KRGNDAKELA AALYEHLHVD EKRIDGQPKR |
| 181 | NPKTDKFAPG LVVARALGIE SSVLPRGMAR LARNWGEEEI QTYFVVDVAA SVKEVAKAAV |
| 241 | SAAQAFDPPR QVSGRSLSPK VGFALAEHLE RVTGSKRCSF DPAAGPSVLA LHDEVKKTYK |
| 301 | RLCARGKNAA RAFPADKTEL ALAMRHTHEN RVRNQMVRMG RVSEYRGQQA GDLAQSHYWT |
| 361 | SAGQTEIKES EIFVRLWVGA FALAGRSMKA WIDPMGKIVN TEKNDRDLTA AVNIRQVISN |
| 421 | KEMVAEAMAR RGIYFGETPE LDRLGAEGEN GFVFALLRYL RGCRNQTFHL GARAGFLKEI |
| 481 | RKELEKTRWG KAKEAEHVVL TDKTVAAIRA IIDNDAKALG ARLLADLSGA FVAHYASKEH |
| 541 | FSTLYSEIVK AVKDAPEVSS GLPRLKLLLK RADGVRGYVH GLRDTRKHAF ATKLPPPPAP |
| 601 | RELDDPATKA RYIALLRLYD GPFRAYASGI TGTALAGPAA RAKEAATALA QSVNVTKAYS |
| 661 | DVMEGRSSRL RPPNDGETLR EYLSALTGET ATEFRVQIGY ESDSENARKQ AEFIENYRRD |
| 721 | MLAFMFEDYI RAKGFDWILK IEPGATAMTR APVLPEPIDT RGQYEHWQAA LYLVMHFVPA |
| 781 | SDVSNLLHQL RKWEALQGKY ELVQDGDATD QADARREALD LVKRFRDVLV LFLKTGEARF |
| 841 | EGRAAPFDLK PFRALFANPA TFDRLFMATP TTARPAEDDP EGDGASEPEL RVARTLRGLR |
| 901 | QIARYNHMAV LSDLFAKHKV RDEEVARLAE IEDETQEKSQ IVAAQELRTD LHDKVMKCHP |
| 961 | KTISPEERQS YAAAIKTIEE HRFLVGRVYL GDHLRLHRLM MDVIGRLIDY AGAYERDTGT |
| 1021 | FLINASKQLG AGADWAVTIA GAANTDARTQ TRKDLAHFNV LDRADGTPDL TALVNRAREM |
| 1081 | MAYDRKRKNA VPRSILDMLA RLGLTLKWQM KDHLLQDATA TQAAIKHLDK VRLTVGGPAA |
| 1141 | VTEARFSQDY LQMVAAVFNG SVQNPKPRRR DDGDAWHKPP KPATAQSQPD QKPPNKAPSA |
| 1201 | GSRLPPPQVG EVYEGVVVKV IDTGSLGFLA VEGVAGNIGL HISRLRRIRE DAIIVGRRYK |
| 1261 | FRVEIYVPPK SNTSKLNAAD LVRID |

RcdCas13a, WP_023911507.1 (hypothetical protein)
(SEQ ID NO: 35)

| | |
|---|---|
| 1 | MQIGKVQGRT ISEFGDPAGG LKRKISTDGK NRKELPAHLS SDPKALIGQW ISGIDKIYRK |
| 61 | PDSRKSDGKA IHSPTPSKMQ FDARDDLGEA FWKLVSEAGL AQDSDYDQFK RRLHPYGDKF |
| 121 | QPADSGAKLK FEADPPEPQA FHGRWYGAMS KRGNDAKELA AALYEHLHVD EKIRDGQPKR |
| 181 | NPKTDKFAPG LVVARALGIE SSVLPRGMAR LARNWGEEEI QTYFVVDVAA SVKEVAKAAV |
| 241 | SAAQAFDPPR QVSGRSLSPK VGFALAEHLE RVTGSKRCSF DPAAGPSVLA LHDEVKKTYK |
| 301 | RLCARGKNAA RAFPADKTEL ALAMRHTHEN RVRNQMVRMG RVSEYRGQQA GDLAQSHYWT |
| 361 | SAGQTEIKES EIFVRLWVGA FALAGRSMKA WIDPMGKIVN TEKNDRDLTA AVNIRQVISN |
| 421 | KEMVAEAMAR RGIYFGETPE LDRLGAEGNE GFVFALLRYL RGCRNQTFHL GARAGFLEKI |
| 481 | RKELEKTRWG KAKEAEHVVL TDKTVAAIRA IIDNDAKALG ARLLADLSGA FVAHYASKEH |
| 541 | FSTLYSEIVK AVKDAPEVSS GLPRLKLLLK RADGVRGYVH GLRDTRKHAF ATKLPPPPAP |
| 601 | RELDDPATKA RYIALLRLYD GPFRAYASGI TGTALAGPAA RAKEAATALA QSVNVTKASY |
| 661 | DVMEGRSSRL RPPNDGETLR EYLSALTGET ATEFRVQIGY ESDSENARKQ AEFIENYRRD |
| 721 | MLAFMFEDYI RAKGFDWILK IEPGATAMTR APVLPEPIDT RGQYEHWQAA LYLVMHFVPA |
| 781 | SDVSNLLHQL RKWEALQGKY ELVQDGDATD QADARREALD LVKRFRDVLV LFLKTGEARF |
| 841 | EGRAAPFDLK PFRALFANPA TFDRLFMATP TTARPAEDDP EGDGASEPEL RVARTLRGLR |
| 901 | QIARYNHMAV LSDLFAKHKV RDEEVARLAE IEDETQEKSQ IVAAQELRTD LHDKVMKCHP |
| 961 | KTISPEERQS YAAAIKTIEE HRFLVGRVYL GDHLRLHRLM MDVIGRLIDY AGAYERDTGT |
| 1021 | FLINASKQLG AGADWAVTIA GAANTDARTQ TRKDLAHFNV LDRADGTPDL TALVNRAREM |
| 1081 | MAYDRKRKNA VPRSILDMLA RLGLTLKWQM KDHLLQDATI TQAAIKHLDK VRLTVGGPAA |
| 1141 | VTEARFSQDY LQMVAAVFNG SVQNPKPRRR DDGDAWHKPP KPATAQSQPD QKPPNKAPSA |
| 1201 | GSRLPPPQVG EVYEGVVVKV IDTGSLGFLA VEGVAGNIGL HISRLRRIRE DAIIVGRRYR |
| 1261 | FRVEIYVPPK SNTSKLNAAD LVRID |

LbuCas13a, WP_015770004, (Liu, L. et al, Cell 170 (4), 714-726)
(SEQ ID NO: 36)

| | |
|---|---|
| 1 | MKVTKVGGIS HKKYTSEGRL VKSESEENRT DERLSALLNM RLDMYIKNPS STETKENQKR |
| 61 | IGKLKKFFSN KMVYLKDNTL SLKNGKKENI DREYSETDIL ESDVRDKKNV AVLKKKYLNE |
| 121 | NVNSEELEVF RNDIKKKLNK INSLKYSFEK NKANYQKINE NNIEKVEGKS KRNIIYDYYR |
| 181 | ESAKRDAYVS NVKEAFDKLY KEEDIAKLVL EIENLTKLEK YKIREFYHEI IGRKNDKENF |
| 241 | AKIIYEEIQN VNNMKELIEK VPDMSELKKS QVFYKYYLDK EELNDKNIKY AFCHFVEIEM |
| 301 | SQLLKNYVYK RLSNISNDKI KRIFEYQNLK KLIENKLLNK LDTYVRNCGK YNYYLQDGEI |
| 361 | ATSDFIARNR QNEAFLRNII GVSSVAYFSL RNILETENEN DITGRMRGKT VKNNKGEEKY |
| 421 | VSGEVDKIYN ENKKNEVKEN LKMFYSYDFN MDNKNEIEDF FANIDEAISS IRHGIVHFNL |
| 481 | ELEGKDIFAF KNIAPSEISK KMFQNEINEK KLKLKIFRQL NSANVFRYLE KYKILNYLKR |
| 541 | TRFEFVNKNI PFVPSFTKLY SRIDDLKNSL GIYWKTPKTN DDNKTKEIID AQIYLLKNIY |
| 601 | YGEFLNYFMS NNGNFFEISK EIIELNKNDK RNLKTGFYKL QKFEDIQEKI PKEYLANIQS |
| 661 | LYMINAGNQD EEEKDTYIDF IQKIFLKGFM TYLANNGRLS LIYIGSDEET NTSLAEKKQE |
| 721 | FDKFLKKYEQ NNNINIPYEI NEFLREIKLG NILKYTERLN MFYLILKLLN HKELTNLKGS |
| 781 | LEKYQSANKE EAFSDQLELI NLLNLDNNRV TEDFELEADE IGKFLDFNGN KVKDNKELKK |
| 841 | FDTNKIYFDG ENIIKHRAFY NIKKYGMLNL LEKIADKAGY KISIEELKKY SNKKNEIEKN |
| 901 | HKMQNLLHRK YARPRKDEFK TDEDYESYKQ AIENIEEYTH LKNKVEFNEL NLLQGLLLRI |
| 961 | LHRLVGYTSI WERDLRFRLK GEFPENQYIE EIFNFENKKN VKYKGGQIVE KYIKFYKELH |
| 1021 | QNDEVKINKY SSANIKVLKQ EKKDLYIRNY IAHFNYIPHA EISLLEVLEN LRKLLSYDRK |
| 1081 | LKNAVMKSVV DILKEYGFVA TFKIGADKKI GIQTLESEKI VHLKNLKKKK LMTDRNSEEL |
| 1141 | CKLVKIMFEY KMEEKKSEN |

RcaCas13a, ETD76934.1 (Ding, H. et al (2014) Genome Announc 2 (1),
e00050-14)
(SEQ ID NO: 37)

| | |
|---|---|
| 1 | MQIGKVQGRT ISEFGDPAGG LKRKISTDGK NRKELPAHLS SDPKALIGQW ISGIDKIYRK |
| 61 | PDSRKSDGKA IHSPTPSKMQ FDARDDLGEA FWKLVSEAGL AQDSDYDQFK RRLHPYGDKF |
| 121 | QPADSGAKLK FEADPPEPQA FHGRWYGAMS KRGNDAKELA AALYEHLHVD EKRIDGQPKR |
| 181 | NPKTDKFAPG LVVARALGIE SSVLPRGMAR LARNWGEEEI QTYFVVDVAA SVKEVAKAAV |
| 241 | SAAQAFDPPR QVSGRSLSPK VGFALAEHLE RVTGSKRCSF DPAAGPSVLA LHDEVKKTYK |
| 301 | RLCARGKNAA RAFPADKTEL LALMRHTHEN RVRNQMVRMG RVSEYRGQQA GDLAQSHYWT |
| 361 | SAGQTEIKES EIFVRLWVGA FALAGRSMKA WIDPMGKIVN TEKNDRDLTA AVNIRQVISN |
| 421 | KEMVAEAMAR RGIYFGETPE LDRLGAEGNE GFVFALLRYL RGCRNQTFHL GARAGFLKEI |
| 481 | RKELEKTRWG KAKEAEHVVL TDKTVAAIRA IIDNDAKALG ARLLADLSGA FVAHYASKEH |
| 541 | FSTLYSEIVK AVKDAPEVSS GLPRLKLLLK RADGVRGYVH GLRDTRKHAF ATKLPPPPAP |
| 601 | RELDDPATKA RYIALLRLYD GPFRAYASGI TGTALAGPAA RAKEAATALA QSVNVTKAYS |
| 661 | DVMEGRSSRL RPPNDGETLR EYLSALTGET ATEFRVQIGY ESDSENARKQ AEFIENYRRD |
| 721 | MLAFMFEDYI RAKGFDWILK IEPGATAMTR APVLPEPIDT RGQYEHWQAA LYLVMHFVPA |
| 781 | SDVSNLLHQL RKWEALQGKY ELVQDGDATD QADARREALD LVKRFRDVLV LFLKTGEARF |
| 841 | EGRAAPFDLK PFRALFANPA TFDRLFMATP TTARPAEDDP EGDGASEPEL RVARTLRGLR |
| 901 | QIARYNHMAV LSDLFAKHKV RDEEVARLAE IEDETQEKSQ IVAAQELRTD LHDKVMKCHP |
| 961 | KTISPEERQS YAAAIKTIEE HRFLVGRVYL GDHLRLHRLM MDVIGRLIDY AGAYERDTGT |
| 1021 | FLINASKQLG AGADWAVTIA GAANTDARTQ TRKDLAHFNV LDRADGTPDL TALVNRAREM |
| 1081 | MAYDRKRKNA VPRSILDMLA RLGLTLKWQM KDHLLQDATI TQAAIKHLDK VRLTVGGPAA |
| 1141 | VTEARFSQDY LQMVAAVFNG SVQNPKPRRR DDGDAWHKPP KPATAQSQPD QKPPNKAPSA |
| 1201 | GSRLPPPQVG EVYEGVVVKV IDTGSLGFLA VEGVAGNIGL HISRLRRIRE DAIIVGRRYR |
| 1261 | FRVEIYVPPK SNTSKLNAAD LVRID |

EreCas13a, WP_055061018.1 (hypothetical protein)
(SEQ ID NO: 38)

| | |
|---|---|
| 1 | MLRRDKEVKK LYNVFNQIQV GTKPKKWNND EKLSPEENER RAQQKNIKMK NYKWREACSK |
| 61 | YVESSQRIIN DVIFYSYRKA KNKLRYMRKN EDILKKMQEA EKLSKFSGGK LEDFVAYTLR |
| 121 | KSLVVSKYDT QEFDSLAAMV VFLECIGKNN ISDHEREIVC KLLELIRKDF SKLDPNVKGS |
| 181 | QGANIVRSVR NQNMIVQPQG DRFLFPQVYA KENETVTNKN VEKEGLNEFL LNYANLDDEK |
| 241 | RAESLRKLRR ILDVYFSAPN HYEKDMDITL SDNIEKEKFN VWEKHECGKK ETGLFVDIPD |
| 301 | VLMEAEAENI KLDAVVEKRE RKVLNDRVRK QNIICYRYTR AVVEKYNSNE PLFFENNAIN |
| 361 | QYWIHHIENA VERILKNCKA GKLFKLRKGY LAEKVWKDAI NLISIKYIAL GKAVYNFALD |
| 421 | DIWKDKKNKE LGIVDERIRN GITSFDYEMI KAHENLQREL AVDIAFSVNN LARAVCDMSN |
| 481 | LGNKESDFLL WKRNDIADKL KNKDDMASVS AVLQFFGGKS SWDINIFKDA YKGKKKYNYE |
| 541 | VRFIDDLRKA IYCARNENFH FKTALVNDEK WNTELFGKIF ERETEFCLNV EKDRFYSNNL |
| 601 | YMFYQVSELR NMLDHLYSRS VSRAAQVPSY NSVIVRTAFP EYITNVLGYQ KPSYDADTLG |
| 661 | KWYSACYYLL KEIYYNSFLQ SDRALQLFEK SVKTLSWDDK KQQRAVDNFK DHFSKIKSAC |
| 721 | TSLAQVCQIY MTEYNQQNNQ IKKVRSSNDS IFDQPVYQHY KVLLKKAIAN AFADYLKNNK |
| 781 | DLFGFIGKPF KANEIREIDK EQFLPDWTSR KYEALCIEVS GSQELQKWYI VGKFLNARSL |
| 841 | NLMVGSMRSY IQYVTDIKRR AASIGNELHN SVHDVEKVEK WVQVIEVCSL LARRTSNQFE |
| 901 | DYFNDKDDYA RYLKSYVDFS NVDMPSEYSA LVDFSNEEQS DLYVDPKNPK VNRNIVHSKL |
| 961 | FAADHILRDI VEPVSKDNIE EFYSQKAEIA YCKIKGKEIT AEEQKAVLKY QKLKNRVELR |
| 1021 | DIVEYGEIIN ELLGQLINWS FMRERDLLYF QLGFHYDCLR NDSKKPEGYK NIKVDENSIK |
| 1081 | DAILYQIIGM YVNGVTVYAP EKDGDKLKEQ CVKGGVGVKV SAFHRYSKYL GLNEKTLYNA |
| 1141 | GLEIFEVVAE HEDIINLRNG IDHFKYYLGD YRSMLSIYSE VFDRFFTYDI KYQKNVLNLL |
| 1201 | QNILLRHNVI VEPILESGFK TIGEQTKPGA KLSIRSIKSD TFQYKVKGGT LITDAKDERY |
| 1261 | LETIRKILYY AENEEDNLKK SVVVTNADKY EKNKESDDQN KQEKEENKDN KGKKNEETKS |
| 1321 | DAEKNNNERL SYNPFANLNF KLSN |

HheCas13a, CRZ35554.1 (Wibberg, Daniel, direct submission)
(SEQ ID NO: 39)

| | |
|---|---|
| 1 | MLLTRRRISG NSVDQKITAA FYRDMSQGLL YYDSEDNDCT DKVIESMDFE RSWRGRILKN |
| 61 | GEDDKNPFYM FVKGLVGSND KIVCEPIDVD SDPDNLDILI NKNLTGFGRN LKAPDSNDTL |
| 121 | ENLIRKIQAG IPEEEVLPEL KKIKEMIQKD IVNRGEQLLK SIKNNIRPFS LEGSKLVPST |
| 181 | KKMKWLFKLI DVPNKTFNEK MLEKYWEIYD YDKLKANITN RLDKTDKKAR SISRAVSEEL |
| 241 | REYHKNLRTN YNRFVSGDRP AAGLDNGGSA KYNPDKEEFL LFLKEVEQYF KKYFPVKSKH |
| 301 | SNKSKDKSLV DKYKNYCSYK VVKKEVNRSI INQLVAGLIQ QGKLLYYFYY NDTWQEDFLN |
| 361 | SYGLSYIQVE EAFKKSVMTS LSWGINRLTS FFIDDSNTVK FDDITTKKAK EAIESNYFNK |
| 421 | LRTCSRMQDH FKEKLAFFYP VYVKDKKDRP DDDIENLIVL VKNAIESVSY LRNRTFHFKE |
| 481 | SSLLELLKEL DDKNSGQNKI DYSVAAEFIK RDIENLYDVF REQIRSLGIA EYYKADMISD |
| 541 | CFKTCGLEFA LYSPKNSLMP AFKNVYKRGA NLNKAYIRDK GPKETGDQGQ NSYKALEEYR |
| 601 | ELTWYIEVKN NDQSYNAYKN LLQLIYYHAF LPEVRENEAL ITDFINRTKE WNRKETEERL |
| 661 | NTKNNKKHKN FDENDDITVN TYRYESIPDY QGESLDDYLK VLQRKQMARA KEVNEKEEGN |
| 721 | NNYIQFIRDV VVWAFGAYLE NKLKNYKNEL QPPLSKENIG LNDTLKELFP EEKVKSPFNI |
| 781 | KCRFSISTFI DNKGKSTDNT SAEAVKTDGK EDEKDKKNIK RKDLLCFYLF LRLLDENEIC |
| 841 | KLQHQFIKYR CSLKERRFPG NRTKLEKETE LLAELEELME LVRFTMPSIP EISAKAESGY |
| 901 | DTMIKKYFKD FIEKKVFKNP KTSNLYYHSD SKTPVTRKYM ALLMRSAPLH LYKDIFKGYY |
| 961 | LITKKECLEY IKLSNIIKDY QNSLNELHEQ LERIKLKSEK QNGKDSLYLD KKDFYKVKEY |
| 1021 | VENLEQVARY KHLQHKINFE SLYRIFRIHV DIAARMVGYT QDWERDMHFL FKALVYNGVL |
| 1081 | EERRFEAIFN NNDDNNDGRI VKKIQNNLNN KNRELVSMLC WNKKLNKNEF GAIIWKRNPI |
| 1141 | AHLNHFTQTE QNSKSSLESL INSLRILLAY DRKRQNAVTK TINDLLLNDY HIRIKWEGRV |
| 1201 | DEGQIYFNIK EKEDIENEPI IHLKHLHKKD CYIYKNSYMF DKQKEWICNG IKEEVYDKSI |
| 1261 | LKCIGNLFKF DYEDKNKSSA NPKHT |

LbaCas13a, WP_022785443.1 ((Lie, L. et al, Cell 170 (4), 714-726)
(SEQ ID NO: 44)

| | |
|---|---|
| 1 | MKISKVREEN RGAKLTVNAK TAVVSENRSQ EGILYNDPSR YGKSRKNDED RDRYIESRLK |
| 61 | SSGKLYRIFN EDKNKRETDE LQWFLSEIVK KINRRNGLVL SDMLSVDDRA FEKAFEKYAE |
| 121 | LSYTNRRNKV SGSPAFETCG VDAATAERLK GIISETNFIN RIKNNIDNKV SEDIIDRIIA |
| 181 | KYLKKSLCRE RVKRGLKKLL MNAFDLPYSD PDIDVQRDFI DYVLEDFYHV RAKSQVSRSI |
| 241 | KNMNMPVQPE GDGKFAITVS KGGTESGNKR SAEKEAFKKF LSDYASLDER VRDDMLRRMR |
| 301 | RLVVLYFYGS DDSKLSDVNE KFDVWEDHAA RRVDNREFIK LPLENKLANG KTDKDAERIR |
| 361 | KNTVKELYRN QNIGCYRQAV KAVEEDNNGR YFDDKMLNMF FIHRIEYGVE KIYANLKQVT |
| 421 | EFKARTGYLS EKIWKDLINY ISIKYIAMGK AVYNYAMDEL NASDKKEIEL GKISEEYLSG |
| 481 | ISSFDYELIK AEEMLQRETA VYVAFAARHL SSQTVELDSE NSDFLLLKPK GTMDKNDKNK |
| 541 | LASNNILNFL KDKETLRDTI LQYFGGHSLW TDFPFDKYLA GGDKKVDFLT DLKDVIYSMR |
| 601 | NDSFHYATEN HNNGKWNEKL ISAMFEHETE RMTVVMKDKF YSNNLPMFYK NKKLKKLLID |
| 661 | LYKDNVERAS QVPSFNKVFV RKNFPALVRD KDNLGIELDL KADADKGENE LKFYNALYYM |
| 721 | FKEIYYNAFL NKDNVRERFI TKATKVADNY DRNKERNLKD RIKSAGSDEK KKLREQLQNY |
| 781 | IAENDFGQRI KNIVQVNPDY TLAQICQLIM TEYNQQNNGC MQKKSAARKD INKDSYQHYK |
| 841 | MLLLVNLRKA FLEFIKENYA FVLKPYKHDL CDKADFVPDF AKYVKPYAGL AIRVAGSSEL |
| 901 | QKWYIVSRFL SPAQANHMLG FLHSYKQYVW DIYRRASETG TEINHSIAED KAIGVDITDV |
| 961 | DAVIDLSVKL CGTISSEISD YFKDDEVYAE YISSYLDFEY DGGNYKDSLN RFCNSDAVND |
| 1021 | QKVALYYDGE HPKLNRNIIL SKLYGERRFL EKITDRVSRS DIVEYYKLKK ETSQYQTKGI |
| 1081 | FDSEDEQKNI KKFQEMKNIV EFRDLMDYSE IADELQGQLI NWIYLRERDL MNFQLGYHYA |
| 1141 | CLNNDSNKQA TYVTLDYQGK KNRKINGAIL YQICAMYING LPLYYVDKDS SEWTVSDGKE |
| 1201 | STGAKIGEFY RYAKSFENTS DCYASGLEIF ENISEHDNIT ELRNYIEHFR YYSSFDRSFL |
| 1261 | GIYSEVFDRF FTYDLKYRKN VPTILYNILL QHFVNVRFEF VSGKK |
| 1321 | |
| 1381 | |

Cas13d
[Eubacterium] siraeum DSM 15702 ESCas13d
(SEQ ID NO: 40)

| | |
|---|---|
| 1 | MGKKIHARDL REQRKTDRTE KFADQNKKRE AERAVPKKDA AVSVKSVSSV SSKKDNVTKS |
| 61 | MAKAAGVKSV FAVGNTVYMT SFGRGNDAVL EQKIVDTSHE PLNIDDPAYQ LNVVTMNGYS |
| 121 | VTGHRGETVS AVTDNPLRRF NGRKKDPEEQ SVPTDMLCLK PTLEKKFFGK EFDDNIHIQL |
| 181 | IYNILDIEKI LAVYSTNAIY ALNNMSADEN IENSDFFMKR TTDETFDDFE KKKESTNSRE |
| 241 | KADFDAFEKF IGNYRLAYFA DAFYVNKKNP KGKAKNVLRE DKELYSVLTL IGKLAHWCVA |
| 301 | SEEGRAEFWL YKLDELKDDF KNVLDVVYNR PVEEINNRFI ENNKVNIQIL GSVYKNTDIA |
| 361 | ELVRSYYEFL ITKKYKNMGF SIKKLRESML EGKGYADKEY DSVRNKLYQM TDFILYTGYI |
| 421 | NEDSDRADDL VNTLRSSLKE DDKTTVYCKE ADYLWKKYRE AIREVADALD GDNIKKLSKS |
| 481 | NIEIQEDKLR KCFISYADSV SEFTKLIYLL TRFLSGKEIN DLVTTLINKF DNIRSFLEIM |
| 541 | DELGLDRTFT AEYSFFEGST KYLAELVELN SFVKSCSFDI NAKRTMYRDA LDILGIESDK |
| 601 | TEEDIEKMID NILQIDANGD KKLKKNNGLR NFIASNVIDS NRFKYLVRYG NPKKIRETAK |
| 661 | CKPAVRFVLN EIPDAQIERY YEACCPKNTA LCSANKRREK LADMIAEIKF ENFSDAGNYQ |
| 721 | KANVTSRTSE AEIKRKNQAI IRLYLTVMYI MLKNLVNVNA RYVIAFHCVE RDTKLYAESG |
| 781 | LEVGNIEKNK TNLTMAVMGV KLENGIIKTE FDKSFAENAA NRYLRNARWY KLILDNLKKS |
| 841 | ERAVVNEFAN TVCALNAIRN ININIKEIKE VENYFALYHY LIQKHLENRF ADKKVERDTG |
| 901 | DFISKLEEHK TYCKDFVKAY CTPFGYNLVR YKNLTIDGLF DKNYPGKDDS DEQK |

uncultured Ruminococcus sp. URCas13d (PDB: 6IV9_A)
(SEQ ID NO: 41)

| | |
|---|---|
| 1 | MAKKNKMKPR ELREAQKKAR QLKAAEINNN AAPAIAAMPA AEVIAPVAEK KKSSVKAAGM |
| 61 | KSILVSENKM YITSFGKGNS AVLEYEVDNN DYNKTQLSSK DNSNIELGDV NEVNITFSSK |
| 121 | KGFGSGVEIN TSNPTHRSGE SSPVRGDMLG LKSELEKRFF GKTFDDNIHI QLIYNILDIE |
| 181 | KILAVYVTNI VYALNNMLGI KDSESYDDFM GYLSARNTYE VFTHPDKSNL SDKVKGNIKK |
| 241 | SLSKFNDLLK TKRLGYFGLE EPKTKDTRAS EAYKKRVYHN LAIVGQIAQC VFHDKSGAKE |
| 301 | FDLYSFINNI DPEYRDTLDY LVEERLKSIN KDFIEGNKVN ISLLIDMMKG YEADDIIRLY |
| 361 | YDFIVLKSQK NLGFSIKKLR EKMLEEYGFR FKDKQYDSVR SKMYKLMDFL LFCNYYRNDV |
| 421 | AAGEALVRKL RFSMTDDEKE GIYADEAAKL WGKFRNDFEN IADHMNGDVI KELGKADMDF |
| 481 | DEKILDSEKK NASDLLYFSK MIYMLTYFLD GKEINDLLTT LISKFDNIKE FLKIMKSSAV |
| 541 | DVECELTAGY KLFNDSQRIT NELFIVKNIA SMRKPAASAK LTMFRDALTI LGIDDNITDD |
| 601 | RISEILKLKE KGKGIHGLRN FITNNVIESS RFVYLIKYAN AQKIREVAKN EKVVMFVLGG |
| 661 | IPDTQIERYY KSCVEFPDMN SSLEAKRSEL ARMIKNISFD DFKNVKQQAK GRENVAKERA |
| 721 | KAVIGLYLTV MYLLVKNLVN VNARYVIAIH CLERDFGLYK EIIPELASKN LKNDYRILSQ |
| 781 | TLCELCDDRN ESSNLFLKKN KRLRKCVEVD INNADSSMTR KYANCIAHLT VVRELKEYIG |
| 841 | DIRTVDSYFS IYHYVMQRCI TKRGDDTKQE EKIKYEDDLL KNHGYTKDFV KALNSPFGYN |
| 901 | IPRFKNLSIE QLFDRNEYLT EKLEHHHHHH |

1. An accessory nuclease activator of a Type III Cas protein, wherein activation of the Type III Cas protein as a non-specific nuclease is sustained at high levels and is not self-limited.

2. The activator of claim 1, wherein the Type III Cas protein is Csm6 or Csx1, optionally a T. thermophilus (TtCsm6) protein.

3. The activator of claim 1, comprising one or more cyclic and/or linear oligoadenylates.

4. The activator of claim 3, wherein the one or more cyclic and/or linear oligoadenylates comprise one or more modified bases and/or caging structures, optionally wherein the modification comprises substituting one or more bases with a non-naturally occurring base.

5. The activator of claim 1, wherein the activator comprises a linear A4 or A6 oligoadenylate.

6. The activator of claim 4, wherein the one or more modified bases comprise fluorinated, methylated and/or deoxy modified bases.

7. The activator of claim 5, wherein the activator comprises a substitution at position 2 (the second A) of the A4 oligoadenylate or position 3 (the third A) of the A6 oligoadenylate, optionally with a fluorine molecule to form A-fA-AA>P or AA-fA-AAA>P.

8. The activator of claim 1, comprising a molecule as shown in Table 3.

9. The activator of claim 1, further comprising additional sequences.

10. The activator of claim 1, wherein the activator comprises a sequence recognized by a different enzyme than the Type III Cas protein, optionally a Type VI Cas protein.

11. The activator of claim 10, wherein the sequence comprises a linear polyU chain of 1-10 U residues recognized by a Cas13 enzyme, optionally wherein the polyU sequence comprises one or more modified bases, optionally wherein the polyU sequence comprises 2′-deoxy modifications 3′ to the first U.

12. The activator of claim 1, further comprising a polyC sequence, optionally wherein the polyC sequence comprises one or more modified bases.

13. The activator of claim 1, further comprising one or more detectable labels, optionally a fluorescent label such as a fluorescein and/or one or more quenchers.

14. (canceled)

15. A nucleic acid detection system comprising one or more activators of claim 1 and the Type III Cas protein activated into a non-specific nuclease by the one or more Type III accessory nuclease activators, optionally further comprising one or more reporters that produces a detectable signal upon cleavage by the activated Type III Cas protein.

16. The nucleic acid detection system of claim 15, further comprising a Cas-based nucleic acid detection system comprising:
a Cas effector protein that is activated into a non-specific nuclease upon binding to a target sequence in a sample; and
at least one reporter that produces a detectable signal upon cleavage by the activated Cas effector protein.

17. The nucleic acid detection system of claim 16, the Cas effector protein is Cas13 protein, optionally a Cas13a protein, optionally a LbuCas13 protein.

18. The nucleic acid detection system of claim 15, wherein the activated Cas effector protein and activated Type III Cas protein cleave the same or different reporters.

19. A method of detecting one or more nucleic acid(s) in a sample, the method comprising:
contacting the sample with one or more nucleic acid detection systems according to claim 16, thereby detecting the nucleic acid in the sample, optionally, wherein the methods further comprise quantifying the levels of the detected signal.

20. A kit comprising one or more activators of claim 1.

21. A kit comprising one or more nucleic acid detection systems of claim 15.


SEQUENCE LISTING (44 sequences)

SEQ ID NO: 1 - RNA, 10 nt, artificial
  Type III accessory nuclease activator
aaaauuuuuu

SEQ ID NO: 2 - RNA, 10 nt, artificial
  Type III accessory nuclease activator
  modified_base (2)..(2): 2 (prime) fluoro modified A
  modified_base (8)..(8): 5-methylcytosine
  modified_base (9)..(9): 5-methylcytosine
aaaauccccc

SEQ ID NO: 3 - RNA, 60 nt, artificial
  Cas13 guide
auuuagaccaccccaaaaaugaaggggacuaaaacacagauauagccuggugguucaggc

SEQ ID NO: 4 - RNA, 28 nt, artificial
  Cas13 activator
aaaagccugaaccaccaggcuauaucug

SEQ ID NO: 5 - RNA, 60 nt, artificial
  Cas13 guide
uagaccagcccaaaaaugaagggcacuaaaacgcagcgccucuugcaacgauuaaauacu

SEQ ID NO: 6 - RNA, 30 nt, artificial
  Cas13 activator
aguauuuaaucguugcaagaggcgcugcuc

SEQ ID NO: 7 - RNA, 52 nt, artificial
  Cas13 guide
uagaccaccccaaaaaugaaggggacuaaaacguucggacaaagugcaugaa

SEQ ID NO: 8 - RNA, 32 nt, artificial
  Cas13 activator
uaaagcuucaugcacuuuguccgaacaacugg

SEQ ID NO: 9 - RNA, 52 nt, artificial
  Cas13 guide
uagaccaccccaaaaaugaaggggacuaaaacgguccaccaaacguaaugcg

SEQ ID NO: 10 - RNA, 52 nt, artificial
  Cas13 guide
uagaccaccccaaaaaugaaggggacuaaaacuuugcggccaauguuuguaa

SEQ ID NO: 11 - RNA, 2668 nt, artificial
  Type IV activator
cagguuacuauagcagagauauuacuaauuauuaugaggacuuuuaaaguuuccauuuggaaucuugauuacaucauaaaccucauaauuaaaaauuuaucuaagucacuaacugagaauaaauauucucaauuagaugaagagcaaccaauggagauugauuaaacgaacaugaaaauuauucuuuucuuggcacugauaacacucgcuacuugugagcuuuaucacuaccaagaguguguuagagguacaacaguacuuuuaaaagaaccuugcucuucuggaacauacgagggcaauucaccauuucauccucuagcugauaacaaauuugcacugacuugcuuuagcacucaauuugcuuuugcuuguccugacggcguaaaacacgucuaucaguuacgugccagaucaguuucaccuaaacuguucaucagacaagaggaaguucaagaacuuuacucuccaauuuuucuuauuguugcggcaauaguguuuauaacacuuugcuucacacucaaaagaaagacagaaugauugaacuuucauuaauugacuucuauuugugcuuuuuagccuuucugcuauuccuuguuuuaauuaugcuuauuaucuuuugguucucacuugaacugcaagaucauaaugaaacuugucacgccuaaacgaacaugaaauuucuuguuuucuuaggaaucaucacaacuguagcugcauuucaccaagaauguaguuuacagucauguacucaacaucaaccauauguaguugaugacccguguccuauucacuucuauucuaaaugguauauuagaguaggagcuagaaaaucagcaccuuuaauugaauugugcguggaugaggcugguucuaaaucacccauucaguacaucgauaucgguaauuauacaguuuccuguuuaccuuuuacaauuaauugccaggaaccuaaauuggguagucuuguagugcguuguucguucuaugaagacuuuuuagaguaucaugacguucguguuguuuuagauuucaucuaaacgaacaaacuaaaaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaa

SEQ ID NO: 12 - RNA, 10 nt, artificial
  Type III accessory nuclease activator
  modified_base (1)..(1): 2 (prime) OMe modified A
  modified_base (2)..(2): 2 (prime) OMe modified A
  modified_base (3)..(3): 2 (prime) OMe modified A
aaaauuuuuu

SEQ ID NO: 13 - RNA, 10 nt, artificial
  Type III accessory nuclease activator
  modified_base (1)..(1): 2 (prime) deoxy modified A
  modified_base (2)..(2): 2 (prime) deoxy modified A
  modified_base (3)..(3): 2 (prime) deoxy modified A
aaaauuuuuu

SEQ ID NO: 14 - RNA, 10 nt, artificial
  Type III accessory nuclease activator
  modified_base (2)..(2): 2 (prime) deoxy modified A
aaaauuuuuu

SEQ ID NO: 15 - RNA, 10 nt, artificial
  Type III accessory nuclease activator
  modified_base (1)..(1): 2 (prime) deoxy modified A
aaaauuuuuu

SEQ ID NO: 16 - RNA, 10 nt, artificial
  Type III accessory nuclease activator
  modified_base (3)..(3): 2 (prime) deoxy modified A
aaaauuuuuu

SEQ ID NO: 17 - RNA, 10 nt, artificial
  Type III accessory nuclease activator
  modified_base (2)..(2): 2 (prime) deoxy modified A
  modified_base (3)..(3): 2 (prime) deoxy modified A
aaaauuuuuu

SEQ ID NO: 18 - RNA, 10 nt, artificial
  Type III accessory nuclease activator
  modified_base (1)..(1): 2 (prime) deoxy modified A
  modified_base (2)..(2): 2 (prime) deoxy modified A
aaaauuuuuu

SEQ ID NO: 19 - RNA, 10 nt, artificial
  Type III accessory nuclease activator
  modified_base (1)..(1): 2 (prime) fluoro modified A
  modified_base (2)..(2): 2 (prime) fluoro modified A
  modified_base (3)..(3): 2 (prime) fluoro modified A
aaaauuuuuu

SEQ ID NO: 20 - RNA, 10 nt, artificial
  Type III accessory nuclease activator
  modified_base (2)..(2): 2 (prime) fluoro modified A
aaaauuuuuu

SEQ ID NO: 21 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 22 - protein, 1152 aa, Leptotrichia wadei
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENDITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 23 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 24 - protein, 1340 aa, artificial
  synthetic CasA polypeptide
MQISKVNHKHVAVGQKDRERITGFIYNDPVGDEKSLEDVVAKRANDTKVLFNVFNTKDLYDSQESDKSEKDKEIISKGAKFVAKSFNSAITILKKQNKIYSTLTSQQVIKELKDKFGGARIYDDDIEEALTETLKKSFRKENVRNSIKVLIENAAGIRSSLSKDEEELIQEYFVKQLVEEYTKTKLQKNVVKSIKNQNMVIQPDSDSQVLSLSESRREKQSSAVSSDTLVNCKEKDVLKAFLTDYAVLDEDERNSLLWKLRNLVNLYFYGSESIRDYSYTKEKSVWKEHDEQKANKTLFIDEICHITKIGKNGKEQKVLDYEENRSRCRKQNINYYRSALNYAKNNTSGIFENEDSNHFWIHLIENEVERLYNGIENGEEFKFETGYISEKVWKAVINHLSIKYIALGKAVYNYAMKELSSPGDIEPGKIDDSYINGITSFDYEIIKAEESLQRDISMNVVFATNYLACATVDTDKDFLLFSKEDIRSCTKKDGNLCKNIMQFWGGYSTWKNFCEEYLKDDKDALELLYSLKSMLYSMRNSSFHFSTENVDNGSWDTELIGKLFEEDCNRAARIEKEKFYNNNLHMFYSSSLLEKVLERLYSSHHERASQVPSFNRVFVRKNFPSSLSEQRITPKFTDSKDEQIWQSAVYYLCKEIYYNDFLQSKEAYKLFREGVKNLDKNDINNQKAADSFKQAVVYYGKAIGNATLSQVCQAIMTEYNRQNNDGLKKKSAYAEKQNSNKYKHYPLFLKQVLQSAFWEYLDENKEIYGFISAQIHKSNVEIKAEDFIANYSSQQYKKLVDKVKKTPELQKWYTLGRLINPRQANQFLGSIRNYVQFVKDIQRRAKENGNPIRNYYEVLESDSIIKILEMCTKLNGTTSNDIHDYFRDEDEYAEYISQFVNFGDVHSGAALNAFCNSESEGKKNGIYYDGINPIVNRNWVLCKLYGSPDLISKIISRVNENMIHDFHKQEDLIREYQIKGICSNKKEQQDLRTFQVLKNRVELRDIVEYSEIINELYGQLIKWCYLRERDLMYFQLGFHYLCLNNASSKEADYIKINVDDRNISGAILYQIAAMYINGLPVYYKKDDMYVALKSGKKASDELNSNEQTSKKINYFLKYGNNILGDKKDQLYLAGLELFENVAEHENIIIFRNEIDHFHYFYDRDRSMLDLYSEVFDRFFTYDMKLRKNVVNMLYNILLDHNIVSSFVFETGEKKVGRGDSEVIKPSAKIRLRANNGVSSDVFTYKVGSKDELKIATLPAKNEEFLLNVARLIYYPDMEAVSENMVREGVVKVEKSNDKKGKISRGSNTRSSNQSKYNNKSKNRMNYSMGSIFEKMDLKFD

SEQ ID NO: 25 - protein, 1437 aa, Lachnospiraceae bacterium
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 26 - protein, 1385 aa, Clostridium aminophilum
MKFSKVDHTRSAVGIQKATDSVHGMLYTDPKKQEVNDLDKRFDQLNVKAKRLYNVFNQSKAEEDDDEKRFGKVVKKLNRELKDLLFHREVSRYNSIGNAKYNYYGIKSNPEEIVSNLGMVESLKGERDPQKVISKLLLYYLRKGLKPGTDGLRMILEASCGLRKLSGDEKELKVFLQTLDEDFEKKTFKKNLIRSIENQNMAVQPSNEGDPIIGITQGRFNSQKNEEKSAIERMMSMYADLNEDHREDVLRKLRRLNVLYFNVDTEKTEEPTLPGEVDTNPVFEVWHDHEKGKENDRQFATFAKILTEDRETRKKEKLAVKEALNDLKSAIRDHNIMAYRCSIKVTEQDKDGLFFEDQRINRFWIHHIESAVERILASINPEKLYKLRIGYLGEKVWKDLLNYLSIKYIAVGKAVFHFAMEDLGKTGQDIELGKLSNSVSGGLTSFDYEQIRADETLQRQLSVEVAFAANNLFRAVVGQTGKKIEQSKSEENEEDFLLWKAEKIAESIKKEGEGNTLKSILQFFGGASSWDLNHFCAAYGNESSALGYETKFADDLRKAIYSLRNETFHFTTLNKGSFDWNAKLIGDMFSHEAATGIAVERTRFYSNNLPMFYRESDLKRIMDHLYNTYHPRASQVPSFNSVFVRKNFRLFLSNTLNTNTSFDTEVYQKWESGVYYLFKEIYYNSFLPSGDAHHLFFEGLRRIRKEADNLPIVGKEAKKRNAVQDFGRRCDELKNLSLSAICQMIMTEYNEQNNGNRKVKSTREDKRKPDIFQHYKMLLLRTLQEAFAIYIRREEFKFIFDLPKTLYVMKPVEEFLPNWKSGMFDSLVERVKQSPDLQRWYVLCKFLNGRLLNQLSGVIRSYIQFAGDIQRRAKANHNRLYMDNTQRVEYYSNVLEVVDFCIKGTSRFSNVFSDYFRDEDAYADYLDNYLQFKDEKIAEVSSFAALKTFCNEEEVKAGIYMDGENPVMQRNIVMAKLFGPDEVLKNVVPKVTREEIEEYYQLEKQIAPYRQNGYCKSEEDQKKLLRFQRIKNRVEFQTITEFSEIINELLGQLISWSFLRERDLLYFQLGFHYLCLHNDTEKPAEYKEISREDGTVIRNAILHQVAAMYVGGLPVYTLADKKLAAFEKGEADCKLSISKDTAGAGKKIKDFFRYSKYVLIKDRMLTDQNQKYTIYLAGLELFENTDEHDNITDVRKYVDHFKYYATSDENAMSILDLYSEIHDRFFTYDMKYQKNVANMLENILLRHFVLIRPEFFTGSKKVGEGKKITCKARAQIEIAENGMRSEDFTYKLSDGKKNISTCMIAARDQKYLNTVARLLYYPHEAKKSIVDTREKKNNKKTNRGDGTFNKQKGTARKEKDNGPREFNDTGFSNTPFAGFDPFRNS

SEQ ID NO: 27 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 28 - protein, 1164 aa, Carnobacterium gallinarum
MRMTKVKINGSPVSMNRSKLNGHLVWNGTTNTVNILTKKEQSFAASFLNKTLVKADQVKGYKVLAENIFIIFEQLEKSNSEKPSVYLNNIRRLKEAGLKRFFKSKYHEEIKYTSEKNQSVPTKLNLIPLFFNAVDRIQEDKFDEKNWSYFCKEMSPYLDYKKSYLNRKKEILANSIQQNRGFSMPTAEEPNLLSKRKQLFQQWAMKFQESPLIQQNNFAVEQFNKEFANKINELAAVYNVDELCTAITEKLMNFDKDKSNKTRNFEIKKLWKQHPHNKDKALIKLFNQEGNEALNQFNIELGKYFEHYFPKTGKKESAESYYLNPQTIIKTVGYQLRNAFVQYLLQVGKLHQYNKGVLDSQTLQEIGMYEGFQTKFMDACVFASSSLRNIIQATTNEDILTREKFKKELEKNVELKHDLFFKTEIVEERDENPAKKIAMTPNELDLWAIRGAVQRVRNQIFHQQINKRHEPNQLKVGSFENGDLGNVSYQKTIYQKLFDAEIKDIEIYFAEKIKSSGALEQYSMKDLEKLFSNKELTLSLGGQVVAFAPSYKKLYKQGYFYQNEKTIELEQFTDYDFSNDVFKANYYLIKLIYHYVFLPQFSQANNKLFKDTVHYVIQQNKELNTTEKDKKNNKKIRKYAFEQVKLMKNESPEKYMQYLQREMQEERTIKEAKKTNEEKPNYNFEKLLIQIFIKGFDTFLRNFDLNLNPAEELVGTVKEKAEGLRKRKERIAKILNVDEQIKTGDEEIAFWIFAKLLDARHLSELRNEMIKFKQSSVKKGLIKNGDLIEQMQPILELCILSNDSESMEKESFDKIEVFLEKVELAKNEPYMQEDKLTPVKFRFMKQLEKYQTRNFIENLVIENPEFKVSEKIVLNWHEEKEKIADLVDKRTKLHEEWASKAREIEEYNEKIKKNKSKKLDKPAEFAKFAEYKIICEAIENFNRLDHKVRLTYLKNLHYLMIDLMGRMVGFSVLFERDFVYMGRSYSALKKQSIYLNDYDTFANIRDWEVNENKHLFGTSSSDLTFQETAEFKNLKKPMENQLKALLGVTNHSFEIRNNIAHLHVLRNDGKGEGVSLLSCMNDLRKLMSYDRKLKNAVTKAIIKILDKHGMILKLTNNDHTKPFEIESLKPKKIIHLEKSNHSFPMDQVSQEYCDLVKKMLVFTN

SEQ ID NO: 29 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 30 - protein, 970 aa, Listeria weihenstephanensis
MLALLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKVVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGDFKKNLPNSTTQSDVVKKMIPFFSDEIQNVNFDEAIWAIRGSIQQIRNEVYHCKKHSWKSILKIKGFEFEPNNMKYTDSDMQKLMDKDIAKIPDFIEEKLKSSGIIRFYSHDKLQSIWEMKQGFSLLTTNAPFVPSFKRVYAKGHDYQTSKNRYYDLGLTTFDILEYGEEDFRARYFLTKLVYYQQFMPWFTADNNAFRDAANFVLRLNKNRQQDAKAFINIREVEEGEMPRDYMGYVQGQIAIHEDSTEDTPNHFEKFISQVFIKGFDSHMRSADLKFIKNPRNQGLEQSEIEEMSFDIKVEPSFLKNKDDYIAFWTFCKMLDARHLSELRNEMIKYDGHLTGEQEIIGLALLGVDSRENDWKQFFSSEREYEKIMKGYVGEELYQREPYRQSDGKTPILFRGVEQARKYGTETVIQRLFDASPEFKVSKCNITEWERQKETIEETIERRKELHNEWEKNPKKPQNNAFFKEYKECCDAIDAYNWHKNKTTLVYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGEKKIDNGYIETNQVSEEYCGIVKRLLEI

SEQ ID NO: 31 - protein, 1051 aa, Listeria newyorkensis
MKITKMRVDGRTIVMERTSKEGQLGYEGIDGNKTTEIIFDKKKESFYKSILNKTVRKPDEKEKNRRKQAINKAINKEITELMLAVLHQEVPSQKLHNLKSLNTESLTKLFKPKFQNMISYPPSKGAEHVQFCLTDIAVPAIRDLDEIKPDWGIFFEKLKPYTDWAESYIHYKQTTIQKSIEQNKIQSPDSPRKLVLQKYVTAFLNGEPLGLDLVAKKYKLADLAESFKLVDLNEDKSANYKIKACLQQHQRNILDELKEDPELNQYGIEVKKYIQRYFPIKRAPNRSKHARADFLKKELIESTVEQQFKNAVYHYVLEQGKMEAYELTDPKTKDLQDIRSGEAFSFKFINACAFASNNLKMILNPECEKDILGKGNFKKNLPNSTTRSDVVKKMIPFFSDELQNVNFDEAIWAIRGSIQQIRNEVYHCKKHSWKSILKIKGFEFEPNNMKYADSDMQKLMDKDIAKIPEFIEEKLKSSGVVRFYRHDELQSIWEMKQGFSLLTTNAPFVPSFKRVYAKGHDYQTSKNRYYNLDLTTFDILEYGEEDFRARYFLTKLVYYQQFMPWFTADNNAFRDAANFVLRLNKNRQQDAKAFINIREVEEGEMPRDYMGYVQGQIAIHEDSIEDTPNHFEKFISQVFIKGFDRHMRSANLKFIKNPRNQGLEQSEIEEMSFDIKVEPSFLKNKDDYIAFWIFCKMLDARHLSELRNEMIKYDGHLTGEQEIIGLALLGVDSRENDWKQFFSSEREYEKIMKGYVVEELYQREPYRQSDGKTPILFRGVEQARKYGTETVIQRLFDANPEFKVSKCNLAEWERQKETIEETIKRRKELHNEWAKNPKKPQNNAFFKEYKECCDAIDAYNWHKNKTTLAYVNELHHLLIEILGRYVGYVAIADRDFQCMANQYFKHSGITERVEYWGDNRLKSIKKLDTFLKKEGLFVSEKNARNHIAHLNYLSLKSECTLLYLSERLREIFKYDRKLKNAVSKSLIDILDRHGMSVVFANLKENKHRLVIKSLEPKKLRHLGGKKIDGGYIETNQVSEEYCGIVKRLLEM

SEQ ID NO: 32 - protein, 1152 aa, Leptotrichia wadei; identical to SEQ ID NO: 22

SEQ ID NO: 33 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRTSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 34 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 35 - protein, 1285 aa, Rhodobacter capsulatus; identical to SEQ ID NO: 34

SEQ ID NO: 36 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 37 - protein, 1285 aa, Rhodobacter capsulatus; identical to SEQ ID NO: 34

SEQ ID NO: 38 - protein, 1344 aa, Eubacterium rectale
MLRRDKEVKKLYNVFNQIQVGTKPKKWNNDEKLSPEENERRAQQKNIKMKNYKWREACSKYVESSQRIINDVIFYSYRKAKNKLRYMRKNEDILKKMQEAEKLSKFSGGKLEDFVAYTLRKSLVVSKYDTQEFDSLAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKDAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPSYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNARSLNLMVGSMRSYIQYVTDIKRRAASIGNELHVSVHDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANLNFKLSN

SEQ ID NO: 39 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 40 - protein, 954 aa, Eubacterium siraeum
MGKKIHARDLREQRKTDRTEKFADQNKKREAERAVPKKDAAVSVKSVSSVSSKKDNVTKSMAKAAGVKSVFAVGNTVYMTSFGRGNDAVLEQKIVDTSHEPLNIDDPAYQLNVVTMNGYSVTGHRGETVSAVTDNPLRRFNGRKKDEPEQSVPTDMLCLKPTLEKKFFGKEFDDNIHIQLIYNILDIEKILAVYSTNAIYALNNMSADENIENSDFFMKRTTDETFDDFEKKKESTNSREKADFDAFEKFIGNYRLAYFADAFYVNKKNPKGKAKNVLREDKELYSVLTLIGKLAHWCVASEEGRAEFWLYKLDELKDDFKNVLDVVYNRPVEEINNRFIENNKVNIQILGSVYKNTDIAELVRSYYEFLITKKYKNMGFSIKKLRESMLEGKGYADKEYDSVRNKLYQMTDFILYTGYINEDSDRADDLVNTLRSSLKEDDKTTVYCKEADYLWKKYRESIREVADALDGDNIKKLSKSNIEIQEDKLRKCFISYADSVSEFTKLIYLLTRFLSGKEINDLVTTLINKFDNIRSFLEIMDELGLDRTFTAEYSFFEGSTKYLAELVELNSFVKSCSFDINAKRTMYRDALDILGIESDKTEEDIEKMIDNILQIDANGDKKLKKNNGLRNFIASNVIDSNRFKYLVRYGNPKKIRETAKCKPAVRFVLNEIPDAQIERYYEACCPKNTALCSANKRREKLADMIAEIKFENFSDAGNYQKANVTSRTSEAEIKRKNQAIIRLYLTVMYIMLKNLVNVNARYVIAFHCVERDTKLYAESGLEVGNIEKNKTNLTMAVMGVKLENGIIKTEFDKSFAENAANRYLRNARWYKLILDNLKKSERAVVNEFANTVCALNAIRNININIKEIKEVENYFALYHYLIQKHLENRFADKKVERDTGDFISKLEEHKTYCKDFVKAYCTPFGYNLVRYKNLTIDGLFDKNYPGKDDSDEQK

SEQ ID NO: 41 - protein, 930 aa, unknown
  uncultured Ruminococcus sp.
MAKKNKMKPRELREAQKKARQLKAAEINNNAAPAIAAMPAAEVIAPVAEKKKSSVKAAGMKSILVSENKMYITSFGKGNSAVLEYEVDNNDYNKTQLSSKDNSNIELGDVNEVNITFSSKHGFGSGVEINTSNPTHRSGESSPVRGDMLGLKSELEKRFFGKTFDDNIHIQLIYNILDIEKILAVYVTNIVYALNNMLGIKDSESYDDFMGYLSARNTYEVFTHPDKSNLSDKVKGNIKKSLSKFNDLLKTKRLGYFGLEEPKTKDTRASEAYKKRVYHMLAIVGQIAQCVFHDKSGAKRFDLYSFINNIDPEYRDTLDYLVEERLKSINKDFIEGNKVNISLLIDMMKGYEADDIIRLYYDFIVLKSQKNLGFSIKKLREKMLEEYGFRFKDKQYDSVRSKMYKLMDFLLFCNYYRNDVAAGEALVRKLRFSMTDDEKEGIYADEAAKLWGKFRNDFENIADHMNGDVIKELGKADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDNITDDRISEILKLKEKGKGIHGLRNFITNNVIESSRFVYLIKYANAQKIREVAKNEKVVMFVLGGIPDTQIERYYKSCVEFPDMNSSLEAKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDDRNESSNLFLKKNKRLRKCVEVDINNADSSMTRKYANCIAHLTVVRELKEYIGDIRTVDSYFSIYHYVMQRCITKRGDDTKQEEKIKYEDDLLKNHGYTKDFVKALNSPFGYNIPRFKNLSIEQLFDRNEYLTEKLEHHHHHH

SEQ ID NO: 42 - RNA, 10 nt, artificial
  Type III accessory nuclease activator
  modified_base (2)..(2): 2 (prime) fluoro modified A
aaaucccccc

SEQ ID NO: 43 - RNA, 10 nt, artificial
  Type III accessory nuclease activator
  modified_base (2)..(2): 2 (prime) fluoro modified A
  modified_base (5)..(5): 2 (prime) deoxycytidine modified C
  modified_base (6)..(6): 2 (prime) deoxycytidine modified C
  modified_base (7)..(7): 2 (prime) deoxycytidine modified C
  modified_base (8)..(8): 2 (prime) deoxycytidine modified C
  modified_base (9)..(9): 2 (prime) deoxycytidine modified C
  modified_base (10)..(10): 2 (prime) deoxycytidine modified C
aaaacccccc

SEQ ID NO: 44 - protein, 1437 aa, Lachnospiraceae bacterium; identical to SEQ ID NO: 25